(19) United States
(12) Patent Application Publication
(10) Pub. No.: US 2009/0275636 A1
(43) Pub. Date: Nov. 5, 2009

(54) PICORNAVIRUS AND USES THEREOF

(75) Inventors: Thomas Briese, White Plains, NY (US); Gustavo Palacios, New York, NY (US); W. Ian Lipkin, New York, NY (US); Daryl Lamson, Slingerlands, NY (US)

Correspondence Address: WilmerHale/Columbia University, 399 PARK AVENUE, NEW YORK, NY, 10022, US

(73) Assignee: The Trustees of Columbia University in the City of New York, New York, NY (US)

(21) Appl. No.: 12/364,301

(22) Filed: Feb. 2, 2009

Related U.S. Application Data
(63) Continuation-in-part of application No. PCT/US2007/017088, filed on Jul. 31, 2007.
(60) Provisional application No. 60/834,392, filed on Jul. 31, 2006.

(51) Int. Cl. A61K 31/7105; C12N 15/11; C07K 14/00; C07K 16/00; C12Q 1/70

(52) U.S. Cl. 514/44 A; 536/23.1; 530/350; 530/387.9; 530/387.3; 435/5

(57) ABSTRACT

The invention is directed to a a clade of newly isolated and identified picornaviruses associated with respiratory infection, and isolated nucleic acids sequences and peptides thereof. The invention also relates to antibodies against antigens derived from the picornavirus. The invention also relates to iRNAs which target nucleic acid sequences of the picornavirus. The invention is related to methods for detecting the presence or absence of picornavirus in a subject. The invention is also related to immunogenic compositions for inducing an immune response against picornavirus in a subject.

This application is a continuation-in-part of International Application No. PCT/US2007/017088, filed on Jul. 31, 2007, and claiming priority to U.S. Provisional Application No. 60/834,392, filed on Jul. 31, 2006.

The invention was made with government support by National Institutes of Health awards UC1 AI062705, U54 AI05715803, AI51292, AI056118, AI55466, U54AI57158 (Northeast Biodefense Center), New York State Department of Health Commissioner's Priority Pool Fund, and Cooperative Research Agreement U50/CCU223671 from the Centers for Disease Control and Prevention (CDC).

The content of all patent applications, published patents applications, issued and granted patents, and all references cited in this application are hereby incorporated by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described herein.

### BACKGROUND

Influenza-like illness (ILI), a non-specific respiratory illness defined as fever greater than 38° C. with cough and/or pharyngitis, is tracked by the Centers for Disease Control and Prevention (CDC) Influenza Surveillance System. Laboratory diagnosis of ILI is typically performed by virus isolation in cell culture, antigen detection, and nucleic acid amplification methods. Although multiplex nucleic acid amplification systems for detection of multiple respiratory pathogens have been described (Fan et al., 1998; Coiras et al, 2004; Syrmis et al., 2004; Templeton et al., 2004; Khanna et al., 2005) they are not yet widely implemented, primarily for reasons of complexity and cost.

ILI is a significant cause of morbidity and mortality in the US, accounting annually for approximately 36,000 deaths, 150,000 hospitalizations, and up to $12 billion in direct and indirect costs (Schoub and Martin, 2006). The advent of sensitive, affordable methods for differential diagnosis of the infectious agents that can cause ILI has the potential to reduce the economic burden afforded by these agents, to influence vaccine development and to improve clinical outcomes by facilitating early selection of appropriate antimicrobials. The importance of developing sound strategies for triaging patients with acute respiratory infection to specific treatment regimens is underscored in the context of pandemic influenza preparedness and the limited supply of influenza antiviral drugs.

### SUMMARY OF THE INVENTION

The invention is related to a novel picornavirus associated with influenza like illness, and isolated nucleic acids sequences and peptides thereof. The invention is also related to antibodies against antigens derived from the novel picornavirus. The invention is also related to iRNAs which target nucleic acid sequences of the novel picornavirus. The invention is related to methods for detecting the presence or absence of picornavirus in a subject. The invention is also related to immunogenic compositions for inducing an immune response against picornavirus in a subject.

In one aspect, the invention provides an isolated nucleic acid which comprises consecutive nucleotides having a sequence selected from the group consisting of any of: SEQ ID NO: 1 through SEQ ID NO: 23, and a variant of any one of SEQ ID NOS 1-23 having at least about 85% identity to SEQ ID NO: 1-23. In one embodiment of the above aspect of the invention, the variant has at least about 90%, about 95.5%, about 96%, about 96.5%, about 97%, about 97.5%, about 98%, about 98.5%, about 99%, about 99.5% or about 99.9% identity to that of any one of SEQ ID NO: 1-23. In another embodiment of the above aspect of the invention, the nucleic acid comprises consecutive nucleotides having a sequence substantially identical to any one of SEQ ID NO: 1-23.

In another aspect, the invention provides an isolated nucleic acid which comprises consecutive nucleotides having a sequence complementary to the nucleic acid of any of SEQ ID NO: 1-23. In a further aspect, the invention provides for an isolated nucleic acid consisting essentially of consecutive nucleotides having a sequence selected from the group consisting of: SEQ ID NO: 1-23, and a variant of any one of SEQ ID NOS 1-23 having at least about 95% identity to SEQ ID NO: 1-23. In one embodiment of the above aspect of the invention, the variant has at least about 95.5%, about 96%, about 96.5%, about 97%, about 97.5%, about 98%, about 98.5%, about 99%, about 99.5% or about 99.9% identity to SEQ ID NO: 1-23.

In a further aspect, the invention provides an isolated nucleic acid consisting of consecutive nucleotides having a sequence selected from the group consisting of: SEQ ID NO: 1-23, and a variant of any one of SEQ ID NOS 1-23 having at least about 95% identity to SEQ ID NO: 1-23. In one embodiment of the above aspect of the invention, the variant has at least about 95.5%, about 96%, about 96.5%, about 97%, about 97.5%, about 98%, about 98.5%, about 99%, about 99.5% or about 99.9% identity to SEQ ID NO: 1-23.

Also provided by the invention is an isolated nucleic acid that hybridizes to an isolated nucleic acid which comprises consecutive nucleotides having a sequence selected from the group consisting of: SEQ ID NO:1 through SEQ ID NO: 23, and a variant of any one of SEQ ID NOS 1-23 having at least about 95% identity to SEQ ID NO: 1-23 under conditions of high stringency.

In another aspect, the invention provides for an isolated nucleic acid which comprises consecutive nucleotides having a sequence selected from the group consisting of: SEQ ID NO: 1 through SEQ ID NO: 23, and a variant of any one of SEQ ID NOS 1-23 having at least about 95% identity to SEQ ID NO: 1-23 under conditions of moderate stringency.

Also provided by the invention is an isolated nucleic acid that hybridizes to an isolated nucleic acid which comprises consecutive nucleotides having a sequence selected from the group consisting of: SEQ ID NO:1 through SEQ ID NO: 23, and a variant of any one of SEQ ID NO: 1-23 having at least about 95% identity to SEQ ID NO: 1-23 under conditions of low stringency.

In one embodiment, the invention provides for an isolated nucleic acid which comprises consecutive nucleotides having a sequence selected from the group consisting of: SEQ ID NO:1 through SEQ ID NO: 23, and a variant of any one of SEQ ID NO: 1-23 having at least about 95% identity to SEQ ID NO: 1-23, wherein the variant has at least about 95% identity to any one of SEQ ID NO: 1-23, as determined by analysis with a sequence comparison algorithm. In another embodiment, the sequence comparison algorithm is FASTA version 3.0t78 using default parameters.

The invention also provides for an isolated nucleic acid comprising at least fifteen (15) consecutive nucleotides having a sequence identical to a portion of any sequence selected from the group consisting of: SEQ ID NO: 1-23, a sequence substantially identical to any one of SEQ ID NO: 1-23, a sequence complementary to any one of SEQ ID NO:1-23. In another aspect, the invention provides for an isolated nucleic acid having at least about 95% identity to the nucleic acid of any one of SEQ ID NO: 1-23, a sequence substantially identical to and one of SEQ ID NO: 1-23 or a sequence complementary to any one of SEQ ID NO: 1-23 as determined by analysis with a sequence comparison algorithm or FASTA version 3.0t78 using default parameters as determined by analysis with a sequence comparison algorithm or FASTA version 3.0t78 using default parameters.

The invention also provides for an isolated nucleic acid encoding a polypeptide comprising consecutive amino acids having a sequence selected from the group consisting of: SEQ ID NO: 24-35, and an amino acid sequence substantially identical to any one of SEQ ID NO: 24-35. In one embodiment, the invention provides for a polypeptide having a sequence selected from the group consisting of: SEQ ID NO: 24-35. In other embodiments, the invention provides for an isolated polypeptide having at least about 95% identity, at least about 95.5%, about 96%, about 96.5%, about 97%, about 97.5%, about 98%, about 98.5%, about 99%, about 99.5% or about 99.9% identity to SEQ ID NO. 24-35.

In another aspect, the invention provides for an isolated antibody that binds to encoding a polypeptide comprising consecutive amino acids having a sequence selected from the group consisting of: SEQ ID NO: 24-35, and an amino acid sequence substantially identical to any one of SEQ ID NO: 24-35. In one embodiment, the invention provides for an antibody that binds a polypeptide having a sequence selected from the group consisting of: SEQ ID NO: 24-35. In other embodiments, the invention provides for an isolated polypeptide having at least about 95% identity, at least about 95.5%, about 96%, about 96.5%, about 97%, about 97.5%, about 98%, about 98.5%, about 99%, about 99.5% or about 99.9% identity to SEQ ID NO. 24-35. In other embodiments, the antibody is a polyclonal antibody, a monoclonal antibody, a human or humanized antibody or a chimeric antibody.

In yet another aspect, the invention provides a method for producing an isolated nucleic acid encoding a polypeptide comprising consecutive amino acids having a sequence selected from the group consisting of: SEQ ID NO: 24-35, and an amino acid sequence substantially identical to any one of SEQ ID NO: 24-35, the method comprising: (a) introducing a nucleic acid encoding the polypeptide into a host cell under conditions that permit expression of the polypeptide by the host cell, and (b) recovering the polypeptide.

In yet a further aspect, the invention provides a computer readable medium having stored thereon (i) a nucleic acid sequence selected from the group consisting of: SEQ ID NO: 1-23, and a sequence substantially identical to any one of the nucleic acid sequences; or (ii) an amino acid sequence selected from the group consisting of: SEQ ID NO: 1-23, and a sequence substantially identical to any one of the amino acid sequences.

In another aspect, the invention provides a method for comparing a first sequence to a second sequence, which comprises: (a) inputting the first sequence and the second sequence into a computer; (b) running a sequence comparison program on the computer so as to compare the first sequence with the second sequence; and (c) identifying differences between the first sequence and the second sequence thereby comparing the first sequence with the second sequence, wherein the first sequence comprises a sequence from any one of the sequences selected from the group consisting of SEQ ID NO: 1-23, or a sequence substantially identical to any one of the sequences, or any combination thereof.

In another aspect, the invention provides an oligonucleotide probe which comprises from about 10 nucleotides to about 50 nucleotides, wherein at least about 10 contiguous nucleotides are at least 95% complementary to a nucleic acid target region within a nucleic acid sequence selected from the group consisting of: SEQ ID NO: 1-23 wherein the oligonucleotide probe hybridizes to the nucleic acid target region under moderate to highly stringent conditions to form a detectable nucleic acid target:oligonucleotide probe duplex. In one embodiment, the oligonucleotide probe is at least about 95.5%, about 96%, about 96.5%, about 97%, about 97.5%, about 98%, about 98.5%, about 99%, about 99.5% or about 99.9% complementary to SEQ ID NO. 1-23. In another embodiment the oligonucleotide probe consists essentially of from about 10 to about 50 nucleotides.

Another aspect of the invention is a replicable nucleic acid vector which comprises an isolated nucleic acid consisting of consecutive nucleotides having a sequence selected from the group consisting of: SEQ ID NO: 1-23, and a variant of any one of SEQ ID NOS 1-23 having at least about 95% identity to SEQ ID NO: 1-23. In one embodiment of the above aspect of the invention, the variant has at least about 95.5%, about 96%, about 96.5%, about 97%, about 97.5%, about 98%, about 98.5%, about 99%, about 99.5% or about 99.9% identity to SEQ ID NO: 1-23. In one embodiment, the replicable nucleic acid vector is a viral vectors. Such vectors may include but are not limited to adenovirus, adeno-associated virus, lentivirus, and vesiculostomatitis virus vectors.

In yet another aspect, the invention provides for a host organism comprising a replicable nucleic acid vector which comprises an isolated nucleic acid consisting of consecutive nucleotides having a sequence selected from the group consisting of: SEQ ID NO: 1-23, and a variant of any one of SEQ ID NOS 1-23 having at least about 95% identity to SEQ ID NO: 1-23. In one embodiment of the above aspect of the invention, the variant has at least about 95.5%, about 96%, about 96.5%, about 97%, about 97.5%, about 98%, about 98.5%, about 99%, about 99.5% or about 99.9% identity to SEQ ID NO. In one embodiment, the replicable nucleic acid vector is a viral vectors. In another embodiment, the replicable nucleic acid vector is an adenovirus vector, a retroviral vector, or an adeno-associated viral (AAV) vector. In one embodiment, the host organism is a prokaryote, a eukaryote, or a fungus.

In another aspect, the invention provides for an immunogenic composition comprising at least a portion of a polypeptide comprising consecutive amino acids having a sequence selected from the group consisting of: SEQ ID NO: 24-35, and an amino acid sequence substantially identical to any one of SEQ ID NO: 24-35 or an isolated nucleic acid consisting of consecutive nucleotides having a sequence selected from the group consisting of: SEQ ID NO: 1-23, and a variant of any one of SEQ ID NOS 1-23 having at least about 95% identity to SEQ ID NO: 1-23.

In another aspect, the invention provides a method of inducing an immune response in a subject, the method comprising administering the immunogenic composition of at least a portion of a polypeptide comprising consecutive amino acids having a sequence selected from the group consisting of: SEQ ID NO: 24-35, and an amino acid sequence substantially identical to any one of SEQ ID NO: 24-35 or an isolated nucleic acid consisting of consecutive nucleotides having a sequence selected from the group consisting of: SEQ ID NO: 1-23, and a variant of any one of SEQ ID NOS 1-23 having at least about 95% identity to SEQ ID NO: 1-23

In yet a further aspect, the invention provides a synthetic nucleic acid which has a sequence consisting of from about 10 to about 30 consecutive nucleotides from a nucleic acids sequence selected from the group of sequences consisting of SEQ ID NO: 1-23.

In another aspect, the invention provides a composition comprising one or more nucleic acids having a sequence consisting of from about 10 to about 30 consecutive nucleotides from a nucleic acids sequence selected from the group of sequences consisting of SEQ ID NO: 1-23.

In another aspect, the invention provides a method for determining the presence or absence of the novel picornavirus in a biological sample, the method comprising: a) contacting nucleic acid from a biological sample with at least one primer which is a synthetic nucleic acid which has a sequence consisting of from about 10 to about 30 consecutive nucleotides from a nucleic acids sequence selected from the group of sequences consisting of SEQ ID NO: 1-23, b) subjecting the nucleic acid and the primer to amplification conditions, and c) determining the presence or absence of amplification product, wherein the presence of amplification product indicates the presence of RNA associated with picornavirus in the sample.

In yet another aspect, the invention provides a synthetic nucleic acid which has a sequence consisting of from about 10 to about 30 consecutive nucleotides from a nucleic acids sequence which is complementary to a nucleic acid sequence selected from the group of sequences consisting of SEQ ID NO: 1-23.

In yet another aspect, the invention provides a composition comprising one or more synthetic nucleic acids which have a sequence consisting of from about 10 to about 30 consecutive nucleotides from a nucleic acids sequence which is complementary to a nucleic acid sequence selected from the group of sequences consisting of SEQ ID NO: 1-23.

In an further aspect, the invention provides a method for determining the presence or absence of a novel picornavirus in a biological sample, the method comprising: a) contacting nucleic acid from a biological sample with at least one primer which is a nucleic acid sequence consisting of from about 10 to about 30 consecutive nucleotides from a nucleic acids sequence which is complementary to a nucleic acid sequence selected from the group of sequences consisting of SEQ ID NO: 1-23, b) subjecting the nucleic acid and the primer to amplification conditions, and c) determining the presence or absence of amplification product, wherein the presence of amplification product indicates the presence of RNA associated with picornavirus in the sample. In one embodiment, the biological sample is derived from a subject suspected of having a novel picornavirus.

In a further aspect, the invention provides a primer set for determining the presence or absence of the novel picornavirus in a biological sample, wherein the primer set comprises at least one synthetic nucleic acid sequence selected from the group consisting of: a synthetic nucleic acid which has a sequence consisting of from about 10 to about 30 consecutive nucleotides from a nucleic acids sequence selected from the group of sequences consisting of SEQ ID NO: 1-23, a synthetic nucleic acid which has a sequence consisting of from about 10 to about 30 consecutive nucleotides from a nucleic acids sequence which is complementary to a nucleic acid sequence selected from the group of sequences consisting of SEQ ID NO: 1-23. In one embodiment, the biological sample is derived from a subject suspected of having the novel picornavirus.

In another aspect, the invention provides a method for determining whether or not a sample contains picornavirus HRV-NY, the method comprising: (a) providing an immunoassay comprising an antibody against a picornavirus HRV-NY derived antigen, (b) contacting the antibody with a biological sample, (c) detecting binding between antigens in the test sample and the antibody. In one embodiment, the immunoassay is a lateral flow assay or ELISA. In one embodiment, the biological sample is derived from a subject suspected of having a picornavirus HRV-NY.

In still a further aspect, the invention provides a method for determining whether or not a sample contains antibodies against picornavirus HRV-NY, the method comprising: (a) providing an immunoassay comprising an antigen from a picornavirus HRV-NY, (b) contacting the antigen with a biological sample, (c) detecting binding between antibodies in the test sample and the antigen.

In yet another aspect, the invention provides a method for preparing a pharmaceutical composition which comprises admixing a pro-drug with the polypeptide or fragment thereof of a polypeptide encoded from an isolated nucleic acid having at least about 95% identity to the nucleic acid of any one of SEQ ID NO: 1-23, a sequence substantially identical to and one of SEQ ID NO: 1-23 or a sequence complementary to any one of SEQ ID NO: 1-23 as determined by analysis with a sequence comparison algorithm or FASTA version 3.0t78 using default parameters, a polypeptide encoded from an isolated nucleic acid encoding a polypeptide comprising consecutive amino acids having a sequence selected from the group consisting of: SEQ ID NO: 24-35, and an amino acid sequence substantially identical to any one of SEQ ID NO: 24-35, an isolated polypeptide having at least about 95% identity to the polypeptide of an isolated polypeptide encoded by any one of SEQ ID NO: 1-23, an isolated polypeptide encoded by a sequence substantially identical to and one of SEQ ID NO: 1-23 or an isolated polypeptide encoded by a sequence complementary to any one of SEQ ID NO: 1-23 as determined by analysis with a sequence comparison algorithm or FASTA version 3.0t78 using default parameters, an isolated polypeptide encoded by an isolated nucleic acid having at least about 95% identity to the nucleic acid of any one of SEQ ID NO: 1-23, an isolated polypeptide encoded by a sequence substantially identical to and one of SEQ ID NO: 1-23 or an isolated polypeptide encoded by a sequence complementary to any one of SEQ ID NO: 1-23 as determined by analysis with a sequence comparison algorithm or FASTA version 3.0t78 using default parameters or an isolated nucleic acid encoding a polypeptide comprising consecutive amino acids having a sequence selected from the group consisting of any of SEQ ID NO: 24-35 and an amino acid sequence substantially identical to any one of SEQ ID NO: 24-35, or an isolated polypeptide that has at least about 95.5%, about 96%, about 96.5%, about 97%, about 97.5%, about 98%, about 98.5%, about 99%, about 99.5% or about 99.9% identity to SEQ ID NO. 24-35, thereby preparing the pharmaceutical composition.

In yet another embodiment, the invention provides a method for treating or preventing a disease or condition in a subject, the method comprising administering to the subject a polypeptide or fragment thereof of a polypeptide encoded from an isolated nucleic acid having at least about 95% identity to the nucleic acid of any one of SEQ ID NO: 1-23, a sequence substantially identical to and one of SEQ ID NO: 1-23 or a sequence complementary to any one of SEQ ID NO: 1-23 as determined by analysis with a sequence comparison algorithm or FASTA version 3.0t78 using default parameters as determined by analysis with a sequence comparison algorithm or FASTA version 3.0t78 using default parameters, a polypeptide encoded from an isolated nucleic acid encoding a polypeptide comprising consecutive amino acids having a sequence selected from the group consisting of: SEQ ID NO: 24-35, and an amino acid sequence substantially identical to any one of SEQ ID NO: 24-35, an isolated polypeptide having at least about 95% identity to the polypeptide of an isolated polypeptide encoded by any one of SEQ ID NO: 1-23, an isolated polypeptide encoded by a sequence substantially identical to and one of SEQ ID NO: 1-23 or an isolated polypeptide encoded by a sequence complementary to any one of SEQ ID NO: 1-23 as determined by analysis with a sequence comparison algorithm or FASTA version 3.0t78 using default parameters, an isolated polypeptide encoded by an isolated nucleic acid having at least about 95% identity to the nucleic acid of any one of SEQ ID NO: 1-23, an isolated polypeptide encoded by a sequence substantially identical to and one of SEQ ID NO: 1-23 or an isolated polypeptide encoded by a sequence complementary to any one of SEQ ID NO: 1-23 as determined by analysis with a sequence comparison algorithm or FASTA version 3.0t78 using default parameters or an isolated nucleic acid encoding a polypeptide comprising consecutive amino acids having a sequence selected from the group consisting of any of SEQ ID NO: 24-35 and an amino acid sequence substantially identical to any one of SEQ ID NO: 24-35, or an isolated polypeptide that has at least about 95.5%, about 96%, about 96.5%, about 97%, about 97.5%, about 98%, about 98.5%, about 99%, about 99.5% or about 99.9% identity to SEQ ID NO. 24-35, so as to treat or prevent a disease or condition in the subject.

In still a further aspect, the invention provides for an interfering RNA (iRNA) comprising a sense strand having at least 15 contiguous nucleotides complementary to the anti-sense strand of a gene from a virus. In one embodiment, the gene is selected from the group consisting of VP1, VP2, VP3, VP4, Protein 2A, Protein 2B, Protein 2C, Protein 3A, Protein 3B, Protein 3C of picornavirus.

In yet another embodiment, the invention provides for an interfering RNA (iRNA) comprising an anti-sense strand having at least 15 contiguous nucleotides complementary to the sense strand of gene from a virus. In one embodiment, the gene is selected from the group consisting of VP1, VP2, VP3, VP4, Protein 2A, Protein 2B, Protein 2C, Protein 3A, Protein 3B, Protein 3C of picornavirus.

In still another aspect, the invention provides a method of reducing the levels of a viral protein, viral mRNA or viral titer in a cell in a subject comprising: administering at least one iRNA agent to a subject, wherein the iRNA agent comprising a sense strand having at least 15 contiguous nucleotides complementary to gene from a picornavirus comprising any of SEQ ID NO: 1-23 and an antisense strand having at least 15 contiguous nucleotides complementary to the sense strand. In one embodiment, the iRNA agent is administered intranasally to a subject. In another embodiment, the iRNA agent is administered via inhalation or nebulization to a subject. In yet another embodiment, the method further comprises co-administering a second iRNA agent to the subject, wherein the second iRNA agent comprising a sense strand having at least 15 or more contiguous nucleotides complementary to second gene from the picornavirus, and an antisense strand having at least 15 or more contiguous nucleotides complementary to the sense strand. In yet another embodiment, the subject is diagnosed as having a viral infection with the first and the second mammalian respiratory virus. In a further embodiment, the iRNA agent reduces the level of VP1 or VP4.

In another aspect, the invention provides a method of reducing the levels of a viral protein in a cell in a subject comprising the step of administering an iRNA agent to a subject, wherein the iRNA agent comprises a sense strand having at least 15 or more contiguous nucleotides complementary to a gene from a picornavirus comprising SEQ ID NO: 1-23 and an antisense strand having at least 15 or more contiguous nucleotides complementary to the sense strand. In one embodiment, the iRNA agent reduces the level of VP1 or VP4.

In yet another aspect, the invention provides an isolated virus comprising any one of SEQ ID NO: 1-23.

### BRIEF DESCRIPTION OF THE FIGURES

FIG. 1 shows a dendogram of isolates of the novel picornavirus and selected enterovirus and rhinovirus reference isolates. VP4 nucleotide sequence was used to reconstruct a phylogenetic tree with the Neighbor-Joining method applying a Kimura two-parameter model. Scale bar indicates nucleotide substitutions per site; bootstrap values (percentage of 1000 pseudo-replicates) are given at relevant branches. New York isolates related to HRV group A are indicated by black circle (), isolates related to HRV group B by black diamond (♦). Black triangle (▴) indicates isolates that cluster as a distinct genetic clade, HRV NY.

FIGS. 2A-2D show the nucleic acid sequence of SEQ ID: NO 1 which is derived from an HRV-NY virus.

FIGS. 3A-3B show nucleic acid sequences of SEQ ID NO: 2 which is derived from an HRV-NY virus.

FIGS. 4-11 show nucleic acid sequences of SEQ ID NOS: 3-10 which encode VP4 proteins of the novel picornavirus.

FIGS. 12-22 show nucleic acid sequence of SEQ ID NOS: 11-21 which are derived from 5′-UTR regions of the novel picornavirus.

FIGS. 23A and B shows the amino acid sequence of Isolate 1078 (SEQ ID NO: 24)

FIG. 24 shows the consensus sequence UTR, VP4, VP2, VP3, VP1 amino acid sequence of isolate 1064 (SEQ ID NO:25)

FIG. 25 shows a phylogenetic analysis of VP4/2 coding region of viruses identified in association with pediatric respiratory disease in Germany. Neighbor-Joining analysis of VP4/2 nucleotide sequence was performed by applying a Kimura 2-parameter model. Sequences belonging to the novel genotype recently identified in New York State (NY-003, -028, -42, -60, and -078); selected HRV-A serotypes (GenBank Accession numbers for all reference sequences are indicated in parenthesis); HRV-B serotypes; HEV-C viruses Human coxsackievirus A1, A21, and A24 (CV-A1, CV-A21, and CV-A24, respectively); Human poliovirus 2 (HPV-2); HEV-B viruses Human echovirus 5 and 6 (EV-5, EV-6), Human coxsackievirus B4 (CV-B4), and Swine vesicular disease virus (CV-B5); HEV-D viruses Human enterovirus 68 and 70 (HEV-68, HEV-70); Porcine enterovirus B virus Porcine enterovirus 9 (PEV-9); Bovine enterovirus virus Bovine enterovirus 1 (BEV-1); and HEV-A viruses Human coxsackievirus A16 (CV-A16), and Human enterovirus 71 (HEV-71) were included for comparison. Major clinical symptoms connected to the specimen in that the respective virus was detected are indicated in square brackets: cough [C], fever [F], rhinitis [R], pharyngitis [Ph], bronchitis/bronchiolitis [B], pneumonia [P].

FIG. 26 shows the amino acid sequence of isolate 1078.

FIGS. 27-35 show amino acid sequence of SEQ ID NOS: 27-35, which represent HRV-NY VP4 proteins.

FIG. 36A-36N shows the sense sequence (from: 1 to: 7056) and complement isolate 1078 as well as a translated amino acid sequence.

### DETAILED DESCRIPTION

The issued patents, applications, and other publications that are cited herein are hereby incorporated by reference to the same extent as if each was specifically and individually indicated to be incorporated by reference.

As used herein, “HRV-NY” refers to isolates of the new picornaviruses provided by the invention.

Rapid differential diagnosis of infectious diseases is a significant challenge in medicine and public health. During a period of a high incidence of influenza-like illness (ILI) reported in New York State, many samples tested negative for influenza virus by molecular testing, and negative for other respiratory viruses by culture. This indicated that a previously unidentified agent might be implicated in the cause of influenza like-disease.

In certain aspects the invention provides a multiplex diagnostic tool such as MassTag PCR for detection of respiratory pathogens. In certain embodiments, the invention provides isolation and nucleic acids sequences derived from a new rhinovirus genotype.

In certain aspects, the invention provides that implementation of MassTag PCR detection can identify pathogenic agents in samples negative for known respiratory viruses. In non-limiting example, implementation of MassTag PCR resolved 26 of 79 previously negative samples, revealing the presence of rhinoviruses in a large proportion of samples, half of which belonged to a previously uncharacterized genetic clade. In certain embodiments, knowledge of the detected viral and/or bacterial (co-)infection can provide altered clinical management.

An inexpensive, sensitive, highly multiplexed system for differential diagnosis of infectious diseases was described (Briese et al., 2005). See also U.S. patent application Ser. No. 11/119,231, the contents of which are hereby incorporated by reference. This MassTag polymerase chain reaction (PCR) platform detects 22 different respiratory viral and bacterial pathogens and has been evaluated with cultured isolates and respiratory specimens from infected individuals. Recently, MassTag PCR has also been applied to the detection of agents causing viral hemorrhagic fevers in specimens available from patients with Ebola, Marburg, Lassa, Rift Valley, and Crimean-Congo hemorrhagic fever (Palacios et al., 2006).

During a period of a high incidence in New York State of ILI, multiple samples tested negative for influenza virus A (FLUAV) and B (FLUBV) by real-time reverse transcription (RT)-PCR, and were also negative for other respiratory viruses by conventional virus culture. Absence of correlation between ILI and known pathogens indicated that there is previously unidentified agent causing the illness, which led to analyses using the MassTag PCR platform (Briese et al., 2005).

During the 2004-2005 influenza season, the New York State Department of Health received 166 samples through the Influenza Sentinel Physicians Surveillance Network, 151 of which were analyzed. Samples were analyzed on arrival in the laboratory using antigen detection and real-time RT-PCR assays designed to identify influenza viruses, as well as conventional virus culture for the detection of additional respiratory viral pathogens. FLUAV (n=58) and FLUBV (n=10) accounted for the majority of the identified agents; in addition, two HADV, one HPIV-1, and two HSV were isolated. In one of the samples, co-infection with FLUAV and FLUBV was demonstrated. Overall, these analyses identified 73 agents in 72 samples, providing a presumptive diagnosis in 48% of cases. However, 79 samples remained without an identified pathogen. Some samples were collected more than 10 days after onset of symptoms; thus, low microbial load at time of collection could have accounted for some of the negative results. However, many of these negative samples were clustered during October to December of 2004 and were indicative of the presence of an unidentified agent. All available stored specimens were retrospectively investigated by multiplex MassTag PCR to identify agent(s) which caused ILI.

Analysis of the retrospective samples by MassTag PCR indicated the presence of 109 agents in 93 samples and identified a pathogen in 33% of the previously negative specimens (Table 1a). In the 26 cases that lacked a previous diagnosis, 33 agents were detected. In 8 of the samples for which an agent had been previously identified, MassTag PCR revealed the additional presence of 9 other agents (Table 1b). Furthermore, MassTag PCR revealed infection with 2 agents in each of 9 patients, and with 3 agents in each of 4 patients. This study confirms the utility of MassTag PCR as a tool for surveillance, outbreak detection and epidemiology. It's potential to rapidly query samples for the presence of a wide range of candidate viral and bacterial pathogens that may act alone or in concert suggests that MassTag PCR can also have applications in clinical medicine.

Results obtained with real-time RT-PCR and MassTag PCR assays were in 100% accord for HADV, HPIV-1, and FLUBV; there was 96% accord for FLUAV. Results obtained with real-time RT-PCR and MassTag PCR were discordant for 2 FLUAV real-time RT-PCR positive samples. The viral load in these samples, as indicated by the real-time RT-PCR Ct values, was less than 1000 RNA copies per reaction, which is below the detection limit determined for FLUAV in MassTag PCR.

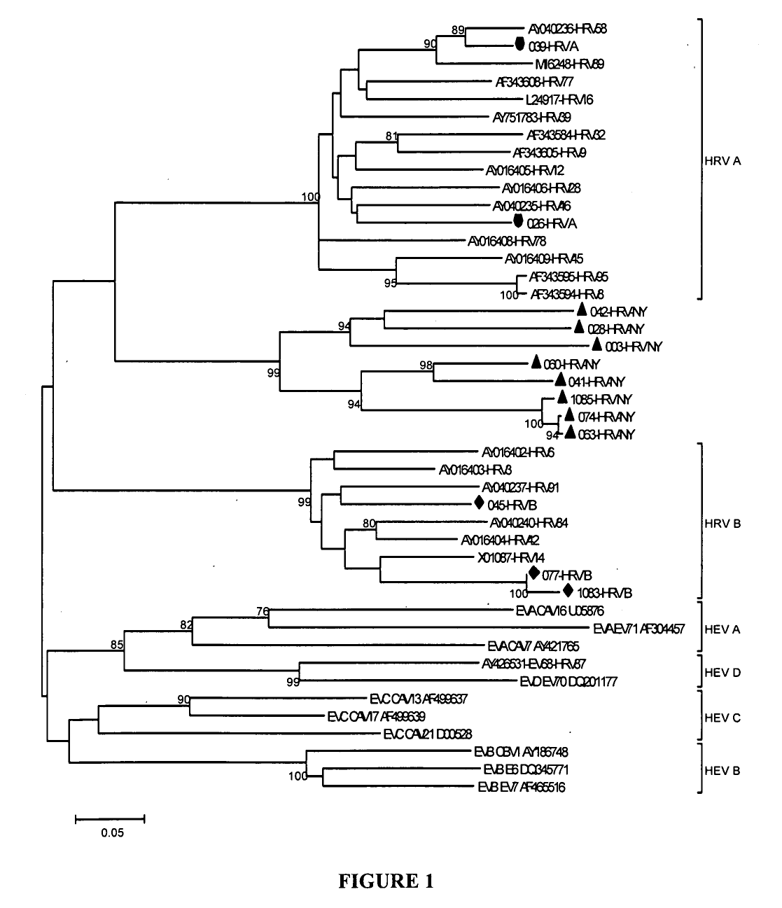

The degenerate HEV primers used in the MassTag PCR assay target conserved regions in the 5′-UTR of picornaviruses that are also present in human rhinoviruses (HRV). When samples that had tested positive with this primer pair were tested with a specific diagnostic real-time RT-PCR assay for HEV, 17 of the 18 cases yielded a negative result. All MassTag PCR amplification products were cloned, based on the reasoning that products represented either novel HEV or HRV isolates. Sequence analysis identified 2 HEVs and 16 HRVs (Table 1a and b). The 16 HRV sequences were most closely related to a mixed population of HEV and HRV 5‘-UTRs listed in GenBank as ‘Antwerp rhinovirus’ (Loens et al., 2006). Because short 5′-UTR sequences are not suitable for assignment of phylogenetic relationships, additional sequence using degenerate primer sets targeting the VP4 gene region were obtained (Coiras et al., 2004). Phylogenetic analysis of VP4 sequences indicated HRV group A in 2 cases and HRV group B in 3 cases (table 1a and b); in 3 instances, the sample was exhausted before an amplification product was obtained. For the 8 other cases, sequences clustered in a clade at the root of HRV group A, distinct from the described group A or B serotypes (Horsnell et al., 1995) (FIG. 1). In one case, designated as specimen 074, additional analysis using a highly degenerate primer set (Nix et al., in press) allowed amplification of partial VP1 sequence; the analysis supported the phylogenetic position indicated by VP4 analysis.

The analyses described herein were undertaken to investigate the causes of ILI in New York State between Oct. 1, 2004 and May 31, 2005. Clinical samples obtained by physicians in a surveillance network were submitted for analysis by a standard diagnostic protocol using viral culture, antigen detection, and molecular assays. In addition to the agents identified by these methods, MassTag PCR detected HRV, HEV, S. pneumoniae, M. pneumoniae, H. influenzae, HMPV, HCoV-OC 43, RSV-A, HPIV-1, and N. meningitidis infections. MassTag PCR also revealed instances in which virus-infected ILI patients were co-infected with potentially treatable bacterial pathogens.

In certain aspects, the invention provides that ILI is associated with a high incidence of rhinovirus infection. Although rhinoviruses are most commonly associated with mild upper respiratory disease, they have also been described in association with severe acute and lower respiratory tract infections in children, the elderly, and immunosuppressed patients.

The present invention provides picornavirus nucleic acid sequences. These nucleic acid sequences may be useful for, inter alia, expression of picornavirus-encoded proteins or fragments, variants, or derivatives thereof, generation of antibodies against picornavirus proteins, generation of primers and probes for detecting picornaviruses and/or for diagnosing picornavirus infection, generating vaccines against picornaviruses, and screening for drugs effective against picornaviruses, as described below.

In certain aspects, the invention is directed to a rhinovirus isolated nucleic acid sequence as provided in any one of SEQ ID NO: 1-23. The rhinovirus nucleic acids sequences as provided in any one of SEQ ID NO: 1-23 were identified from 8 cases of ILI that clustered during an 8-week period from October to December 2004. Thus, the invention provides that rhinoviruses are a major cause of ILI.

In vitro evidence indicates that HRV infection may enhance the probability for streptococcal infection, through up-regulation of the platelet-activating factor receptor (Ishizuka et al, 2003). Whether the 3 cases of co-infection between HRV and S. pneumoniae observed in this study reflect a similar interaction between the two agents remains to be determined. More comprehensive data are required before the role of multiple infections in the pathogenesis of respiratory, or other, diseases can be assessed.

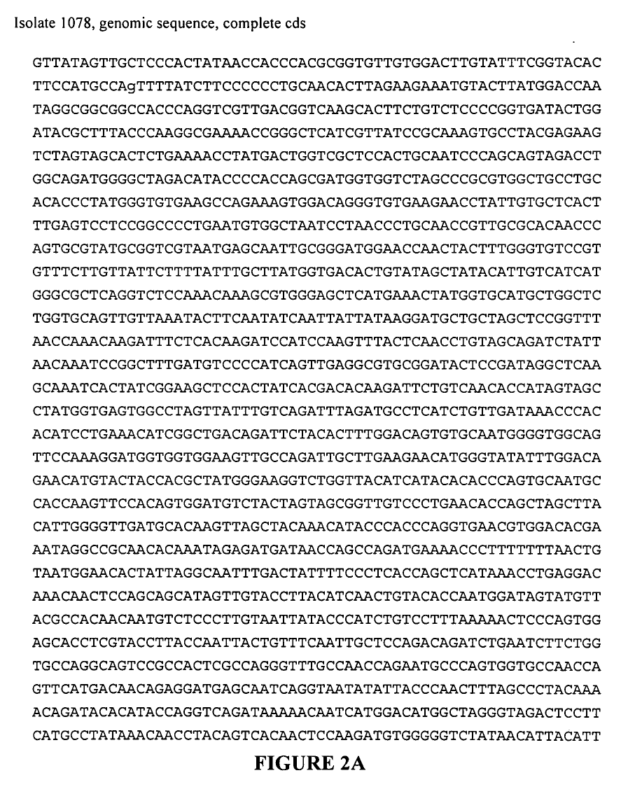

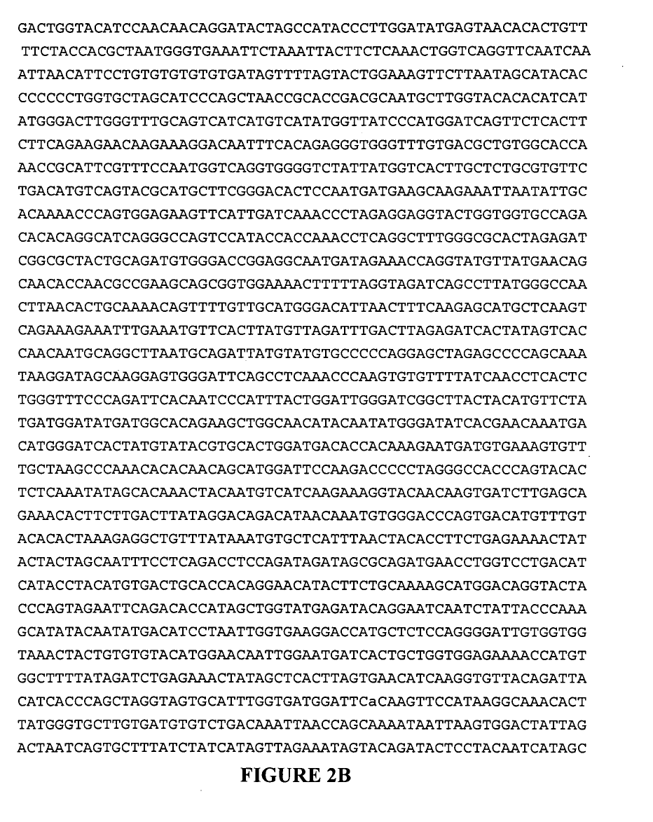

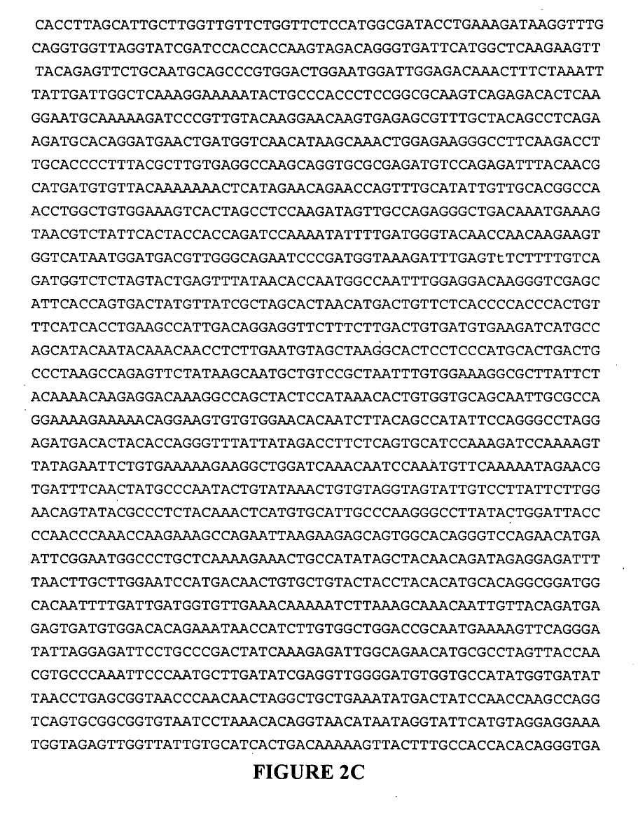

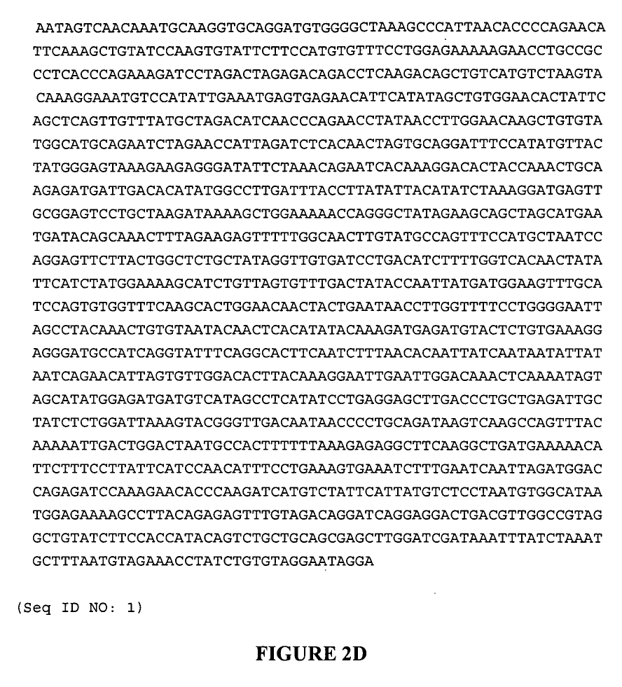

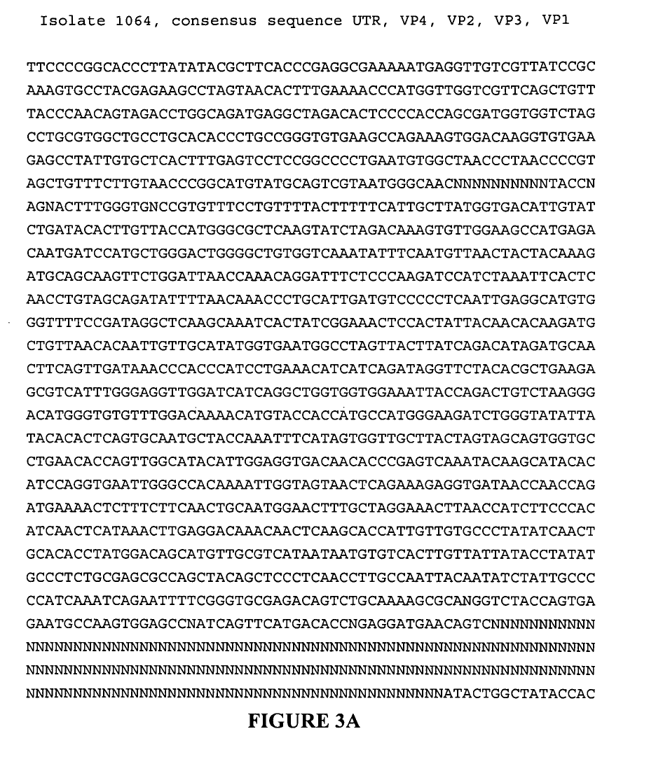

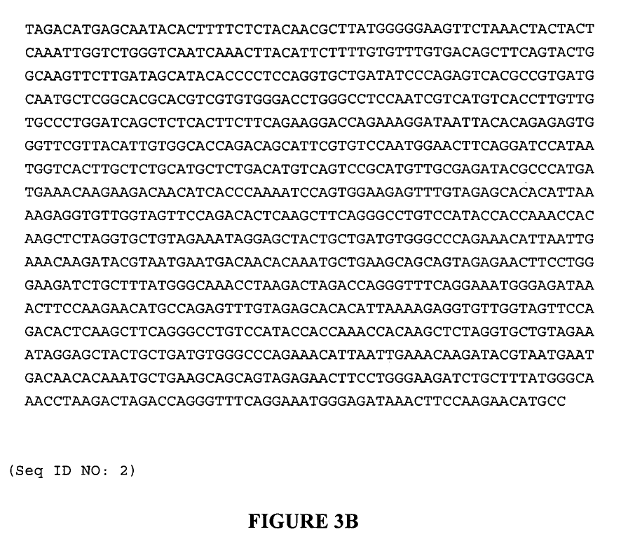

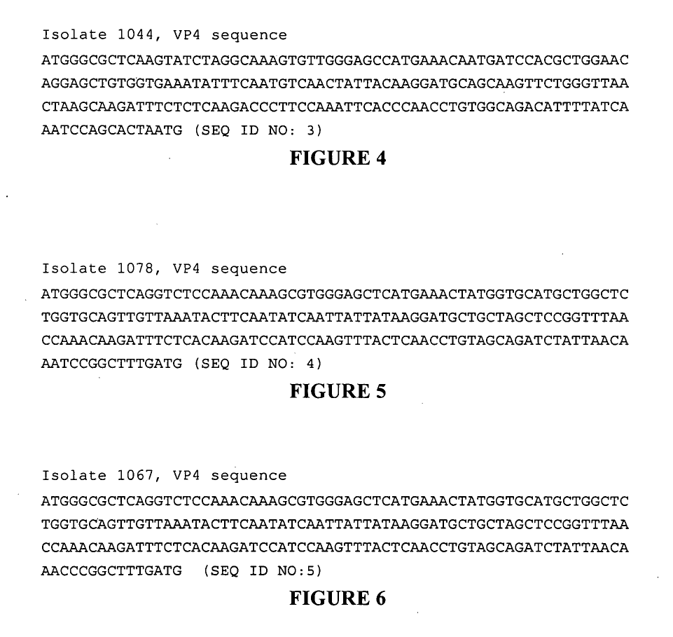

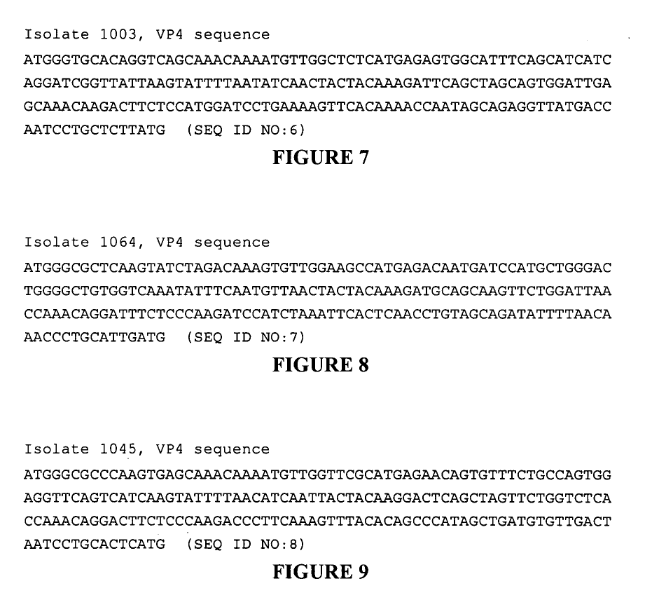

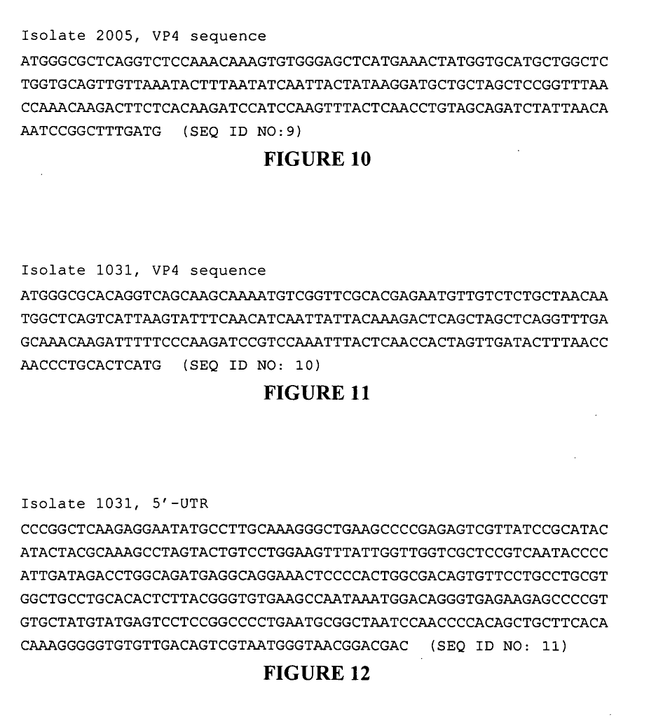

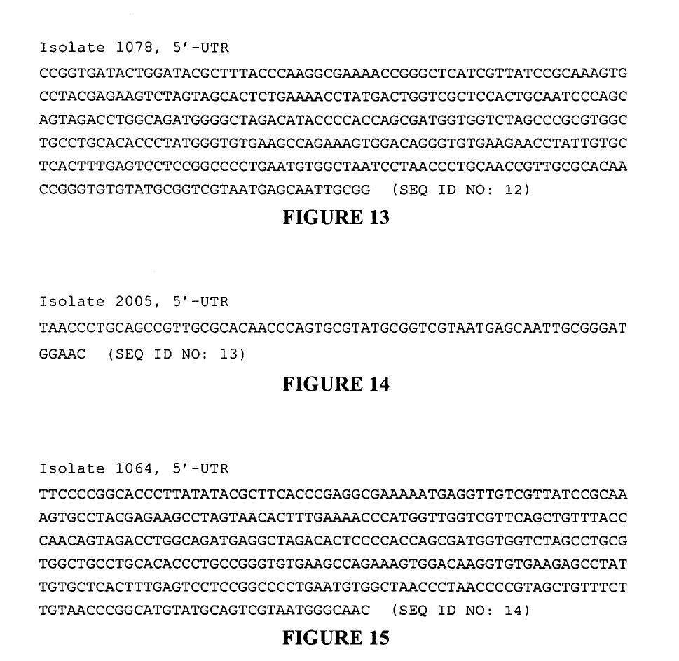

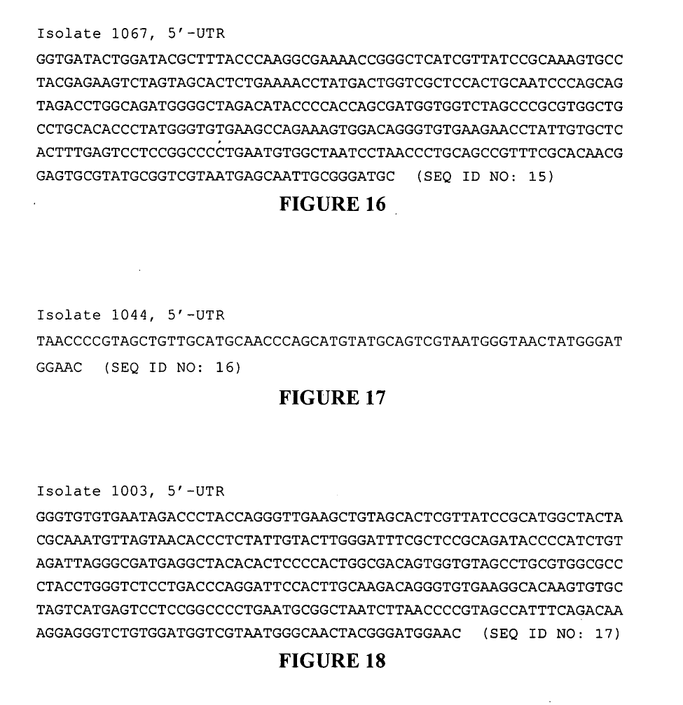

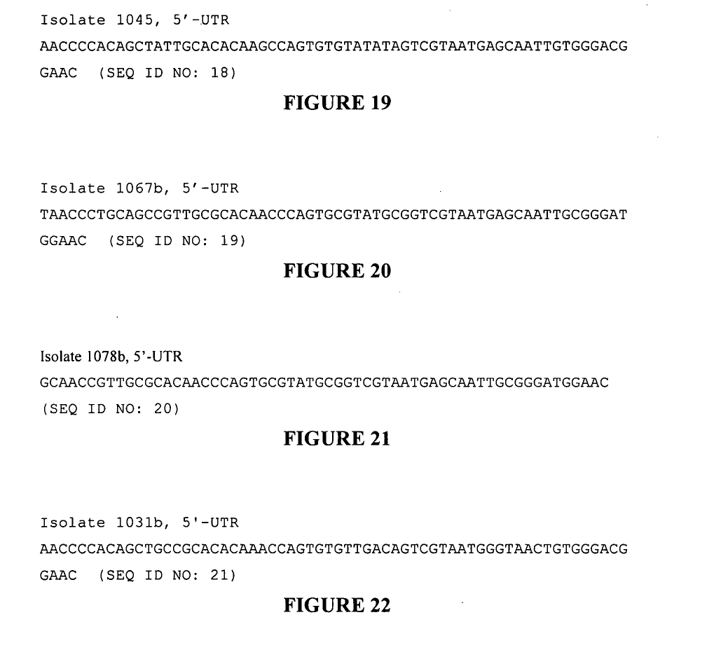

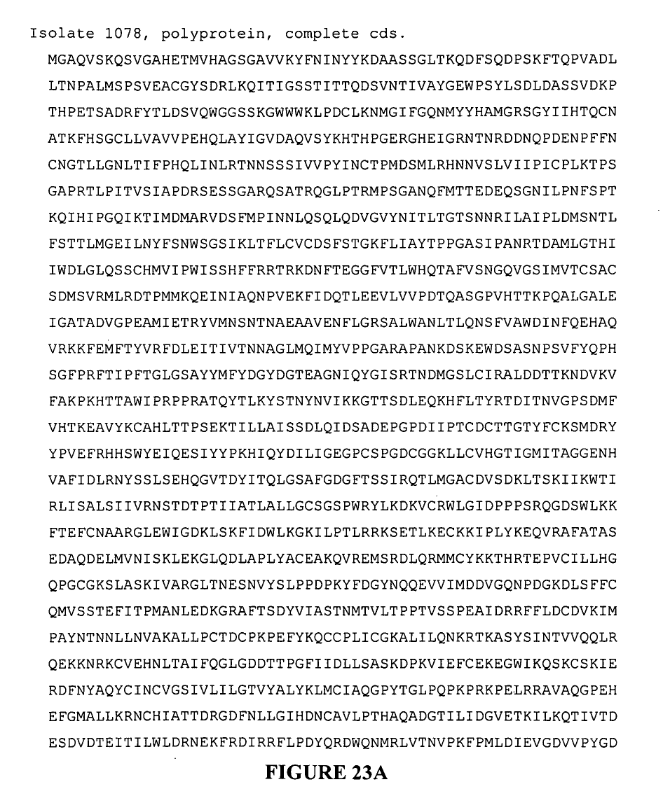

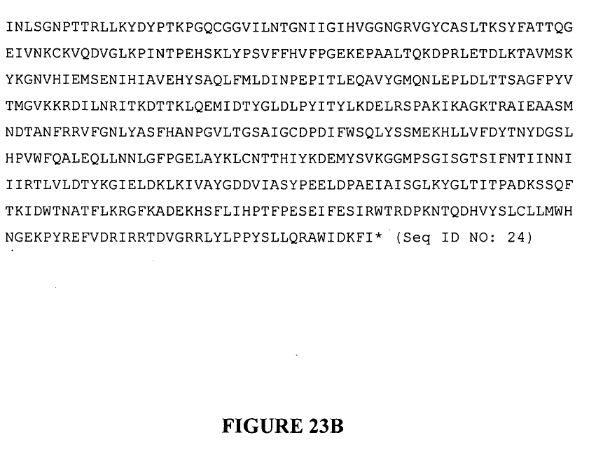

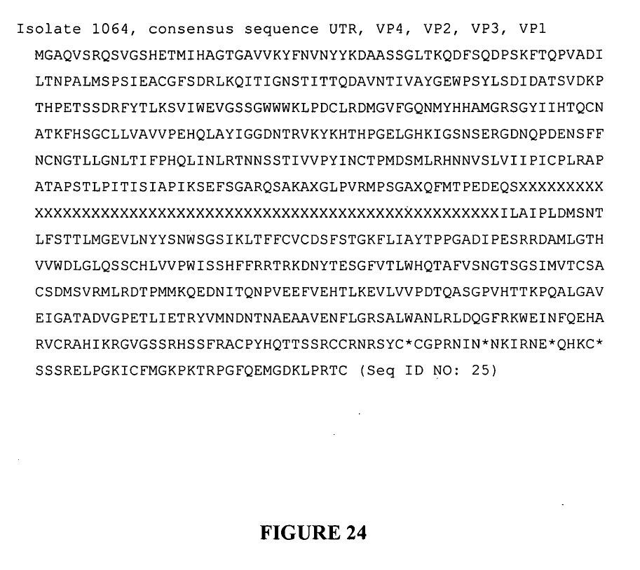

In certain aspects, the invention is directed to an isolated nucleic acid of any one of SEQ ID NO: 1-23. SEQ ID NO: 1-23 are listed in FIGS. 2-25. The invention is directed to an isolated nucleic acid complementary to any one of SEQ ID NO: 1-23.

In certain aspects, the invention is directed to isolated nucleic acid sequence variants of any one of SEQ ID NO: 1-23. Contemplated variants of SEQ ID NO: 1-23 include but are not limited to nucleic acid sequences having at least from about 50% to about 55% identity to that of SEQ ID NO: 1-23. Contemplated variants of SEQ ID NO: 1-23 include but are not limited to nucleic acid sequences having at least from about 55.1% to about 60% identity to that of SEQ ID NO: 1-23. Contemplated variants of SEQ ID NO: 1-23 include but are not limited to nucleic acid sequences having at least from about 60.1% to about 65% identity to that of SEQ ID NO: 1-23. Contemplated variants of SEQ ID NO: 1 include but are not limited to nucleic acid sequences having at least from about 65.1% to about 70% identity to that of SEQ ID NO: 1-23. Contemplated variants of SEQ ID NO:1 include but are not limited to nucleic acid sequences having at least from about 70.1% to about 75% identity to that of SEQ ID NO: 1-23. Contemplated variants of SEQ ID NO: 1-23 include but are not limited to nucleic acid sequences having at least from about 75.1% to about 80% identity to that of SEQ ID NO: 1-23. Contemplated variants of SEQ ID NO: 1-23 include but are not limited to nucleic acid sequences having at least from about 80.1% to about 85% identity to that of SEQ ID NO: 1-23. Contemplated variants of SEQ ID NO: 1-23 include but are not limited to nucleic acid sequences having at least from about 85.1% to about 90% identity to that of SEQ ID NO: 1-23. Contemplated variants of SEQ ID NO:1-23 include but are not limited to nucleic acid sequences having at least from about 90.1% to about 95% identity to that of SEQ ID NO: 1-23. Contemplated variants of SEQ ID NO: 1-23 include but are not limited to nucleic acid sequences having at least from about 95.1% to about 97% identity to that of SEQ ID NO: 1-23. Contemplated variants of SEQ ID NO:1-23 include but are not limited to nucleic acid sequences having at least from about 97.1% to about 99% identity to that of SEQ ID NO: 1-23. Programs and algorithms for sequence alignment and comparison of % identity and/or homology between nucleic acid sequences, or polypeptides, are well known in the art, and include BLAST, SIM alignment tool, and so forth.

The invention is directed to an isolated nucleic acid sequence comprising from about 10 to about 50 consecutive nucleotides from any one of SEQ ID NO: 1-23 or a sequence complementary to any one of SEQ ID NO: 1-23. The invention is directed to an isolated nucleic acid sequence comprising from about 10 to about 100 consecutive nucleotides from any one of SEQ ID NO: 1-23 or a sequence complementary to any one of SEQ ID NO: 1-23. The invention is directed to an isolated nucleic acid sequence comprising from about 10 to about 200 consecutive nucleotides from any one of SEQ ID NO: 1-23 or a sequence complementary to any one of SEQ ID NO: 1-23. The invention is directed to an isolated nucleic acid sequence comprising from about 10 to about 300 consecutive nucleotides from any one of SEQ ID NO: 1-23 or a sequence complementary to any one of SEQ ID NO: 1-23. The invention is directed to an isolated nucleic acid sequence comprising from about 10 to about 400 consecutive nucleotides from SEQ ID NO: 1 or a sequence complementary to any one of SEQ ID NO: 1-23. The invention is directed to an isolated nucleic acid sequence comprising from about 10 to about 500 consecutive nucleotides from any one of SEQ ID NO: 1-23 or a sequence complementary to any one of SEQ ID NO: 1-23. The invention is directed to an isolated nucleic acid sequence comprising from about 10 to about 1000 consecutive nucleotides from any one of SEQ ID NO: 1-23 or a sequence complementary to any one of SEQ ID NO: 1-23. The invention is directed to an isolated nucleic acid sequence comprising from about 10 to about 1400 consecutive nucleotides from any one of SEQ ID NO: 1-23 or a sequence complementary to any one of SEQ ID NO: 1-23. The invention is directed to an isolated nucleic acid sequence comprising from about 10 to about 2000 consecutive nucleotides from any one of SEQ ID NO: 1-23 or a sequence complementary to any one of SEQ ID NO: 1-23. The invention is directed to an isolated nucleic acid sequence comprising from about 10 to about 2400 consecutive nucleotides from any one of SEQ ID NO: 1-23 or a sequence complementary to any one of SEQ ID NO: 1-23. The invention is directed to an isolated nucleic acid sequence comprising from about 10 to about 2700 consecutive nucleotides from any one of SEQ ID NO: 1-23 or a sequence complementary to any one of SEQ ID NO: 1-23. The invention is directed to an isolated nucleic acid sequence comprising from about 10 to about 2900 consecutive nucleotides from any one of SEQ ID NO: 1-23 or a sequence complementary to any one of SEQ ID NO: 1-23.

In other aspects the invention is directed to isolated nucleic acid sequences such as primers and probes, comprising nucleic acid sequences derived from any one of SEQ ID NO: 1-23. Such primers and/or probes may be useful for detecting the presence of the picornaviruses of the invention, for example in samples of bodily fluids such as blood, saliva, or urine from a subject, and thus may be useful in the diagnosis of picornavirus infection. Such probes can detect polynucleotides of SEQ ID NO: 1-23 in samples which comprise picornaviruses represented by SEQ ID NO: 1-23. The isolated nucleic acids which can be used as primer and/probes are of sufficient length to allow hybridization with, i.e. formation of duplex with a corresponding target nucleic acid sequence, a nucleic acid sequences of any one of SEQ ID NO: 1-23, or a variant thereof.

The isolated nucleic acid of the invention which can be used as primers and/or probes can comprise about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 consecutive nucleotides from any one of SEQ ID NO: 1-23, or sequences complementary to any one of SEQ ID NO: 1-23. The isolated nucleic acid of the invention which can be used as primers and/or probes can comprise from about 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 and up to about 20, 25, 30, 35, 40, 45, 50, 55, 60, 65, 70, 75, 80, 85, 90, 95 and 100 consecutive nucleotides from any one of SEQ ID NO: 1-23, or sequences complementary to any one of SEQ ID NO: 1-23. The invention is also directed to primer and/or probes which can be labeled by any suitable molecule and/or label known in the art, for example but not limited to fluorescent tags suitable for use in Real Time PCR amplification, for example TaqMan™, cybergreen, TAMRA and/or FAM probes; radiolabels, and so forth. In certain embodiments, the oligonucleotide primers and/or probe further comprises a detectable non-isotopic label selected from the group consisting of: a fluorescent molecule, a chemiluminescent molecule, an enzyme, a cofactor, an enzyme substrate, and a hapten.

In certain aspects, the invention is directed to primer sets comprising isolated nucleic acids as described herein, which primer set are suitable for amplification of nucleic acids from samples which comprises picornaviruses represented by any one of SEQ ID NO: 1-23, or variants thereof. Primer sets can comprise any suitable combination of primers which would allow amplification of a target nucleic acid sequences in a sample which comprises picornaviruses represented by any one of SEQ ID NO: 1-23, or variants thereof. Amplification can be performed by any suitable method known in the art, for example but not limited to PCR, RT-PCR, transcription mediated amplification (TMA).

For example, the nucleic acids described herein represented by any one of SEQ ID NO: 1-23, or variants thereof can be used with any method described herein suitable for detecting the presence or absence of the novel picornavirus in a biological sample. In one embodiment, the method can comprise contacting nucleic acid from a biological sample with at least one primer which is a synthetic nucleic acid which has a sequence consisting of from about 10 to about 30 consecutive nucleotides from a nucleic acids sequence selected from the group of sequences consisting of SEQ ID NO: 1-23, subjecting the nucleic acid and the primer to amplification conditions, and determining the presence or absence of amplification product, wherein the presence of amplification product indicates the presence of RNA associated with picornavirus in the sample. For example, the nucleic acids described herein are suitable for detecting the presence or absence of picornaviruses in a sample, for example, see Briese et al., 2008; Dominguez et al., 2008 and Renwick et al., 2007—each of which is incorporated in their entirety and any sequences cited therein are incorporated by reference to the same extent as if each was specifically and individually indicated to be incorporated by reference.

Hybridization Conditions

As used herein, the phrase “stringent hybridization conditions” refers to conditions under which a probe, primer or oligonucleotide will hybridize to its target sequence, and can hybridize, for example but not limited to, variants of the disclosed polynucleotide sequences, including allelic or splice variants, or sequences that encode orthologs or paralogs of presently disclosed polypeptides. The precise conditions for stringent hybridization are typically sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Nucleic acid hybridization methods are disclosed in detail by Kashima et al. (1985) Nature 313:402-404, and Sambrook et al. (1989) Molecular Cloning: A Laboratory Manual, 2nd Ed., Cold Spring Harbor Laboratory, Cold Spring Harbor, N.Y (“Sambrook”); and by Haymes et al., “Nucleic Acid Hybridization: A Practical Approach”, IRL Press, Washington, D.C. (1985), which references are incorporated herein by reference.

In general, stringency is determined by the temperature, ionic strength, and concentration of denaturing agents (e.g., formamide) used in a hybridization and washing procedure. The degree to which two nucleic acids hybridize under various conditions of stringency is correlated with the extent of their similarity. Numerous variations are possible in the conditions and means by which nucleic acid hybridization can be performed to isolate nucleic sequences having similarity to the nucleic acid sequences known in the art and are not limited to those explicitly disclosed herein. Such an approach may be used to isolate polynucleotide sequences having various degrees of similarity with disclosed nucleic acid sequences, such as, for example, nucleic acid sequences having 60% identity, or about 70% identity, or about 80% or greater identity with disclosed nucleic acid sequences.

Stringent conditions are known to those skilled in the art and can be found in Current Protocols In Molecular Biology, John Wiley & Sons, N.Y. (1989), 6.3.1-6.3.6. In certain embodiments, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6× sodium chloride/sodium citrate (SSC), 50 mM Tris-HCl (pH 7.5), 1 nM EDTA, 0.02% PVP, 0.02% Ficoll, 0.02% BSA, and 500 mg/ml denatured salmon sperm DNA at 65° C. This hybridization is followed by one or more washes in 0.2×SSC, 0.01% BSA at 50° C. Another non-limiting example of stringent hybridization conditions are hybridization in 6× sodium chloride/sodium citrate (SSC) at about 45° C., followed by one or more washes in 0.2×SSC, 0.1% SDS at 50-65° C. Examples of moderate to low stringency hybridization conditions are well known in the art.

Polynucleotides homologous to the sequences illustrated in the Sequence Listing and figures can be identified, e.g., by hybridization to each other under stringent or under highly stringent conditions. Single stranded polynucleotides hybridize when they associate based on a variety of well characterized physical-chemical forces, such as hydrogen bonding, solvent exclusion, base stacking and the like. The stringency of a hybridization reflects the degree of sequence identity of the nucleic acids involved, such that the higher the stringency, the more similar are the two polynucleotide strands. Stringency is influenced by a variety of factors, including temperature, salt concentration and composition, organic and non-organic additives, solvents, etc. present in both the hybridization and wash solutions and incubations (and number thereof, as described in more detail in the references cited above.

Encompassed by the invention are polynucleotide sequences that are capable of hybridizing to the claimed polynucleotide sequences, including any of the nucleic acid sequences disclosed herein, and fragments thereof under various conditions of stringency (See, for example, Wahl and Berger (1987) Methods Enzymol. 152: 399-407; and Kimmel (1987) Methods Enzymol. 152: 507-511). With regard to hybridization, conditions that are highly stringent, and means for achieving them, are well known in the art. See, for example, Sambrook et al. (1989) “Molecular Cloning: A Laboratory Manual” (2nd ed., Cold Spring Harbor Laboratory); Berger and Kimmel, eds., (1987) “Guide to Molecular Cloning Techniques”, In Methods in Enzymology: 152: 467-469; and Anderson and Young (1985) “Quantitative Filter Hybridisation.” In: Hames and Higgins, ed., Nucleic Acid Hybridisation, A Practical Approach. Oxford, IRL Press, 73-111.

Stability of DNA duplexes is affected by such factors as base composition, length, and degree of base pair mismatch. Hybridization conditions may be adjusted to allow DNAs of different sequence relatedness to hybridize. The melting temperature (Tm) is defined as the temperature when 50% of the duplex molecules have dissociated into their constituent single strands. The melting temperature of a perfectly matched duplex, where the hybridization buffer contains formamide as a denaturing agent, may be estimated by the following equation: DNA-DNA: Tm(° C.)=81.5+16.6(log [Na+])+0.41(% G+C)−0.62(% formamide)−500/L (1) DNA-RNA: Tm(° C.)=79.8+18.5(log [Na+])+0.58(% G+C)+0.12(% G+C).sup.2−0.5(% formamide)−820/L (2) RNA-RNA: Tm(C)=79.8+18.5(log [Na+])+0.58(% G+C)+0.12(% G+C).sup.2−0.35(% formamide)−820/L (3), where L is the length of the duplex formed, [Na+] is the molar concentration of the sodium ion in the hybridization or washing solution, and % G+C is the percentage of (guanine+cytosine) bases in the hybrid. For imperfectly matched hybrids, approximately 1° C. is required to reduce the melting temperature for each 1% mismatch.

Hybridization experiments are generally conducted in a buffer of pH between 6.8 to 7.4, although the rate of hybridization is nearly independent of pH at ionic strengths likely to be used in the hybridization buffer (Anderson et al. (1985) supra). In addition, one or more of the following may be used to reduce non-specific hybridization: sonicated salmon sperm DNA or another non-complementary DNA, bovine serum albumin, sodium pyrophosphate, sodium dodecylsulfate (SDS), polyvinyl-pyrrolidone, ficoll and Denhardt's solution. Dextran sulfate and polyethylene glycol 6000 act to exclude DNA from solution, thus raising the effective probe DNA concentration and the hybridization signal within a given unit of time. In some instances, conditions of even greater stringency may be desirable or required to reduce non-specific and/or background hybridization. These conditions may be created with the use of higher temperature, lower ionic strength and higher concentration of a denaturing agent such as formamide.

Stringency conditions can be adjusted to screen for moderately similar fragments such as homologous sequences from distantly related organisms, or to highly similar fragments. The stringency can be adjusted either during the hybridization step or in the post-hybridization washes. Salt concentration, formamide concentration, hybridization temperature and probe lengths are variables that can be used to alter stringency (as described by the formula above). As a general guidelines high stringency is typically performed at Tm-5° C. to Tm-20° C., moderate stringency at Tm-20° C. to Tm-35° C. and low stringency at Tm-35° SC to Tm-50° C. for duplex>150 base pairs. Hybridization may be performed at low to moderate stringency (25-50° C. below T.sub.m), followed by post-hybridization washes at increasing stringencies. Maximum rates of hybridization in solution are determined empirically to occur at Tm-25° C. for DNA-DNA duplex and Tm-15° C. for RNA-DNA duplex. Optionally, the degree of dissociation may be assessed after each wash step to determine the need for subsequent, higher stringency wash steps.

High stringency conditions may be used to select for nucleic acid sequences with high degrees of identity to the disclosed sequences. An example of stringent hybridization conditions obtained in a filter-based method such as a Southern or northern blot for hybridization of complementary nucleic acids that have more than 100 complementary residues is about 5° C. to 20° C. lower than the thermal melting point (Tm) for the specific sequence at a defined ionic strength and pH. Conditions used for hybridization may include about 0.02 M to about 0.15 M sodium chloride, about 0.5% to about 5% casein, about 0.02% SDS or about 0.1% N-laurylsarcosine, about 0.001 M to about 0.03 M sodium citrate, at hybridization temperatures between about 50° C. and about 70° C. In certain embodiments, high stringency conditions are about 0.02 M sodium chloride, about 0.5% casein, about 0.02% SDS, about 0.001 M sodium citrate, at a temperature of about 50° C. Nucleic acid molecules that hybridize under stringent conditions will typically hybridize to a probe based on either the entire DNA molecule or selected portions, e.g., to a unique subsequence, of the DNA.

Stringent salt concentration will ordinarily be less than about 750 mM NaCl and 75 mM trisodium citrate. Increasingly stringent conditions may be obtained with less than about 500 mM NaCl and 50 mM trisodium citrate, to even greater stringency with less than about 250 mM NaCl and 25 mM trisodium citrate. Low stringency hybridization can be obtained in the absence of organic solvent, e.g., formamide, whereas in certain embodiments high stringency hybridization may be obtained in the presence of at least about 35% formamide, and in other embodiments in the presence of at least about 50% formamide. In certain embodiments, stringent temperature conditions will ordinarily include temperatures of at least about 30° C., and in other embodiment at least about 37° C., and in other embodiments at least about 42° C. with formamide present. Varying additional parameters, such as hybridization time, the concentration of detergent, e.g., sodium dodecyl sulfate (SDS) and ionic strength, are well known to those skilled in the art. Various levels of stringency are accomplished by combining these various conditions as needed. In a certain embodiment, hybridization will occur at 30° C. in 750 mM NaCl, 75 mM trisodium citrate, and 1% SDS. In another embodiment, hybridization will occur at 37° C. in 500 mM NaCl, 50 mM trisodium citrate, 1% SDS, 35% formamide. In another embodiment, hybridization will occur at 42C in 250 mM NaCl, 25 mM trisodium citrate, 1% SDS, 50% formamide. Useful variations on these conditions will be readily apparent to those skilled in the art.

The washing steps that follow hybridization may also vary in stringency; the post-hybridization wash steps primarily determine hybridization specificity, with the most critical factors being temperature and the ionic strength of the final wash solution. Wash stringency can be increased by decreasing salt concentration or by increasing temperature. Stringent salt concentration for the wash steps can be less than about 30 mM NaCl and 3 mM trisodium citrate, and in certain embodiments less than about 15 mM NaCl and 1.5 mM trisodium citrate. For example, the wash conditions may be under conditions of 01×SSC to 2.0×SSC and 0.1% SDS at 50-65° C., with, for example, two steps of 10-30 min. One example of stringent wash conditions includes about 2.0×SSC, 0.1% SDS at 65° C. and washing twice, each wash step being about 30 min. The temperature for the wash solutions will ordinarily be at least about 25° C., and for greater stringency at least about 42° C. Hybridization stringency may be increased further by using the same conditions as in the hybridization steps, with the wash temperature raised about 3° C. to about 5C, and stringency may be increased even further by using the same conditions except the wash temperature is raised about 6° C. to about 9° C. For identification of less closely related homolog, wash steps may be performed at a lower temperature, e.g., 50° C.

An example of a low stringency wash step employs a solution and conditions of at least 25° C. in 30 mM NaCl, 3 mM trisodium citrate, and 0.1% SDS over 30 min. Greater stringency may be obtained at 42° C. in 15 mM NaCl, with 1.5 mM trisodium citrate, and 0.1% SDS over 30 min. Even higher stringency wash conditions are obtained at 65° C.-68° C. in a solution of 15 mM NaCl, 1.5 mM trisodium citrate, and 0.1% SDS. Wash procedures will generally employ at least two final wash steps. Additional variations on these conditions will be readily apparent to those skilled in the art.

Stringency conditions can be selected such that an oligonucleotide that is perfectly complementary to the coding oligonucleotide hybridizes to the coding oligonucleotide with at least about a 5-10× higher signal to noise ratio than the ratio for hybridization of the perfectly complementary oligonucleotide to a nucleic acid. It may be desirable to select conditions for a particular assay such that a higher signal to noise ratio, that is, about 15× or more, is obtained. Accordingly, a subject nucleic acid will hybridize to a unique coding oligonucleotide with at least a 2× or greater signal to noise ratio as compared to hybridization of the coding oligonucleotide to a nucleic acid encoding known polypeptide. The particular signal will depend on the label used in the relevant assay, e.g., a fluorescent label, a calorimetric label, a radioactive label, or the like. Labeled hybridization or PCR probes for detecting related polynucleotide sequences may be produced by oligolabeling, nick translation, end-labeling, or PCR amplification using a labeled nucleotide.

The sequence identities can be determined by analysis with a sequence comparison algorithm or by a visual inspection. Protein and/or nucleic acid sequence identities (homologies) can be evaluated using any of the variety of sequence comparison algorithms and programs known in the art.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.2.2. or FASTA version 3.0t78 algorithms and the default parameters discussed below can be used.

A “comparison window”, as used herein, includes reference to a segment of any one of the number of contiguous positions selected from the group consisting of from 20 to 600, usually about 50 to about 200, more usually about 100 to about 150 in which a sequence can be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2: 482, 1981, by the homology alignment algorithm of Needleman & Wunsch, J. Mol. Biol. 48: 443, 1970, by the search for similarity method of Pearson & Lipman, Proc. Natl. Acad. Sci. U.S.A. 85: 2444, 1988, by computerized implementations of these algorithms (FASTDB (Intelligenetics), BLAST (National Center for Biomedical Information), GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., (1999 Suppl.), Current Protocols in Molecular Biology, Greene Publishing Associates and Wiley Interscience, N.Y., 1987)

An example of an algorithm that is suitable for determining percent sequence identity and sequence similarity is the FASTA algorithm, which is described in Pearson, W. R. & Lipman, D. J., Proc. Natl. Acad. Sci. U.S.A. 85: 2444, 1988. See also W. R. Pearson, Methods Enzymol. 266: 227-258, 1996. Exemplary parameters used in a FASTA alignment of DNA sequences to calculate percent identity are optimized, BL50 Matrix 15: −5, k-tuple=2; joining penalty=40, optimization=28; gap penalty −12, gap length penalty=−2; and width=16.

Another example of algorithm that is suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10 μM=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. U.S.A. 89:10915, 1989) alignments (B) of 50, expectation (E) of 10 μM=5, N=−4, and a comparison of both strands.

The BLAST algorithm also performs a statistical analysis of the similarity between two sequences (see, e.g., Karlin & Altschul, Proc. Natl. Acad. Sci. U.S.A. 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, less than about 0.01, and less than about 0.001.

Another example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35:351-360, 1987. The method used is similar to the method described by Higgins & Sharp, CABIOS 5:151-153, 1989. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al, Nuc. Acids Res. 12:387-395, 1984.

Another example of an algorithm that is suitable for multiple DNA and amino acid sequence alignments is the CLUSTALW program (Thompson, J. D. et al., Nucl. Acids. Res. 22:4673-4680, 1994). ClustalW performs multiple pairwise comparisons between groups of sequences and assembles them into a multiple alignment based on homology. Gap open and Gap extension penalties were 10 and 0.05 respectively. For amino acid alignments, the BLOSUM algorithm can be used as a protein weight matrix (Henikoff and Henikoff, Proc. Natl. Acad. Sci. U.S.A. 89:10915-10919, 1992).

“Percent identity” in the context of two or more nucleic acids or polypeptide sequences, refers to the percentage of nucleotides or amino acids that two or more sequences or subsequences contain which are the same. A specified percentage of amino acid residues or nucleotides can be referred to such as: 60% identity, 65%, 70%, 75%, 80%, 85%, 90%, 91%, 92%, 93%, 94%, 95%, 96%, 97%, 98%, or 99% or more identity over a specified region, when compared and aligned for maximum correspondence over a comparison window, or designated region as measured using one of the following sequence comparison algorithms or by manual alignment and visual inspection.

“Substantially identical,” in the context of two nucleic acids or polypeptides, refers to two or more sequences or subsequences that have at least of at least 98%, at least 99% or higher nucleotide or amino acid residue identity, when compared and aligned for maximum correspondence, as measured using one of the following sequence comparison algorithms or by visual inspection.

In other aspects, the invention is directed to expression constructs, for example but not limited to plasmids and vectors which comprise nucleic acid sequences of SEQ ID NO: 1-10, complementary sequences thereof, and/or variants thereof. Such expression constructs can be prepared by any suitable method known in the art. Such expression constructs are suitable for viral nucleic acid and/or protein expression and purification.

The novel picornavirus shares less than 70% amino acid identity in P1 with: human rhinovirus A (highest identity [in BLAST search] is 50% with HRV 89, 39, 16, and 2; 49% with HRV 1B); human rhinovirus B (47% with HRV 14); human enterovirus B (46% E-16, SVDV(CV-B5); 45% E-4); human enterovirus C (45% with CV-A21, CV-A19); Human enterovirus D (43% with EV-70); and human enterovirus A (42% with CV-10, CV-8). The novel picornavirus of the invention shares less than 70% amino acid identity in the non-structural proteins 2C+3CD with: human rhinovirus A (highest identity [in ‘gap’ comparison] is 55% with HRV 39; 54% with HRV 89); and human rhinovirus B (53% with HRV 14); and 50-53% with HEVs or PVs.

In certain aspects, the invention is directed to iRNA molecules which target nucleic acids from picornaviruses, for example but not limited to SEQ ID NO: 1-10, and variants thereof, and silence a target gene.

An “iRNA agent” (abbreviation for “interfering RNA agent”) as used herein, is an RNA agent, which can down-regulate the expression of a target gene, e.g. a picornavirus gene. An iRNA agent may act by one or more of a number of mechanisms, including post-transcriptional cleavage of a target mRNA sometimes referred to in the art as RNAi, or pre-transcriptional or pre-translational mechanisms. An iRNA agent can be a double stranded (ds) iRNA agent.

A “ds iRNA agent” (abbreviation for “double stranded iRNA agent”), as used herein, is an iRNA agent which includes more than one, and in certain embodiments two, strands in which interchain hybridization can form a region of duplex structure. A “strand” herein refers to a contiguous sequence of nucleotides (including non-naturally occurring or modified nucleotides). The two or more strands may be, or each form a part of, separate molecules, or they may be covalently interconnected, e.g. by a linker, e.g. a polyethyleneglycol linker, to form but one molecule. At least one strand can include a region which is sufficiently complementary to a target RNA. Such strand is termed the “antisense strand”. A second strand comprised in the dsRNA agent which comprises a region complementary to the antisense strand is termed the “sense strand”. However, a ds iRNA agent can also be formed from a single RNA molecule which is, at least partly; self-complementary, forming, e.g., a hairpin or panhandle structure, including a duplex region. In such case, the term “strand” refers to one of the regions of the RNA molecule that is complementary to another region of the same RNA molecule.

Although, in mammalian cells, long ds iRNA agents can induce the interferon response which is frequently deleterious, short ds iRNA agents do not trigger the interferon response, at least not to an extent that is deleterious to the cell and/or host. The iRNA agents of the present invention include molecules which are sufficiently short that they do not trigger a deleterious interferon response in mammalian cells. Thus, the administration of a composition of an iRNA agent (e.g., formulated as described herein) to a mammalian cell can be used to silence expression of a picornavirus gene while circumventing a deleterious interferon response.

Molecules that are short enough that they do not trigger a deleterious interferon response are termed siRNA agents or siRNAs herein. “siRNA agent” or “siRNA” as used herein, refers to an iRNA agent, e.g., a ds iRNA agent, that is sufficiently short that it does not induce a deleterious interferon response in a human cell, e.g., it has a duplexed region of less than about 30 nucleotide pairs.

iRNA agents as described herein, including ds iRNA agents and siRNA agents, can mediate silencing of a gene, e.g., by RNA degradation. For convenience, such RNA is also referred to herein as the RNA to be silenced. Such a gene is also referred to as a target gene. In certain embodiments, the RNA to be silenced is a gene product of a picornavirus gene, for example but not limited to viral VP1, 2, 3, and 4 gene product.

As used herein, the phrase “mediates RNAi” refers to the ability of an agent to silence, in a sequence specific manner, a target gene. “Silencing a target gene” means the process whereby a cell containing and/or secreting a certain product of the target gene when not in contact with the agent, will contain and/or secret at least 10%, 20%, 30%, 40%, 50%, 60%, 70%, 80%, or 90% less of such gene product when contacted with the agent, as compared to a similar cell which has not been contacted with the agent. Such product of the target gene can, for example, be a messenger RNA (mRNA), a protein, or a regulatory element.

In the anti viral uses of the present invention, silencing of a target gene can result in a reduction in “viral titer” in the cell or in the subject, wherein “reduction in viral titer” refers to a decrease in the number of viable virus produced by a cell or found in an organism undergoing the silencing of a viral target gene. Reduction in the cellular amount of virus produced can lead to a decrease in the amount of measurable virus produced in the tissues of a subject undergoing treatment and a reduction in the severity of the symptoms of the viral infection. iRNA agents of the present invention are also referred to as “antiviral iRNA agents”.

As used herein, a “picornavirus gene” refers to any one of the genes identified in the picornavirus virus genome. These genes are known in the art, and for example include the genes that encode the VP1, 2, 3, and 4 proteins.

In other aspects, the invention provides methods for reducing viral titer in a subject, by administering to a subject, at least one iRNA which inhibits the expression of a picornavirus gene.

In other aspects, the invention provides methods for identifying and/or generating anti-viral drugs. For example, in one aspect the invention provides methods for identifying drugs that bind to and/or inhibit the function of the picornavirus-encoded proteins of the invention, or that inhibit the replication or pathogenicity of the picornaviruses of the invention. Methods of identifying drugs that affect or inhibit a particular drug target, such as high throughput drug screening methods, are well known in the art and can readily be applied to the proteins and viruses of the present invention.

Isolated Polypeptides

The invention is also directed to isolated polypeptides and variants and derivatives thereof. These polypeptides may be useful for multiple applications, including, but not limited to, generation of antibodies and generation of immunogenic compositions. For example, the invention is directed to an isolated polypeptide having the sequence of any one of SEQ ID NO: 24-35.

In one aspect, the invention is directed to polypeptide variants of any one of SEQ ID NO: 24-35. Contemplated variants of any one of SEQ ID NO: 24-35 include but are not limited to polypeptide sequences having at least from about 50% to about 55% identity to that of any one of SEQ ID NO: 24-35. Contemplated variants of any one of SEQ ID NO: 24-35 include but are not limited to polypeptide sequences having at least from about 55.1% to about 60% identity to that of any one of SEQ ID NO: 24-35. Contemplated variants of any one of SEQ ID NO: 24-35 include but are not limited to polypeptide sequences having at least from about 60.1% to about 65% identity to that of any one of SEQ ID NO: 24-35. Contemplated variants of any one of SEQ ID NO: 24-35 include but are not limited to polypeptide sequences having at least from about 65.1% to about 70% identity to that of any one of SEQ ID NO: 24-35. Contemplated variants of any one of SEQ ID NO: 24-35 include but are not limited to polypeptide having at least from about 70.1% to about 75% identity to that of any one of SEQ ID NO: 24-35. Contemplated variants of any one of SEQ ID NO: 24-35 include but are not limited to polypeptide sequences having at least from about 75.1% to about 80% identity to that of any one of SEQ ID NO: 24-35. Contemplated variants of any one of SEQ ID NO: 24-35 include but are not limited to polypeptide sequences having at least from about 80.1% to about 85% identity to that of any one of SEQ ID NO: 24-35. Contemplated variants of any one of SEQ ID NO: 24-35 include but are not limited to polypeptide sequences having at least from about 85.1% to about 90% identity to that of any one of SEQ ID NO: 24-35. Contemplated variant of any one of SEQ ID NO: 24-35 include but are not limited to polypeptide sequences having at least from about 90.1% to about 95% identity to that of any one of SEQ ID NO: 24-35. Contemplated variants of any one of SEQ ID NO: 24-35 include but are not limited to polypeptide sequences having at least from about 95.1% to about 97% identity to that of any one of SEQ ID NO: 24-35. Contemplated variant of any one of SEQ ID NO: 24-35 include but are not limited to polypeptide sequences having at least from about 97.1% to about 99% identity to that of any one of SEQ ID NO: 24-35.

The invention is directed to a polypeptide sequence comprising from about 10 to about 50 consecutive amino acids from any one of SEQ ID NO: 24-35. The invention is directed to a polypeptide sequence comprising from about 10 to about 100 consecutive amino acids from any one of SEQ ID NO: 24-35. The invention is directed to a polypeptide sequence comprising from about 10 to about 150 consecutive amino acids from any one of SEQ ID NO: 24-35. The invention is directed to a polypeptide sequence comprising from about 10 to about 200 consecutive amino acids from any one of SEQ ID NO: 24-35. The invention is directed to a polypeptide sequence comprising from about 10 to about 250 consecutive amino acids from any one of SEQ ID NO: 24-35. The invention is directed to a polypeptide sequence comprising from about 10 to about 300 consecutive amino acids from any one of SEQ ID NO: 24-35. The invention is directed to a polypeptide sequence comprising from about 10 to about 350 consecutive amino acids from any one of SEQ ID NO: 24-35. The invention is directed to a polypeptide sequence comprising from about 10 to about 400 consecutive amino acids from any one of SEQ ID NO: 24-35. The invention is directed to a polypeptide sequence comprising from about 10 to about 450 consecutive amino acids from any one of SEQ ID NO: 24-35. The invention is directed to a polypeptide sequence comprising from about 10 to about 460 consecutive amino acids from any one of SEQ ID NO: 24-35. The invention is directed to a polypeptide sequence comprising from about 10 to about 470 consecutive amino acids from any one of SEQ ID NO: 24-35. The invention is directed to a polypeptide sequence comprising from about 10 to about 480 consecutive amino acids from any one of SEQ ID NO: 24-35. The invention is directed to a polypeptide sequence comprising from about 10 to about 490 consecutive amino acids from any one of SEQ ID NO: 24-35. The invention is further directed to polypeptide sequences having from about 50% to about 99% identity to a polypeptide sequence comprising from about 10 to about 490 consecutive amino acids from any one of SEQ ID NO: 24-35. The invention is further directed to polypeptide sequences having from about 50% to about 99% identity to a polypeptide sequence comprising from about 10 to about 550 consecutive amino acids from any one of SEQ ID NO: 24-35. The invention is further directed to polypeptide sequences having from about 50% to about 99% identity to a polypeptide sequence comprising from about 10 to about 600 consecutive amino acids from any one of SEQ ID NO: 24-35. The invention is further directed to polypeptide sequences having from about 50% to about 99% identity to a polypeptide sequence comprising from about 10 to about 650 consecutive amino acids from any one of SEQ ID NO: 24-35. The invention is further directed to polypeptide sequences having from about 50% to about 99% identity to a polypeptide sequence comprising from about 10 to about 685 consecutive amino acids from any one of SEQ ID NO: 24-35. In certain embodiments, the invention is directed to isolated and purified peptides.

In certain embodiments, the polypeptides of the present invention can be suitable for use as antigens to detect antibodies against picornaviruses represented by SEQ ID NOs: 1-23, and variants thereof. In other embodiments, the polypeptides of the present invention which comprise antigenic determinants can be used in various immunoassays to identify subjects exposed to and/or samples which comprise picornaviruses represented by SEQ ID NO: 1-23, and variants thereof.

In another aspect, the invention is directed to an antibody which specifically binds to amino acids from the polypeptide of any one of SEQ ID NO: 24-35. In one embodiment the antibody is purified. The antibodies can be polyclonal or monoclonal. The antibodies can also be chimeric (i.e., a combination of sequences from more than one species, for example, a chimeric mouse-human immunoglobulin), humanized or fully-human. Human antibodies avoid certain of the problems associated with antibodies that possess murine or rat (or other species) variable and/or constant regions. The presence of such murine or rat derived proteins can lead to the rapid clearance of the antibodies or can lead to the generation of an immune response against the antibody by a patient. In order to avoid the utilization of murine or rat derived antibodies, one can develop humanized antibodies or generate fully human antibodies through the introduction of human antibody function into a rodent so that the rodent would produce antibodies having fully human sequences. For example, U.S. Pat. Nos. 5,770,429; 6,150,584; and 6,677,138 relate to transgenic mouse technology, i.e., the HuMAb-Mouse™ or the Xenmouse®, to produce high affinity, fully human antibodies to a target antigen.

Immunogenic sequences are contained in the capsid proteins VP4, VP2, VP3, and VP1, with VP1 being the important one for receptor interaction. In order to raise protective antibodies (vaccine) one may use VP1. In another embodiment, one can use the whole P1 region (comprised of VP4/VP2/VP3/VP1). The P1 region extends in the full length sequence from nt 599 to 3119 (aa 1-840) with VP4 from nt 599-799, aa 1-67; VP2 nt 800-1585, aa 68-329; VP3 nt 1586-2284, aa 330-562; VP1 nt 2285-3119, aa 563-840 (see map below at end of examples section).

Antibodies can bind to other molecules (antigens) via heavy and light chain variable domains, V.sub.H and V.sub.L, respectively. Antibodies include IgG, IgD, IgA, IgM and IgE. The antibodies may be intact immunoglobulin molecules, two full length heavy chains linked by disulfide bonds to two full length light chains, as well as subsequences (i.e. fragments) of immunoglobulin molecules that bind to an epitope of an antigen, or subsequences thereof (i.e. fragments) of immunoglobulin molecules, with or without constant region, that bind to an epitope of an antigen. Antibodies may comprise full length heavy and light chain variable domains, V.sub.H and V.sub.L, individually or in any combination.

The basic immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V.sub.1) and variable heavy chain (V.sub.H) refer to these light and heavy chains respectively.

Antibodies may exist as intact immunoglobulins or as a number of well characterized fragments produced by digestion with various peptidases. In particular, pepsin digests an antibody below the disulfide linkages in the hinge region to produce F(ab)′.sub.2, a dimer of Fab which itself is a light chain joined to V.sub.H-C.sub.H1 by a disulfide bond. The F(ab)′.sub.2 may be reduced under mild conditions to break the disulfide linkage in the hinge region thereby converting the F(ab)′.sub.2 dimer into an Fab′ monomer. The Fab′ monomer is essentially an Fab with part of the hinge region (see, Fundamental Immunology, W. E. Paul, ed., Raven Press, N.Y. (1993) for more antibody fragment terminology). While the Fab′ domain is defined in terms of the digestion of an intact antibody, one of skill will appreciate that such Fab′ fragments may be synthesized de novo either chemically or by utilizing recombinant DNA methodology.

The Fab′ regions may be derived from antibodies of animal (especially mouse or rat) or human origin or may be chimeric (Morrison et al., Proc Natl. Acad. Sci. USA 81, 6851-6855 (1984) both incorporated by reference herein) or humanized (Jones et al., Nature 321, 522-525 (1986), and published UK patent application No. 8707252, both incorporated by reference herein).

An antibody described in this application can include or be derived from any mammal, such as but not limited to, a human, a mouse, a rabbit, a rat, a rodent, a primate, or any combination thereof and includes isolated human, primate, rodent, mammalian, chimeric, humanized and/or CDR-grafted or CDR-adapted antibodies, immunoglobulins, cleavage products and other portions and variants thereof.

Antibodies useful in the embodiments of the invention can be derived in several ways well known in the art. In one aspect, the antibodies can be obtained using any of the hybridoma techniques well known in the art, see, e.g., Ausubel, et al., ed., Current Protocols in Molecular Biology, John Wiley & Sons, Inc., NY, N.Y. (1987-2001); Sambrook, et al., Molecular Cloning: A Laboratory Manual, 2.sup.nd Edition, Cold Spring Harbor, N.Y. (1989); Harlow and Lane, Antibodies, A Laboratory Manual, Cold Spring Harbor, N.Y. (1989); Colligan, et al., eds., Current Protocols in Immunology, John Wiley & Sons, Inc., NY (1994-2001); Colligan et al., Current Protocols in Protein Science, John Wiley & Sons, NY, N.Y., (1997-2001).

The antibodies may also be obtained from selecting from libraries of such domains or components, e.g. a phage library. A phage library can be created by inserting a library of random oligonucleotides or a library of polynucleotides containing sequences of interest, such as from the B-cells of an immunized animal or human (Smith, G. P. 1985. Science 228: 1315-1317). Antibody phage libraries contain heavy (H) and light (L) chain variable region pairs in one phage allowing the expression of single-chain Fv fragments or Fab fragments (Hoogenboom, et al. 2000, Immunol Today 21(8) 371-8). The diversity of a phagemid library can be manipulated to increase and/or alter the immunospecificities of the monoclonal antibodies of the library to produce and subsequently identify additional, desirable, human monoclonal antibodies. For example, the heavy (H) chain and light (L) chain immunoglobulin molecule encoding genes can be randomly mixed (shuffled) to create new HL pairs in an assembled immunoglobulin molecule. Additionally, either or both the H and L chain encoding genes can be mutagenized in a complementarity determining region (CDR) of the variable region of the immunoglobulin polypeptide, and subsequently screened for desirable affinity and neutralization capabilities. Antibody libraries also can be created synthetically by selecting one or more human framework sequences and introducing collections of CDR cassettes derived from human antibody repertoires or through designed variation (Kretzschmar and von Ruden 2000, Current Opinion in Biotechnology, 13:598-602). The positions of diversity are not limited to CDRs but can also include the framework segments of the variable regions or may include other than antibody variable regions, such as peptides.

Other target binding components which may include other than antibody variable regions are ribosome display, yeast display, and bacterial displays. Ribosome display is a method of translating mRNAs into their cognate proteins while keeping the protein attached to the RNA. The nucleic acid coding sequence is recovered by RT-PCR (Mattheakis, L. C. et al. 1994. Proc Natl Acad Sci USA 91, 9022). Yeast display is based on the construction of fusion proteins of the membrane-associated alpha-agglutinin yeast adhesion receptor, aga1 and aga2, a part of the mating type system (Broder, et al. 1997. Nature Biotechnology, 15:553-7). Bacterial display is based fusion of the target to exported bacterial proteins that associate with the cell membrane or cell wall (Chen and Georgiou 2002. Biotechnol Bioeng, 79:496-503).

In comparison to hybridoma technology, phage and other antibody display methods afford the opportunity to manipulate selection against the antigen target in vitro and without the limitation of the possibility of host effects on the antigen or vice versa.

Specific examples of antibody subsequences include, for example, Fab, Fab′, (Fab′).sub.2, Fv, or single chain antibody (SCA) fragment (e.g., scFv). Subsequences include portions which retain at least part of the function or activity of full length sequence. For example, an antibody subsequence will retain the ability to selectively bind to an antigen even though the binding affinity of the subsequence may be greater or less than the binding affinity of the full length antibody.

Pepsin or papain digestion of whole antibodies can be used to generate antibody fragments. In particular, an Fab fragment consists of a monovalent antigen-binding fragment of an antibody molecule, and can be produced by digestion of a whole antibody molecule with the enzyme papain, to yield a fragment consisting of an intact light chain and a portion of a heavy chain. An (Fab′).sub.2 fragment of an antibody can be obtained by treating a whole antibody molecule with the enzyme pepsin, without subsequent reduction. An Fab′ fragment of an antibody molecule can be obtained from (Fab′).sub.2 by reduction with a thiol reducing agent, which yields a molecule consisting of an intact light chain and a portion of a heavy chain. Two Fab′ fragments are obtained per antibody molecule treated in this manner.

An Fv fragment is a fragment containing the variable region of a light chain V.sub.L and the variable region of a heavy chain V.sub.H expressed as two chains. The association may be non-covalent or may be covalent, such as a chemical cross-linking agent or an intermolecular disulfide bond (Inbar et al., (1972) Proc. Natl. Acad. Sci. USA 69:2659; Sandhu (1992) Crit. Rev. Biotech. 12:437).

A single chain antibody (“SCA”) is a genetically engineered or enzymatically digested antibody containing the variable region of a light chain V.sub.L and the variable region of a heavy chain, optionally linked by a flexible linker, such as a polypeptide sequence, in either V.sub.L-linker-V.sub.H orientation or in V.sub.H-linker-V.sub.L orientation. Alternatively, a single chain Fv fragment can be produced by linking two variable domains via a disulfide linkage between two cysteine residues. Methods for producing scFv antibodies are described, for example, by Whitlow et al., (1991) In: Methods: A Companion to Methods in Enzymology 2:97; U.S. Pat. No. 4,946,778; and Pack et al., (1993) Bio/Technology 11:1271.

Other methods of producing antibody subsequences, such as separation of heavy chains to form monovalent light-heavy chain fragments, further cleavage of fragments, or other enzymatic, chemical, or genetic techniques may also be used, provided that the subsequences bind to the antigen to which the intact antibody binds.

Antibodies used in the invention, include full length antibodies, subsequences (e.g., single chain forms), dimers, trimers, tetramers, pentamers, hexamers or any other higher order oligomer that retains at least a part of antigen binding activity of monomer. Multimers can comprise heteromeric or homomeric combinations of full length antibody, subsequences, unmodified or modified as set forth herein and known in the art. Antibody multimers are useful for increasing antigen avidity in comparison to monomer due to the multimer having multiple antigen binding sites. Antibody multimers are also useful for producing oligomeric (e.g., dimer, trimer, tertamer, etc.) combinations of different antibodies thereby producing compositions of antibodies that are multifunctional (e.g., bifunctional, trifunctional, tetrafunctional, etc.).

Antibodies can be produced through chemical crosslinking of the selected molecules (which have been produced by synthetic means or by expression of nucleic acid that encode the polypeptides) or through recombinant DNA technology combined with in vitro, or cellular expression of the polypeptide, and subsequent oligomerization. Antibodies can be similarly produced through recombinant technology and expression, fusion of hybridomas that produce antibodies with different epitopic specificities, or expression of multiple nucleic acid encoding antibody variable chains with different epitopic specificities in a single cell.

Antibodies may be either joined directly or indirectly through covalent or non-covalent binding, e.g. via a multimerization domain, to produce multimers. A “multimerization domain” mediates non-covalent protein-protein interactions. Specific examples include coiled-coil (e.g., leucine zipper structures) and alpha-helical protein sequences. Sequences that mediate protein-protein binding via Van der Waals' forces, hydrogen bonding or charge-charge bonds are also contemplated as multimerization domains. Additional examples include basic-helix-loop-helix domains and other protein sequences that mediate heteromeric or homomeric protein-protein interactions among nucleic acid binding proteins (e.g., DNA binding transcription factors, such as TAFs). One specific example of a multimerization domain is p53 residues 319 to 360 which mediate tetramer formation. Another example is human platelet factor 4, which self-assembles into tetramers. Yet another example is extracellular protein TSP4, a member of the thrombospondin family, which can form pentamers. Additional specific examples are the leucine zippers of jun, fos, and yeast protein GCN4.

Antibodies may be directly linked to each other via a chemical cross linking agent or can be connected via a linker sequence (e.g., a peptide sequence) to form multimers.

The antibodies of the present invention can be used to modulate the activity of the polypeptide of any one of SEQ ID NO: 24-35, variants or fragments thereof. In certain aspects, the invention is directed to a method for treating a subject, the method comprising administering to the subject an antibody which specifically binds to amino acids from the polypeptide of any one of SEQ ID NO: 24-35. In certain embodiments, antibody binding to the polypeptide of any one of SEQ ID NO: 24-35 may interfere or inhibit the function of the polypeptide, thus providing a method to inhibit virus propagation and spreading. In certain embodiments, the polypeptide is VP1. In other embodiments, the polypeptide is VP4. Thus the invention provides a method for treating a subject suffering from ILI.

In other embodiments, the antibodies of the invention can be used to purify polypeptides of any one of SEQ ID NO: 24-35, variants or fragments thereof. In other embodiments, the antibodies of the invention can be used to identify expression and localization of the polypeptide of any one of SEQ ID NO: 24-35, variants, fragments or domains thereof. Analysis of expression and localization of the polypeptide of any one of SEQ ID NO: 24-35 can be useful in determining potential role of the polypeptide of any one of SEQ ID NO: 24-35 in the ethiology and progression of diseases, syndromes and disorders dependent on cellular regulation of iron levels.

In other embodiments, the antibodies of the present invention can be used in various immunoassays to identify subjects exposed to and/or samples which comprise antigens from picornaviruses represented by SEQ ID NOs: 1-23, and variants thereof.

Any suitable immunoassay which can lead to formation of antigen-antibody complex is contemplated by the present invention. Variations and different formats of immunoassays, for example but not limited to ELISA, lateral flow assays for detection of analytes in samples, immunoprecipitation, are known in the art, and are contemplated by the invention. In various embodiments, the antigen and/or the antibody can be labeled by any suitable label or method known in the art, for example but not limited to enzymatic, Immunoassays may use solid supports, or immunoprecipitation. Immunoassays which amplify the signal from the antigen-antibody immune complex are also contemplated.

In certain aspects the invention provides methods for assaying a sample to determine the presence or absence of a picornaviruses comprising SEQ ID NOs: 1-23, as provided by the invention, and variants thereof. The invention contemplates various methods for assaying a sample, including, but not limited to, methods which can detect the presence of nucleic acids, methods which can detect the presence of antigens, methods which can detect the presence of antibodies against antigens from polypeptides encoded by SEQ ID NO: 1-23, or polypeptides of SEQ ID NO: 24-35, as provided by the invention, and variants thereof.

Immunogenic Compositions

In certain aspects, the present invention provides immunogenic compositions capable of inducing an immune response against picornaviruses including the rhinoviruses of the invention comprising any one of SEQ ID NO: 1-23. In one embodiment, the immunogenic compositions are capable of ameliorating the symptoms of a picornaviral infection and/or of reducing the duration of a picornaviral infection. In another embodiment, the immunogenic compositions are capable of inducing protective immunity against picornaviral infection. The immunogenic compositions of the invention can be effective against the picornaviruses disclosed herein, and may also be cross-reactive with, and effective against, multiple different clades and strains of rhinoviruses, and against other picornaviruses.

The types of immunogenic composition encompassed by the invention include, but are not limited to, attenuated live viral vaccines, inactivated (killed) viral vaccines, and subunit vaccines.

The rhinoviruses of the invention may be attenuated by removal or disruption of those viral sequences whose products cause or contribute to the disease and symptoms associated with rhinoviral infection, and leaving intact those sequences required for viral replication. In this way an attenuated rhinovirus can be produced that replicates in subjects, and induces an immune response in subjects, but which does not induce the deleterious disease and symptoms usually associated with rhinoviral infection. One of skill in the art can determine which rhinoviral sequences can or should be removed or disrupted, and which sequences should be left intact, in order to generate an attenuated rhinovirus suitable for use as a vaccine.

The novel rhinoviruses of the invention may be also be inactivated, such as by chemical treatment, to “kill” the viruses such that they are no longer capable of replicating or causing disease in subjects, but still induce an immune response in a subject. There are many suitable viral inactivation methods known in the art and one of skill in the art can readily select a suitable method and produce an inactivated “killed” rhinovirus suitable for use as a vaccine.

The immunogenic compositions of the invention may comprise subunit vaccines. Subunit vaccines include nucleic acid vaccines such as DNA vaccines, which contain nucleic acids that encode one or more viral proteins or subunits, or portions of those proteins or subunits. When using such vaccines, the nucleic acid is administered to the subject, and the immunogenic proteins or peptides encoded by the nucleic acid are expressed in the subject, such that an immune response against the proteins or peptides is generated in the subject. Subunit vaccines may also be proteinaceous vaccines, which contain the viral proteins or subunits themselves, or portions of those proteins or subunits.

To make the nucleic acid and DNA vaccines of the invention the rhinoviral sequences disclosed herein may be incorporated into a plasmid or expression vector containing the nucleic acid that encodes the viral protein or peptide. Any suitable plasmid or expression vector capable of driving expression of the protein or peptide in the subject may be used. Such plasmids and expression vectors should include a suitable promoter for directing transcription of the nucleic acid. The nucleic acid sequence(s) that encodes the rhinoviral protein or peptide may also be incorporated into a suitable recombinant virus for administration to the subject. Examples of suitable viruses include, but are not limited to, vaccinia viruses, retroviruses, adenoviruses and adeno-associated viruses. One of skill in the art could readily select a suitable plasmid, expression vector, or recombinant virus for delivery of the rhinoviral nucleic acid sequences of the invention.

To produce the proteinaceous vaccines of the invention, the rhinoviral nucleic acid sequences of the invention are delivered to cultured cells, for example by transfecting cultured cells with plasmids or expression vectors containing the rhinoviral nucleic acid sequences, or by infecting cultured cells with recombinant viruses containing the rhinoviral nucleic acid sequences. The rhinoviral proteins or peptides may then be expressed in the cultured cells and purified. The purified proteins can then be incorporated into compositions suitable for administration to subjects. Methods and techniques for expression and purification of recombinant proteins are well known in the art, and any such suitable methods may be used.

Subunit vaccines of the present invention may encode or contain any of the rhinoviral proteins or peptides described herein, or any portions, fragments, derivatives or mutants thereof, that are immunogenic in a subject. One of skill in the art can readily test the immunogenicity of the rhinoviral proteins and peptides described herein, and can select suitable proteins or peptides to use in subunit vaccines.

The immunogenic compositions of the invention comprise at least one rhinovirus-derived immunogenic component, such as those described above. The compositions may also comprise one or more additives including, but not limited to, one or more pharmaceutically acceptable carriers, buffers, stabilizers, diluents, preservatives, solubilizers, liposomes or immunomodulatory agents. Suitable immunomodulatory agents include, but are not limited to, adjuvants, cytokines, polynucleotide encoding cytokines, and agents that facilitate cellular uptake of the rhinovirus-derived immunogenic component.

Immunogenic compositions for use in accordance with the present invention thus may be formulated in a conventional manner using one or more physiologically acceptable carriers comprising excipients and auxiliaries which facilitate processing of the active compounds into preparations which can be used to induce an immunogenic response. These immunogenic compositions may be manufactured in a manner that is itself known, e.g. by means of conventional mixing, dissolving, granulating, dragee-making, levigating, emulsifying, encapsulating, entrapping or lyophilizing processes. Proper formulation is dependent upon the route of administration chosen. When a therapeutically effective amount of protein or other active ingredient of the present invention is administered orally, protein or other active ingredient of the present invention can be in the form of a tablet, capsule, powder, solution or elixr. When administered in tablet form, the immunogenic composition of the invention may additionally contain a solid carrier such as a gelatin or an adjuvant. The tablet, capsule, and powder contain from about 5 to 95% protein or other active ingredient of the present invention, and from about 25 to 90% protein or other active ingredient of the present invention. When administered in liquid form, a liquid carrier such as water, petroleum, oils of animal or plant origin such as peanut oil, mineral oil, soybean oil, or sesame oil, or synthetic oils may be added. The liquid form of the immunogenic composition may further contain physiological saline solution, dextrose or other saccharide solution, or glycols such as ethylene glycol, propylene glycol or polyethylene glycol. When administered in liquid form, the immunogenic composition contains from about 0.5 to 90% by weight of protein or other active ingredient of the present invention, and from about 1 to 50% protein or other active ingredient of the present invention.

When a therapeutically effective amount of protein or other active ingredient of the present invention is administered by intravenous, cutaneous or subcutaneous injection, protein or other active ingredient of the present invention will be in the form of a pyrogen-free, parenterally acceptable aqueous solution. The preparation of such parenterally acceptable protein or other active ingredient solutions, having due regard to pH, isotonicity, stability, and the like, is within the skill in the art. One immunogenic composition for intravenous, cutaneous, or subcutaneous injection can contain, in addition to protein or other active ingredient of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The immunogenic composition of the present invention may also contain stabilizers, preservatives, buffers, antioxidants, or other additives known to those of skill in the art. For injection, the agents of the invention may be formulated in aqueous solutions, physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with immunogenically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Immunogenic preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Immunogenic preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch. The compounds may be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents.

Immunogenic formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient maybe in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides. In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A carrier for hydrophobic compounds of the invention can be a co-solvent system comprising benzyl alcohol, a nonpolar surfactant, a water-miscible organic polymer, and an aqueous phase. The co-solvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may substitute for dextrose. Alternatively, other delivery systems for hydrophobic immunogenic compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various types of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein or other active ingredient stabilization may be employed.

The immunogenic compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols. Many of the active ingredients of the invention may be provided as salts with immunogenically compatible counter ions. Such immunogenically acceptable base addition salts are those salts which retain the biological effectiveness and properties of the free acids and which are obtained by reaction with inorganic or organic bases such as sodium hydroxide, magnesium hydroxide, ammonia, trialkylamine, dialkylamine, monoalkylamine, dibasic amino acids, sodium acetate, potassium benzoate, triethanol amine and the like.

The immunogenic composition of the invention may be in the form of a complex of the protein(s) or other active ingredient of present invention along with protein or peptide antigens. The protein and/or peptide antigen will deliver a stimulatory signal to both B and T lymphocytes. B lymphocytes will respond to antigen through their surface immunoglobulin receptor. T lymphocytes will respond to antigen through the T cell receptor (TCR) following presentation of the antigen by MHC proteins. MHC and structurally related proteins including those encoded by class I and class II MHC genes on host cells will serve to present the peptide antigen(s) to T lymphocytes. The antigen components could also be supplied as purified MHC-peptide complexes alone or with co-stimulatory molecules that can directly signal T cells. Alternatively antibodies able to bind surface immunoglobulin and other molecules on B cells as well as antibodies able to bind the TCR and other molecules on T cells can be combined with the immunogenic composition of the invention.

The immunogenic composition of the invention may be in the form of a liposome in which protein of the present invention is combined, in addition to other acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithins, phospholipids, saponin, bile acids, and the like. Preparation of such liposomal formulations is within the level of skill in the art, as disclosed, for example, in U.S. Pat. Nos. 4,235,871; 4,501,728; 4,837,028; and 4,737,323, all of which are incorporated herein by reference.

Other additives that are useful in vaccine formulations are known and will be apparent to those of skill in the art.

An “immunologically effective amount” of the compositions of the invention may be administered to a subject. As used herein, the term “immunologically effective amount” refers to an amount capable of inducing, or enhancing the induction of, the desired immune response in a subject. The desired response may include, inter alia, inducing an antibody or cell-mediated immune response, or both. The desired response may also be induction of an immune response sufficient to ameliorate the symptoms of a rhinoviral infection, reduce the duration of a rhinoviral infection, and/or provide protective immunity in a subject against subsequent challenge with a rhinovirus. An immunologically effective amount may be an amount that induces actual “protection” against rhinoviral infection, meaning the prevention of any of the symptoms or conditions resulting from rhinoviral infection in subjects. An immunologically effective amount may also be an amount sufficient to delay the onset of symptoms and conditions associated with infection, reduce the degree or rate of infection, reduce in the severity of any disease or symptom resulting from infection, and reduce the viral load of an infected subject.

One of skill in the art can readily determine what is an “immunologically effective amount” of the compositions of the invention without performing any undue experimentation. An effective amount can be determined by conventional means, starting with a low dose of and then increasing the dosage while monitoring the immunological effects. Numerous factors can be taken into consideration when determining an optimal amount to administer, including the size, age, and general condition of the subject, the presence of other drugs in the subject, the virulence of the particular rhinovirus against which the subject is being vaccinated, and the like. The actual dosage is can be chosen after consideration of the results from various animal studies.

The immunologically effective amount of the immunogenic composition may be administered in a single dose, in divided doses, or using a “prime-boost” regimen. The compositions may be administered by any suitable route, including, but not limited to parenteral, intradermal, transdermal, subcutaneous, intramuscular, intravenous, intraperitoneal, intranasal, oral, or intraocular routes, or by a combination of routes. The compositions may also be administered using a “gun” device which fires particles, such as gold particles, onto which compositions of the present invention have been coated, into the skin of a subject. The skilled artisan will be able to formulate the vaccine composition according to the route chosen.

Viral Purification

Methods of purification of inactivated virus are known in the art and may include one or more of, for instance gradient centrifugation, ultracentrifugation, continuous-flow ultracentrifugation and chromatography, such as ion exchange chromatography, size exclusion chromatography, and liquid affinity chromatography. Additional method of purification include ultrafiltration and dialfiltration. See J P Gregersen “Herstellung von Virussimpfstoffen aus Zellkulturen” Chapter 4.2 in Pharmazeutische Biotechnology (eds. O. Kayser and R H Mueller) Wissenschaftliche Verlagsgesellschaft, Stuttgart, 2000. See also, O'Neil et al., “Virus Harvesting and Affinity Based Liquid Chromatography. A Method for Virus Concentration and Purification”, Biotechnology (1993) 11:173-177; Prior et al., “Process Development for Manufacture of Inactivated HIV-1”, Pharmaceutical Technology (1995) 30-52; and Majhdi et al., “Isolation and Characterization of a Coronavirus from Elk Calves with diarrhea” Journal of Clinical Microbiology (1995) 35(11): 2937-2942.

Other examples of purification methods suitable for use in the invention include polyethylene glycol or ammonium sulfate precipitation (see Trepanier et al., “Concentration of human respiratory syncytial virus using ammonium sulfate, polyethylene glycol or hollow fiber ultrafiltration” Journal of Virological Methods (1981) 3(4):201-211; Hagen et al., “Optimization of Poly(ethylene glycol) Precipitation of Hepatitis Virus Used to prepare VAQTA, a Highly Purified Inactivated Vaccine” Biotechnology Progress (1996) 12:406-412; and Carlsson et al., “Purification of Infectious Pancreatic Necrosis Virus by Anion Exchange Chromatography Increases the Specific Infectivity” Journal of Virological Methods (1994) 47:27-36) as well as ultrafiltration and microfiltration (see Pay et al., Developments in Biological Standardization (1985) 60:171-174; Tsurumi et al., “Structure and filtration performances of improved cuprammonium regenerated cellulose hollow fibre (improved BMM hollow fibre) for virus removal” Polymer Journal (1990) 22(12):1085-1100; and Makino et al., “Concentration of live retrovirus with a regenerated cellulose hollow fibre, BMM”, Archives of Virology (1994) 139(1-2):87-96.).

Viruses can be purified using chromatography, such as ion exchange, chromatography. Chromatic purification allows for the production of large volumes of virus containing suspension. The viral product of interest can interact with the chromatic medium by a simple adsorption/desorption mechanism, and large volumes of sample can be processed in a single load. Contaminants which do not have affinity for the adsorbent pass through the column. The virus material can then be eluted in concentrated form.

Anion exchange resins that may be used include DEAE, EMD TMAE. Cation exchange resins may comprise a sulfonic acid-modified surface. Viruses can be purified using ion exchange chromatography comprising a strong anion exchange resin (e.g. EMD TMAE) for the first step and EMD-SO.sub.3 (cation exchange resin) for the second step. A metal-binding affinity chromatography step can optionally be included for further purification. (See, e.g., WO 97/06243).

A resin such as Fractogel™ EMD. Can also be used This synthetic methacrylate based resin has long, linear polymer chains (so-called “tentacles”) covalently attached. This “tentacle chemistry” allows for a large amount of sterically accessible ligands for the binding of biomolecules without any steric hindrance. This resin also has improved pressure stability.

Column-based liquid affinity chromatography is another purification method that can be used invention. One example of a resin for use in purification method is Matrex™ Cellufine™ Sulfate (MCS). MCS consists of a rigid spherical (approx. 45-105.mu.m diameter) cellulose matrix of 3,000 Dalton exclusion limit (its pore structure excludes macromolecules), with a low concentration of sulfate ester functionality on the 6-position of cellulose. As the functional ligand (sulfate ester) is relatively highly dispersed, it presents insufficient cationic charge density to allow for most soluble proteins to adsorb onto the bead surface. Therefore the bulk of the protein found in typical virus pools (cell culture supernatants, e.g. pyrogens and most contaminating proteins, as well as nucleic acids and endotoxins) are washed from the column and a degree of purification of the bound virus is achieved.

The rigid, high-strength beads of MCS tend to resist compression. The pressure/flow characteristics the MCS resin permit high linear flow rates allowing high-speed processing, even in large columns, making it an easily scalable unit operation. In addition a chromatographic purification step with MCS provides increased assurance of safety and product sterility, avoiding excessive product handling and safety concerns. As endotoxins do not bind to it, the MCS purification step allows a rapid and contaminant free depyrogenation. Gentle binding and elution conditions provide high capacity and product yield. The MCS resin therefore represents a simple, rapid, effective, and cost-saving means for concentration, purification and depyrogenation. In addition, MCS resins can be reused repeatedly.

Inactivated viruses may be further purified by gradient centrifugation, or density gradient centrifugation. For commercial scale operation a continuous flow sucrose gradient centrifugation would be an option. This method is widely used to purify antiviral vaccines and is known to one skilled in the art (See J P Gregersen “Herstellung von Virussimpfstoffen aus Zellkulturen” Chapter 4.2 in Pharmazeutische Biotechnology (eds. O. Kayser and R H Mueller) Wissenschaftliche Verlagsgesellschaft, Stuttgart, 2000.)

Additional purification methods which may be used to purify viruses of the invention include the use of a nucleic acid degrading agent, a nucleic acid degrading enzyme, such as a nuclease having DNase and RNase activity, or an endonuclease, such as from Serratia marcescens, commercially available as Benzonase™, membrane adsorbers with anionic functional groups (e.g. Sartobind™) or additional chromatographic steps with anionic functional groups (e.g. DEAE or TMAE). An ultrafiltration/dialfiltration and final sterile filtration step could also be added to the purification method.

The purified viral preparation of the invention is substantially free of contaminating proteins derived from the cells or cell culture and can comprises less than about 1000, 500, 250, 150, 100, or 50 pg cellular nucleic acid/.mu.g virus antigen, and less than about 1000, 500, 250, 150, 100, or 50 pg cellular nucleic acid/dose. The purified viral preparation can also comprises less than about 20 pg or less than about 10 pg. Methods of measuring host cell nucleic acid levels in a viral sample are known in the art. Standardized methods approved or recommended by regulatory authorities such as the WHO or the FDA can be used.

It will be readily apparent to those skilled in the relevant arts that other suitable modifications and adaptations to the methods and applications described herein can be made without departing from the scope of the invention or any embodiment thereof.

The following examples illustrate the invention described herein, and are set forth to aid in the understanding of the invention, and should not be construed to limit in any way the scope of the invention as defined in the claims which follow thereafter.

The following methods can be used in connection with the embodiments of the invention.

### EXAMPLES

### Example 1

### Detection of Pathogens in Respiratory Specimens

Study Population and Clinical Materials

Patients with ILI were identified during visits to New York State health care providers belonging to the CDC Influenza Sentinel Physicians Network during the 2004-2005 influenza season (October 1-May 31). Respiratory swabs were obtained in M4 medium (Remel, Lenexa, Kans.) and transported to the Clinical Virology Program of the New York State Department of Health's Wadsworth Center Laboratory. The patients in this study ranged from 4 months to 98 years of age, with a median age of 25 years.

Initial Laboratory Testing

Penicillin, streptomycin, and mycostatin were added to specimens prior to inoculation into primary rhesus monkey kidney (RhMK) cells for conventional virus culture. In addition, samples were tested for the presence of FLUAV or FLUBV, using direct antigen detection (BD Directigen, Franklin Lakes, N.J.) or real-time RT-PCR assays. The primer and probe sequences utilized were those provided by the CDC to the Laboratory Response Network. Assays were optimized and validated at the Wadsworth Center. RNA was extracted for initial real-time RT-PCR from 140 μl of specimen using Qiagen viral RNA kits and was eluted into a 60-μl volume (Qiagen, Valencia, Calif.). Inoculated RhMK cells were observed three times per week for 2 weeks for cytopathic effect (CPE). Between days 7 and 10, cultures were tested for hemadsorption (HAD), and positive cultures were assayed for influenza and parainfluenza viruses, by direct immunofluorescence assay (IFA; Chemicon, Temecula, Calif.). When CPE was observed in the absence of HAD, cells were tested by IFA for adenovirus (Chemicon) and herpes simplex virus (HSV-1, -2; MicroTrack, San Jose, Calif.).

MassTag PCR

Total nucleic acids were extracted from 250 μl of specimen, which had been stored at −80° C., using the NucliSens® Magnetic Extraction Method on the miniMAG platform, and was eluted into a 50-μl volume (bioMérieux, Durham, N.C.). Prior to extraction, samples were spiked with a quality-control transcript encoding a portion of the green fluorescent protein (GFP), which was subsequently amplified in a concurrent RT-PCR assay to control for extraction efficiency and the absence of inhibitors in the RT or PCR reaction. The MassTag PCR assay targeted the following respiratory pathogens: FLUAV; FLUBV; human respiratory syncytial viruses A (HRVS-A) and B (HRSV-B); human coronaviruses OC43, 229E, and SARS(HCoV-OC43, HCoV-229E, HCoV-SARS); human parainfluenza viruses 1 through 3 (HPIV-1, -2, -3); human metapneumovirus (HMPV); human enterovirus (HEV); human adenovirus (HADV); M. pneumoniae; L. pneumophila; C. pneumoniae; S. pneumoniae; H. influenzae; and N. meningitidis. A primer pair targeting the GFP quality-control sequence was also included. MassTag PCR amplification products were analyzed in a single quadrapole mass spectrometer using positive-mode atmospheric pressure chemical ionization.

Additional Molecular Analyses

Samples testing positive in the MassTag PCR for M. pneumoniae, L. pneumophila, C. pneumoniae, N. meningitidis, HEV, and HADV were also tested for these targets using diagnostic real-time PCR assays approved for clinical use through the New York State Department of Health Clinical Laboratory Evaluation Program (CLEP). Published PCR assays were applied for samples positive for HCoV-OC43, HCoV-229E (van Elden et al., 2004), H. influenzae (types b and c (Corless et al., 2001)), and S. pneumoniae (serotypes 1, 3, 5, 6, 7, 9, 14, 19, 22, 23, 29, and 46 (Hassen-King et al., 1994)). The VP4 and VP1 gene regions of picornaviruses were amplified as described (Coiras et al., 2004; Nix et al., in press). MassTag PCR amplification products, other than those for influenza viruses, were cloned into pGEM-Teasy plasmid vectors (Promega, Madison, Wis.) and sequenced by dideoxy sequencing on an ABI 310 Genetic Analyzer Sequence Analyzer (Applied Biosystems, Foster City, Calif.). Sequences were analyzed with the Wisconsin GCG software package (Accelrys, San Diego, Calif.) and MEGA 3.1 (www megasoftware.net).

TABLE 1a
Pathogens detected in previously negative respiratory specimens

| | | Mass Tag | Real Time | Conventional | |
|---|---|---|---|---|---|
| Sample | Pathogen | PCR | PCR | PCR | Sequencing |
| 3 | HRV NY | + | ND | + | + |
| 4 | Streptococcus pneumoniae | + | ND | + | ND |
| 9 | HCoV-OC43 | + | + | ND | ND |
| 16 | HMPV | + | + | ND | ND |
| | S. pneumoniae | + | ND | ND | + |
| 17 | HMPV | + | + | ND | ND |
| 25 | Mycoplasma pneumoniae | + | + | ND | ND |
| 26 | HRV A | + | ND | + | + |
| 28 | HRV NY | + | ND | + | + |
| 30 | M. pneumoniae | + | + | ND | ND |
| 34 | HRV | + | ND | + | + |
| 37 | HPIV-1 | + | ND | ND | + |
| | S. pneumoniae | + | ND | ND | + |
| | Haemophilus influenzae | + | ND | ND | + |
| 39 | HRV A | + | ND | + | + |
| 41 | HRV NY | + | ND | + | + |
| 42 | HRV NY | + | ND | ND | + |
| 45 | HRV B | + | ND | ND | + |
| | S. pneumoniae | + | ND | ND | + |
| | H. influenzae | + | ND | ND | + |
| 50 | S. pneumoniae | + | ND | + | ND |
| 52 | HEV | + | ND | ND | + |
| 56 | H. influenzae | + | ND | ND | + |
| 60 | HRV NY | + | ND | + | + |
| | S. pneumoniae | + | ND | ND | ND |
| 61 | M. pneumoniae | + | + | ND | ND |
| 63 | HRV NY | + | ND | + | + |
| | S. pneumoniae | + | ND | ND | ND |
| 70 | HCoV-OC43 | + | + | ND | ND |
| 71 | HRSV-B | + | + | ND | ND |
| 72 | M. pneumoniae | + | + | ND | ND |
| 74 | HRV NY | + | ND | + | + |
| 77 | HRV B | + | ND | ND | + |

^{a}HRV, human rhinovirus, (A, group A; B, group B; NY, New York); HCoV-OC43, human coronavirus OC43; HMPV, human metapneumovirus; HPIV-1, human parainfluenza virus 1; HEV, human enterovirus; HRSV-B, human respiratory syncytial virus subgroup B; FLUAV, influenza A; FLUBV, influenza B; HSV, herpes simplex virus.
^{g}cc, conventional viral culture
^{h}nd, not done

TABLE 1b
Additional pathogens in previously positive respiratory specimens

| | | Mass Tag | Real Time | Conventional | | Direct | Conventional |
|---|---|---|---|---|---|---|---|
| Sample | Pathogen | PCR | PCR | PCR | Sequencing | Antigen | Virus Culture |
| 1081 | FLUBV^{b} | + | ND | ND | ND | + | + |
| | HEV | + | + | ND | + | ND | − |
| | S. pneumoniae | + | ND | + | ND | ND | ND |
| 1083 | FLUBV^{b} | + | + | ND | ND | ND | + |
| | HRV B | + | ND | + | + | ND | − |
| 1085 | HRV NY | + | ND | ND | + | ND | − |
| | HSV^{b} | ND | + | ND | ND | ND | + |
| 1101 | FLUAV^{b} | + | + | ND | ND | ND | + |
| | HRV | + | ND | ND | + | ND | − |
| 1119 | FLUBV^{b} | + | ND | ND | ND | + | + |
| | S. pneumoniae | + | ND | ND | + | ND | ND |
| 1126 | FLUAV^{b} | + | ND | ND | ND | + | + |
| | HRV | + | ND | ND | + | ND | − |
| 1135 | FLUAV | + | + | ND | ND | ND | + |
| | FLUAV | | | | | | |
| | N. meningiditis | + | + | ND | ND | ND | ND |
| 1140 | FLUAV^{b} | + | + | ND | ND | + | + |
| | FLUBV^{b} | + | + | ND | ND | − | + |
| | S. pneumoniae | + | ND | + | ND | ND | ND |

^{a}HRV, human rhinovirus, (A, group A; B, group B; NY, New York); HCoV-OC43, human coronavirus OC43; HMPV, human metapneumovirus; HPIV-1, human parainfluenza virus 1; HEV, human enterovirus; HRSV-B, human respiratory syncytial virus subgroup B; FLUAV, influenza A; FLUBV, influenza B; HSV, herpes simplex virus.
Nd: not done
^{b}Previously positive results

### Example 2

### The Rhinovirus Genotype Is Associated With Severe Respiratory Tract Infection In Children In Germany

Acute respiratory infection is a significant cause of morbidity and mortality in children worldwide. Accurate identification of causative agents is important to case management and to prioritization in vaccine development. Sensitive multiplex diagnostics provide an opportunity to investigate the relative contributions of individual agents and may also facilitate pathogen discovery. Application of MassTag PCR to undiagnosed influenza-like illness in New York State led to the discovery of a novel rhinovirus genotype. The invention provides results of a MassTag PCR investigation of pediatric respiratory tract infections in Germany in 97 cases where no pathogen was identified through routine laboratory evaluation. A respiratory virus was identified in 49 cases (51%); rhinoviruses were present in 41 cases (75%). The novel genotype represented 73% of rhinoviruses and 55% of all identified viruses. Infections with the novel genotype were associated with upper respiratory symptoms but more frequently with bronchitis, bronchiolitis, and pneumonia.

Human rhinoviruses (HRVs) are the most frequent cause of acute respiratory illness worldwide. Although HRVs are most commonly associated with mild upper respiratory tract disease (Arruda et al., 1997; Mäkelä et al., 1998; Monto et al., 2002), infection of lower airways does occur (Ketler et al., 1962; Gem et al., 1997; Papadopoulos et al., 2000; Mosser et al., 2005). Lower respiratory tract infections (LRTI) related to HRV are increasingly reported in infants, elderly persons, and immunocompromised patients (Hayden et al., 2004). HRVs are also implicated in exacerbations of asthma (Grissell et al., 2005; Xatzipsalti et al., 2005), chronic bronchitis (Gwatlney, 1989), and acute bronchiolitis (Papadopoulos et al., 2002). Taxonomically, HRVs are currently grouped in two species, Human rhinovirus A and Human rhinovirus B, in the genus Rhinovirus of the family Picornaviridae (ICTVdb http://phene.cpmc.columbia edu; (Fauquet et al., 2005)). These non-enveloped, positive sense single stranded RNA viruses have been classified serologically (Cooney et al., 1982; Hamparian et al., 1987), and on the basis of antiviral susceptibility profile (Andries et al., 1990; Laine et al., 2006), nucleotide sequence relatedness (Hornsell et al., 1995; Savolainen et al., 2002), and receptor usage (intercellular adhesion molecule 1 (ICAM-1), low-density lipoprotein receptor (LDLR), and decay-accelerating factor (DAF)) (Abraham et al., 1984; Uncapher et al., 1991; Blomqvist et al., 2002). Phylogenetic analyses of the VP4/VP2 and the VP 1 coding regions indicated the presence of 76 serotypes in genetic group A and 25 serotypes genetic group B (Laine et al., 2006; Hornsell et al., 1995; Savolainen et al., 2002; Ledford et al., 2004).

Despite the application of PCR assays as well as classical diagnostic methods including culture, antigen tests and serology, an agent is commonly not implicated in up to 50% of cases of severe respiratory disease. Broad-range molecular assay systems such as multiplex PCR (hexaplex (Fan et al., 1998), GeneScan (Erdman et al., 2003), MassTag (Briese et al., 2005)), or microarrays (ViroChip (Wang et al., 2002), panmicrobial GreeneChips (Palacios et al., 2007)) may therefore allow new insights into epidemiology and clinical associations (Lamson et al., 2006; Chiu et al., 2006). With respect to HRV, recent studies employing sensitive PCR systems for these difficult-to-isolate organisms have shown an increased detection rate compared to tissue culture (Arruda et al., 1998; Ireland et al., 1993; Hyypia et al., 1998; Loens et al., 2006; Winther et al., 2006). As described herein, by applying multiplex MassTag PCR platform, numerous agents of respiratory illness were detected in samples that had been submitted for laboratory diagnosis, but had tested negative during routine diagnostic assessment (Lamson et al., 2006). HRVs were identified at high frequency in this sample set. Genetic analysis indicated a large fraction of these viruses to represent a previously uncharacterized genotype of rhinovirus, divergent from groups A (HRV-A) or B (HRV-B).

To gather further information on the potential pathogenicity, as well as temporal and geographic distribution of rhinoviruses, including the recently identified genotype, specimens collected over the 2003 to 2006 seasons from children hospitalized with severe LTR1 in Bad Kreuznach, Germany were evaluated.

Clinical Specimens and Sample Collection

Nasopharyngeal aspirates were obtained from children admitted with acute respiratory tract infection to the Kreuznacher Diakonie Hospital, Bad Kreuznach, Germany, during the interval of 2003 to 2006. Individuals ranged in age from 2 weeks to 5 years (mean 5 months, median 10 months), and 46% were male, 54% female. RNA extraction was performed using QIAamp Viral RNA Kits (Qiagen, Hilden, Germany). Ninety-seven samples that remained without a diagnosed pathogen after assessment by real time reverse transcription—polymerase chain reaction (RT-PCR) assay for influenza virus (Schweiger et al., 2000) and respiratory syncytial virus infection (assay details available upon request) were stored at −70° C. (2003/04 season, n=30; 2004/05 season, n=27; 2005/06 season, n=40).

Assay Procedures

The 97 RNA samples representing cases of undiagnosed respiratory diseases were employed as template for cDNA synthesis by using Superscript II kits with random hexamer priming (Invitrogen, Carlsbad, Calif.), and analyzed in MassTag PCR by using a viral primer panel (Briese et al., 2005), targeting influenzavirus A and B (FLUAV, FLUBV), respiratory syncytial virus A and B (RSV-A, RSV-B), human parainfluenza virus 1, 2, 3, and 4 (HPIV-1, HPIV-2, HPIV-3, HPIV-4), human coronavirus 229E, OC-43 (HCoV-229E, HCoV-OC43), human metapneumovirus (HMPV), enteroand rhinoviruses, and adenoviruses. The fidelity of MassTag PCR signal was substantiated through re-amplification of products and sequence analysis for all positive specimens. In instances where MassTag PCR indicated the presence of a picornavirus, the VP4/2 region was amplified (Coiras et al., 2004). Amplification products were purified from agarose gels and nucleotide sequencing reactions performed on both strands using the ABI Prism Big Dye cycle sequencing kits and ABI Prism Genetic Analyzer systems (Applied Biosytems, Foster City, Calif.). Identical results were obtained with duplicate aliquots processed at the New York and Berlin laboratories. Sequence analyses, alignments, and phylogenetic reconstructions were performed with programs of the Wisconsin GCG Package (Accelrys, San Diego, Calif.) and MEGA 3.1 software (Kumar et al., 2004). Nucleic acid sequences generated during this work are available at GenBank under the following accession numbers: EU081778-EU081816.

Identification of Pathogens

MassTag PCR was used to investigate 97 nasopharyngeal aspirates from children with hospital-admitted, acute respiratory illness wherein no pathogen was identified through routine laboratory testing. At least one candidate respiratory viral pathogen was identified in 49 specimens through MassTag PCR. Although there was variability across the three seasons included in this study, 43% of specimens were positive in the 2003/04 season, 70% of specimens positive in 2004/05, and 43% of specimens positive in 2005/06, picornaviruses represented the majority of identified viruses in each season (Table 2). Nucleic acid sequences were obtained from all MassTag PCR positive specimens for molecular identification. Among the three cases of human adenovirus (HAdV) infection, one HAdV-B, and two HAdVC were identified (Table 2). In case of the picornavirus-positive specimens, one human enterovirus 68 (HEV 68), eight HRV-A, and three HRV-B infections were identified through molecular typing; the remaining 30 sequences (HRV X) did not match with characterized HRV-A, HRV-B, or HEV sequences.

TABLE 2
Viral pathogens detected by MassTag PCR in children hospitalized with respiratory disease

| | Season | | |
|---|---|---|---|
| Category | 2003-2004 | 2004-2005 | 2005-2006 |
| Positive cases, proportion (%) | 13/30 (43% | 19/27 (70% | 17/40 (43% |
| Pathogens detected (no.) | HPIV-2 (1) | RSV-B (1) | HPIV-1 (3) |
| | HAdV (1) | HPIV-3 (1) | HPIV-2 (1) |
| | HEV/HRV (12) | HPIV-4 (1) | HMPV (1) |
| | | HCoV-OC43 (1) | HEV/HRV (15) |
| | | HAdV (2) | |
| | | HEV/HRV (15) | |
| Specific identification (no.) | HAdV-B (1) | HAdV-C (2) a | HRV-A (2) |
| | HRV-A (3) | HEV-68 (1) | HRV X (13) |
| | HRV-B (3) | HRV-A (3) | |
| | HRV X b (6) | HRV X (11) | |
| Coinfections (no.) | HRV-B/HRV X (1) | HAdV-C/HRV X (1) | HPIV-1/HRV X (1) |
| | | HAdV-C/HPIV-3 (1) | HMPV/HRV X (1) |
| | | | HRV-A/HRV X (1) |

NOTE.
HAdV, human adenovirus; HCoV, human coronavirus; HMPV, human metapneumovirus; HPIV, human parainfluenza virus; RSV, respiratory syncytial virus.
a Sequence is related to type 1 (1) or type 2 (1).
b The remaining 30 human rhinovirus sequences (HRV X) did not match up with the characterized HRV-A, HRV-B, or human enterovirus (HEV) sequences; for 30 of the VP4/VP2 sequences, analysis at the nucleotide level, with the use of the Basic Local Alignment Search Tool, did not indicate a significant match with HRV-A, HRV-B, or HEV sequences, and analysis at the amino-acid level revealed homology to entero- and rhinoviral sequences with a sequence identity of 60%-65%

Clinical Associations

HRVs were the most frequent viruses detected in the sample set, representing 75% (41/55) of the identified viruses. Co-infection with another virus was observed in only 12% (5/41) of these cases (Table 3). Fever or cough were recorded with similar frequency in infections with HRVs (82%) and the other viruses (89%). Rhinitis or pharyngitis were more often observed with HRV (79%) than with the other virus infections (56%). The frequency of lower respiratory symptoms (bronchitis, bronchiolitis, pneumonia) was comparable in HRV (71%) and other viral infections (67%). Whereas pneumonia was more common with HRV-A/B (56%) than with HRV X (36%) infections, the frequencies of bronchiolitis (HRV-A/B, 11%; HRV X, 12%) and bronchitis (HRV-A/B, 67%; HRV X, 60%) were similar. LRTI was recorded in 72% of HRV X infections; however, some cases were related to milder disease (Table 3).

Molecular Epidemiology of Identified Picornaviruses

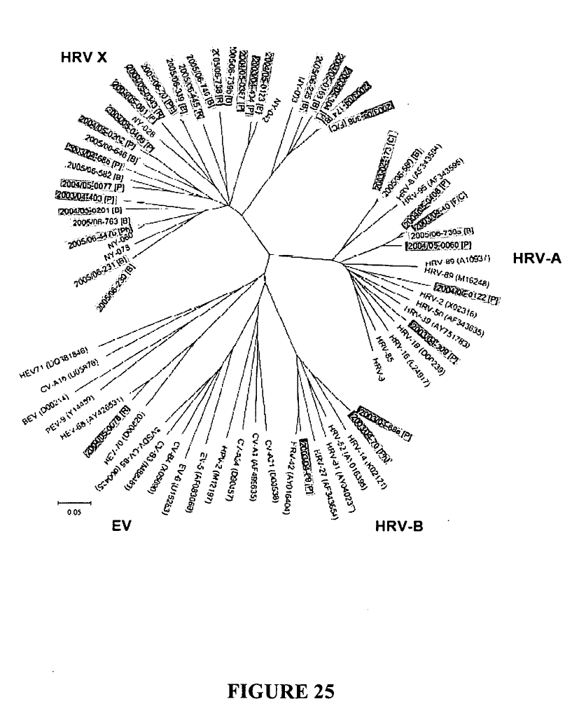

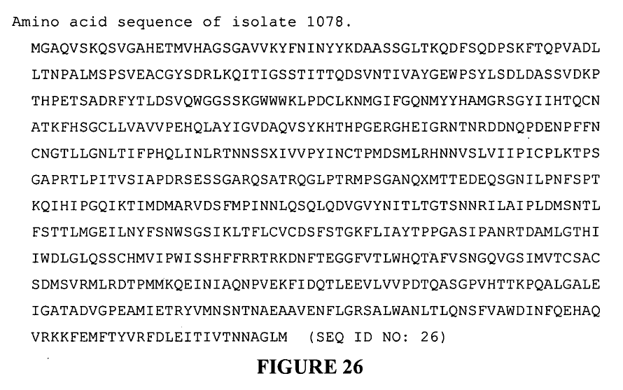

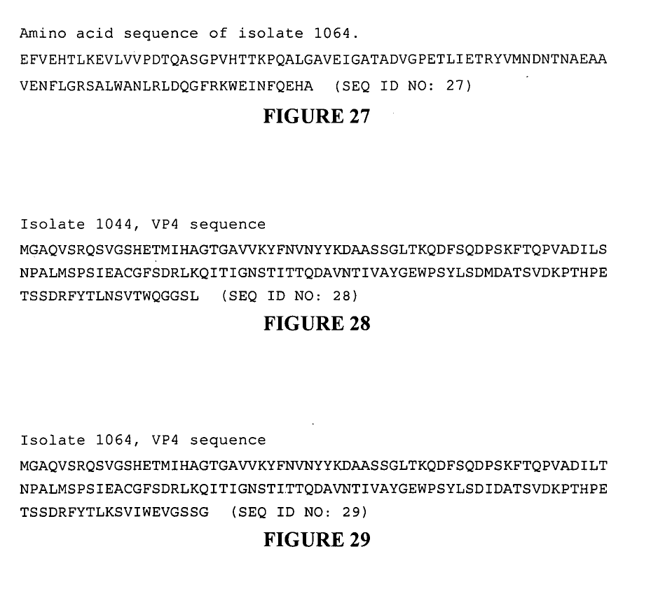

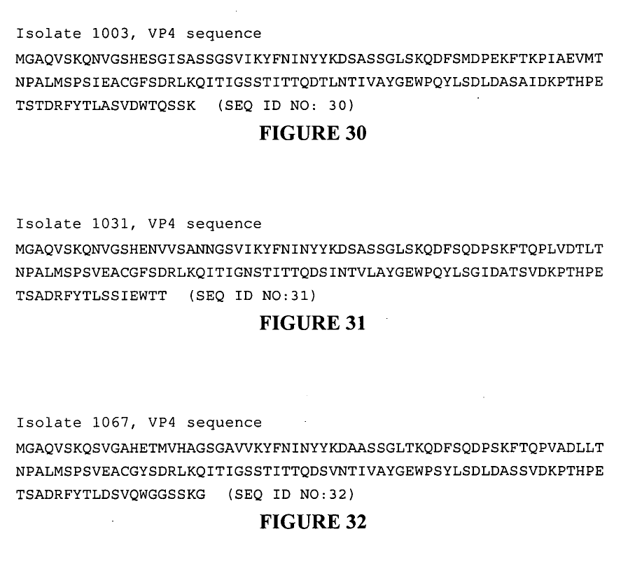

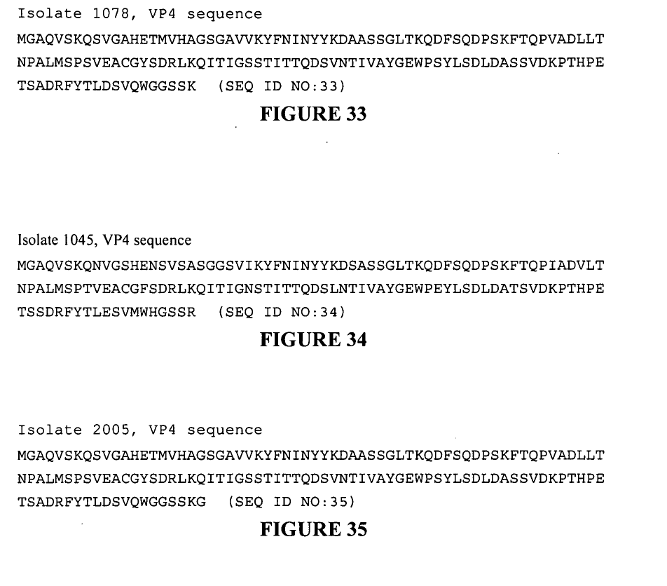

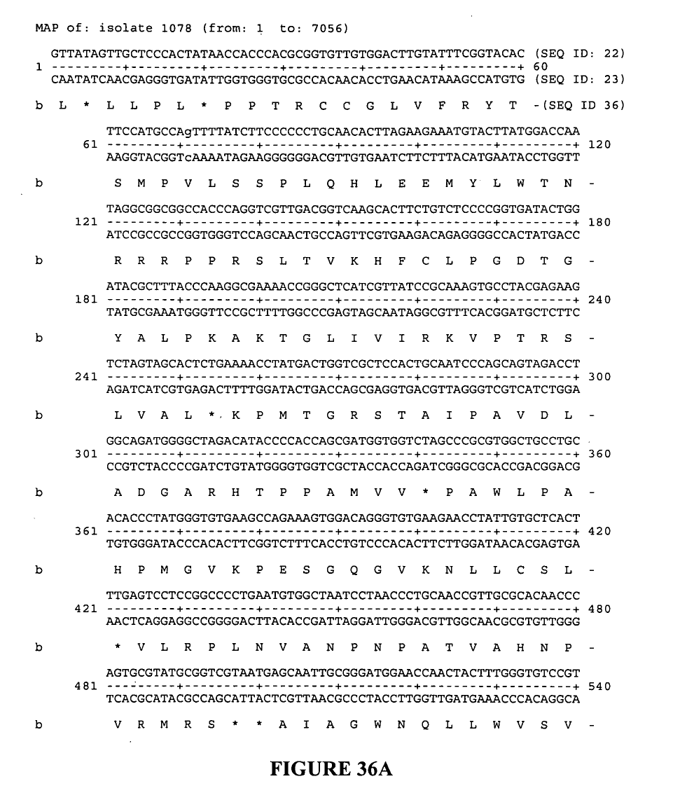

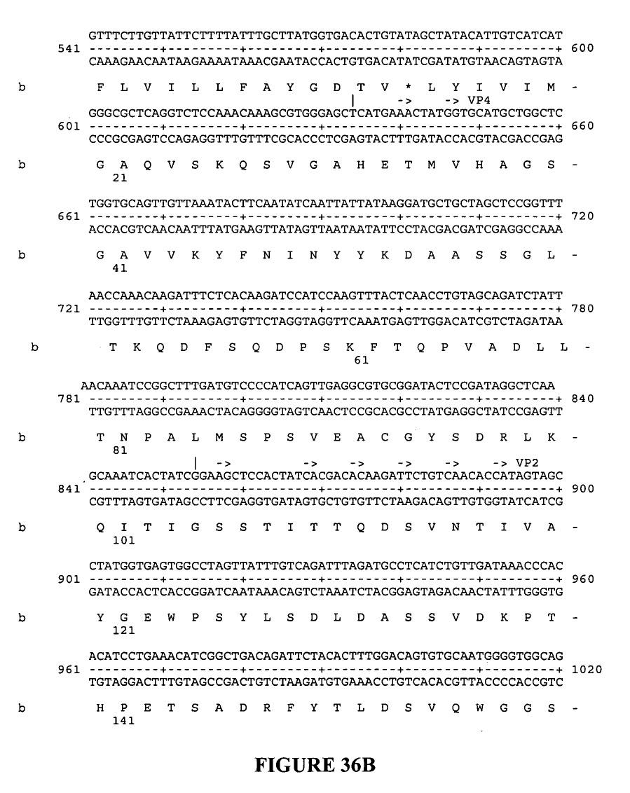

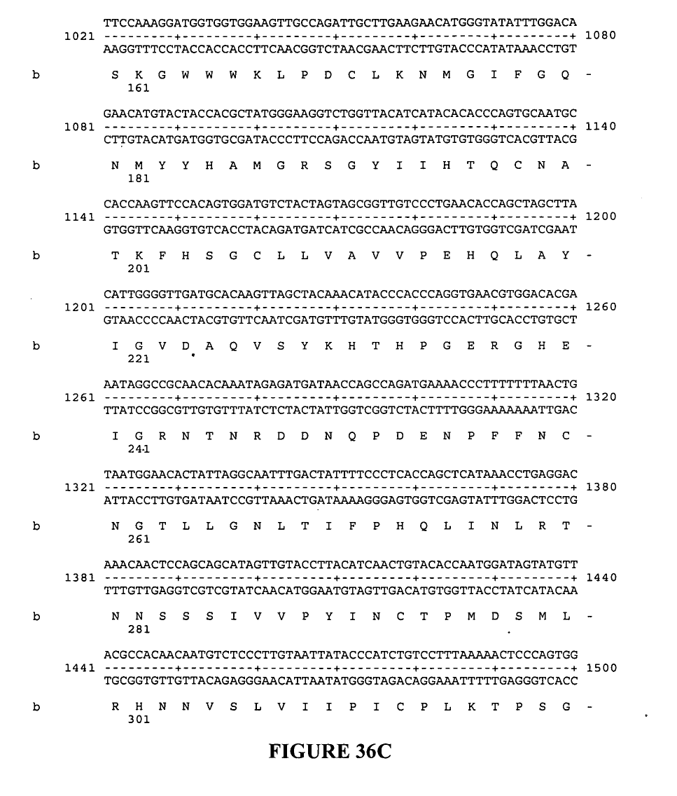

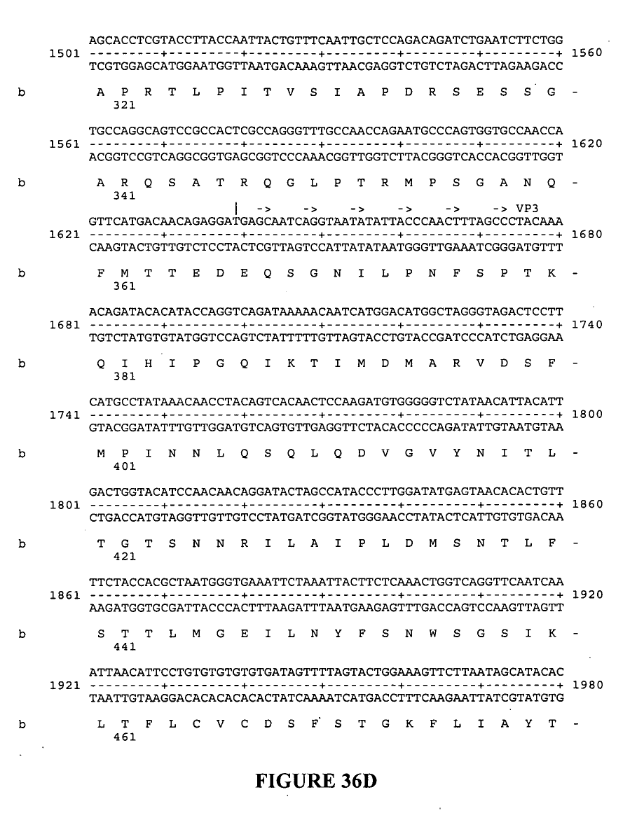

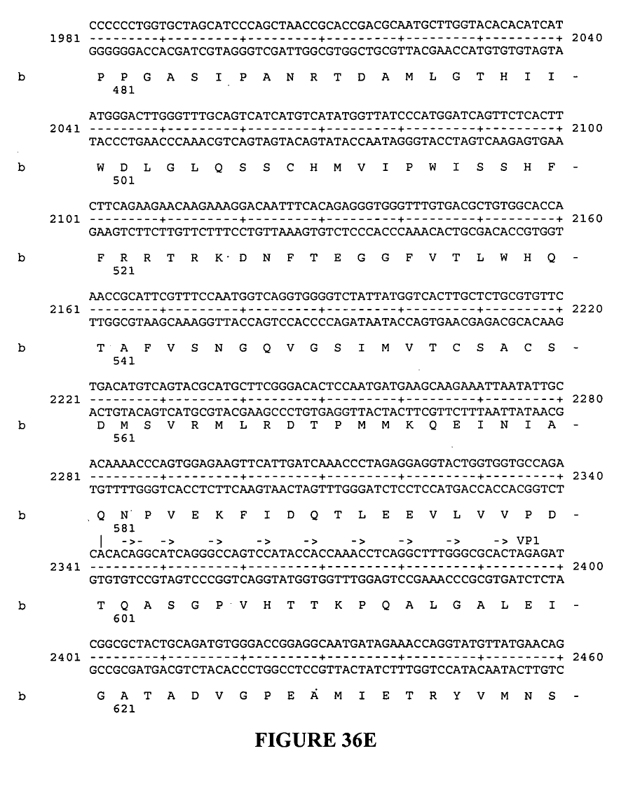

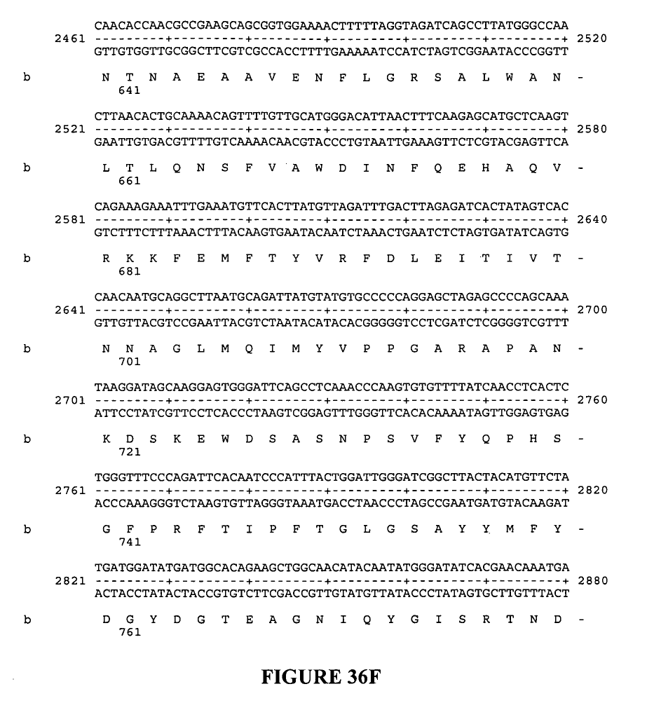

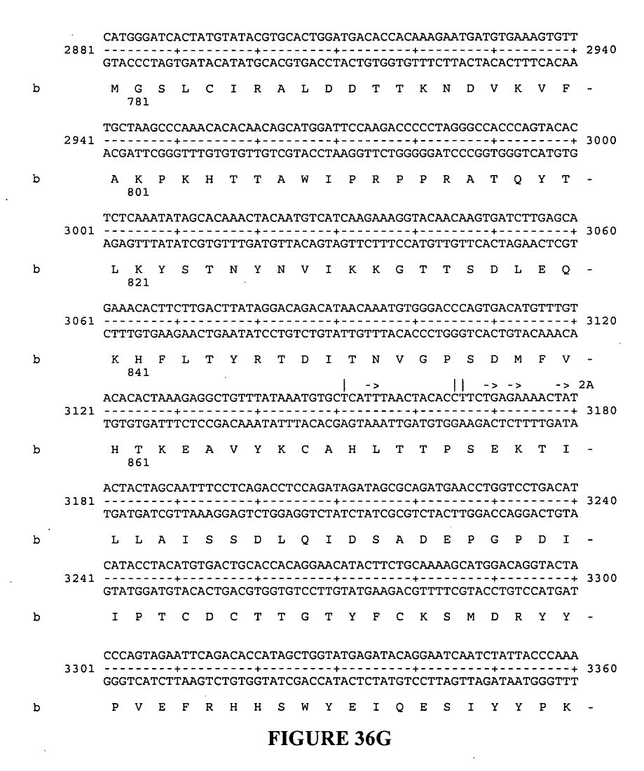

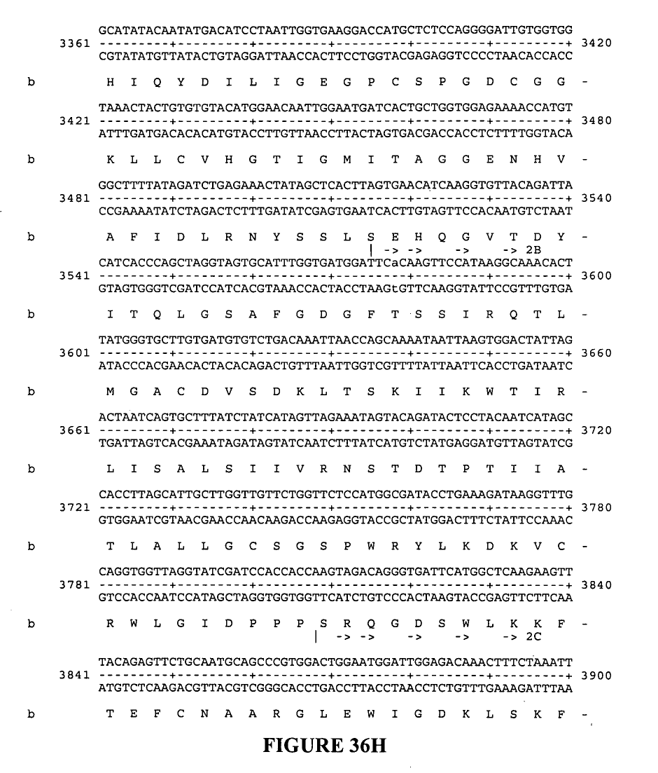

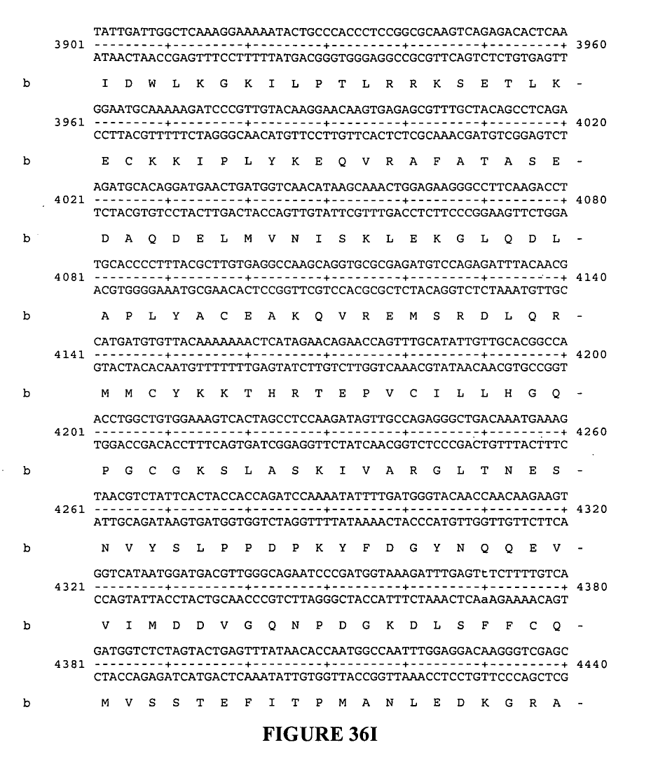

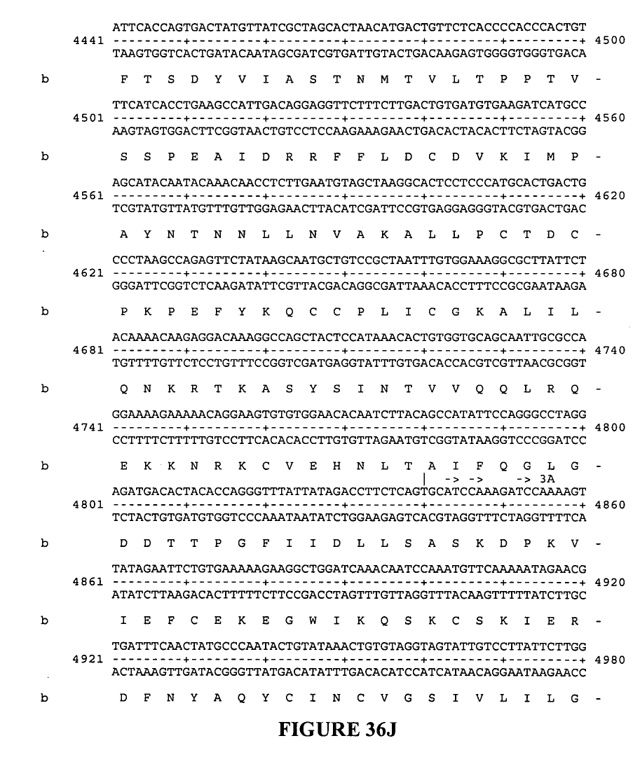

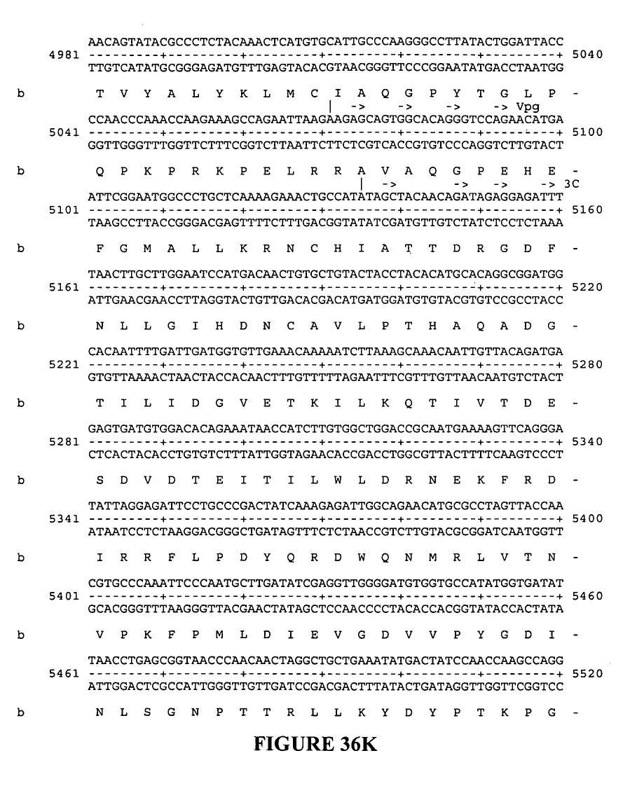

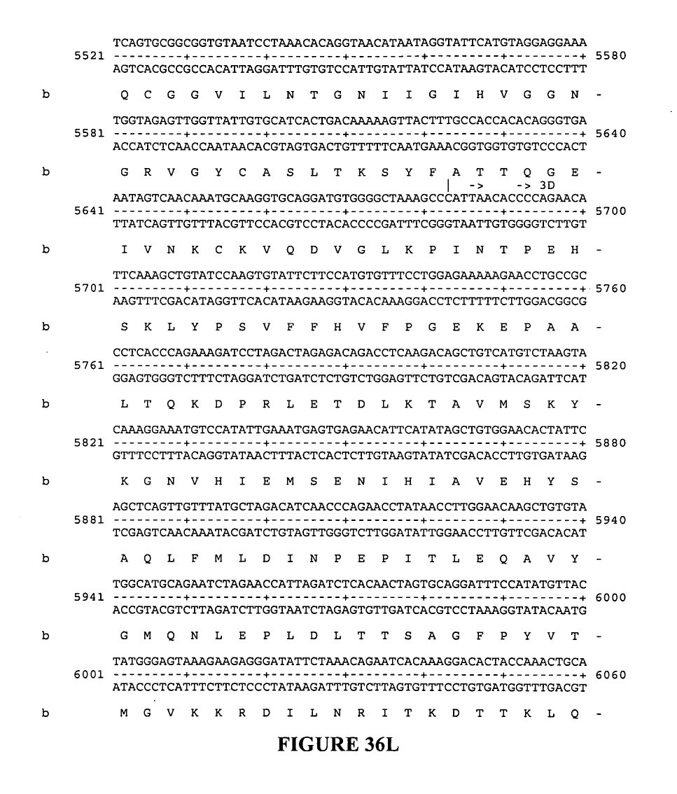

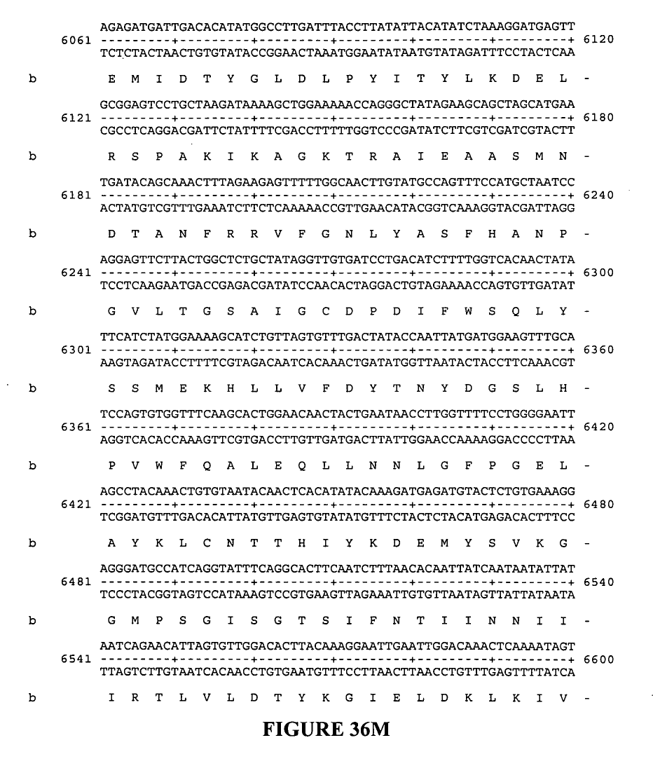

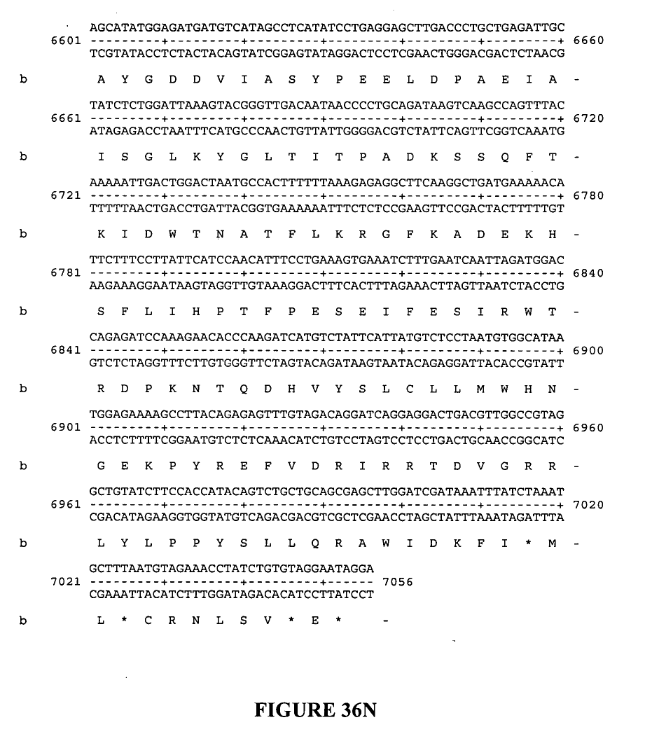

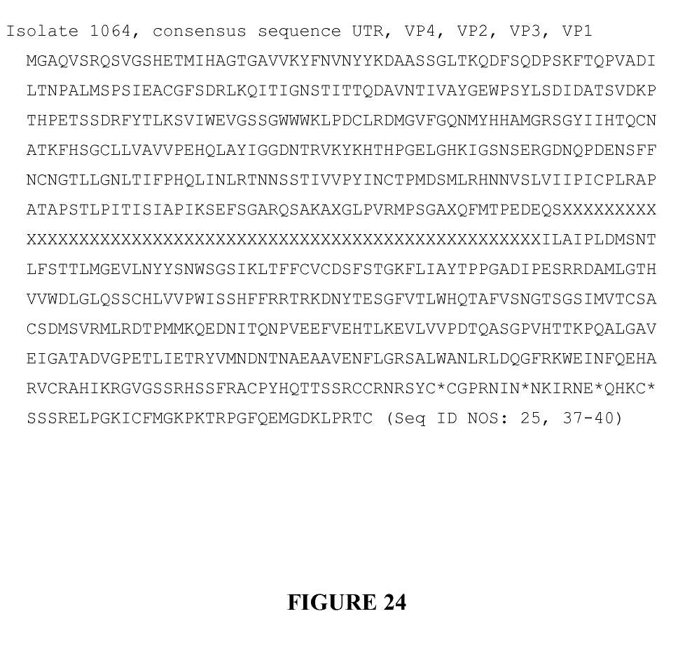

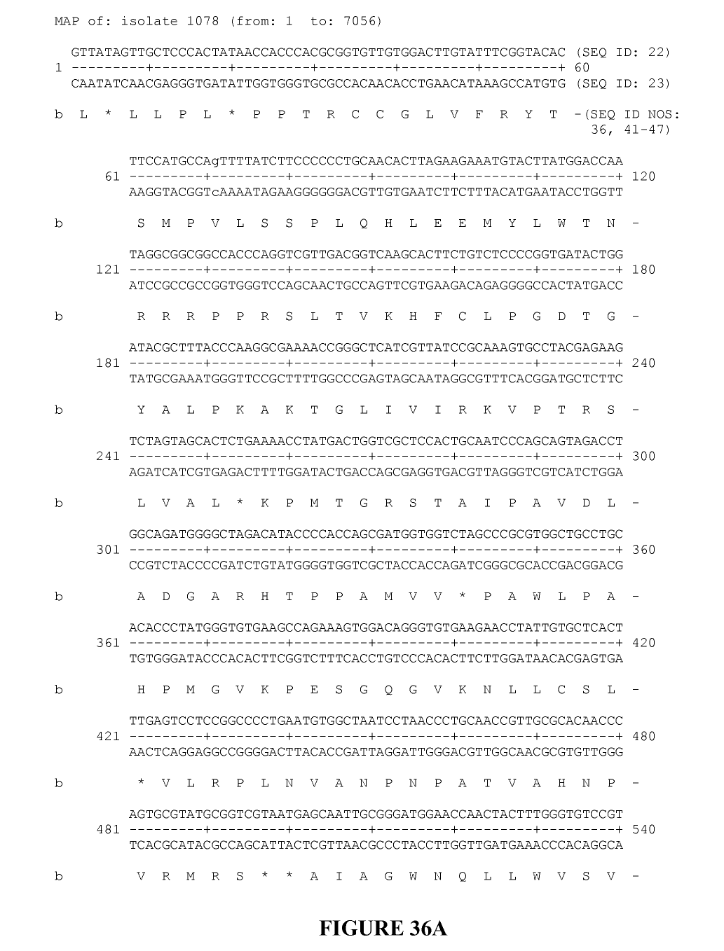

MassTag PCR targets conserved sequences in the 5′-untranslated region of entero- and rhinoviruses; thus, to facilitate phylogenetic analysis of HEV and HRVs, the VP4/2 gene region was amplified and sequenced. However, Basic Local Alignment Search Tool (BLAST) analysis at the nucleotide level did not indicate a significant match with HRV-A, HRV-B, or HEV sequences for 30 of the VP4/2 sequences; analysis at the amino acid level revealed homology to entero- and rhinoviral sequences with a sequence identity of 60-65%. High similarity at both the nucleotide and amino acid levels was evident when sequences were aligned with an unclassified genetic clade of picornaviruses recently identified in New York State (Lamson et al., 2006). However, detailed phylogenetic analysis indicated significant sequence diversity among the 30 viruses (FIG. 25). Temporal analysis over three seasons indicated a lower frequency of the novel genotype in the 2003 season (20%, 6/30) compared to the 2004 (41%, 11/27) or 2005 (33%, 13/40) seasons; phylogenetic clustering by season was not obvious. No significant relation was observed between the HRV genotypes and clinical diagnoses (FIG. 25).

In this study of samples collected over a three-year interval from hospitalized children with severe undiagnosed respiratory infection, MassTag PCR allowed detection of viral pathogens in 49 of 97 cases (51%). The pathogens most commonly identified were HRVs. These findings are consistent with other studies which indicate that rhinoviruses, or picornaviruses, account for 20-80% of acute respiratory infections

Arruda et al., 1997; Ireland et al., 1993; Hyypia et al., 1998; Johnston et al., 1993; Nokso-Koivisto et al., 2002; Jartti et al., 2004), exceeding in some instances even the frequency of RSV infection in pediatric patient populations (Loens et al., 2006; Jartti et al., 20041 Rakes et al., 1999; Miller et al., 2007). The presence of HRV is not sufficient to prove causation. Asymptomatic HRV infection has been described; however, the extent to which infection without disease represents carriage, incubation or convalescence is unknown (Winther et al., 2006; Johnston et al., 1993; Nokso-Koivisto et al., 2002; Rakes et al., 1999; Jartti et al., 2004). The high frequency of HRV as the sole virus detected suggests a correlation between the agent and the observed LRTI symptoms even though there were no samples to test for the presence of HRV in lower respiratory tract. Support for HRV as pathogenic in LRTI comes from the findings that HRVs have been demonstrated in lower airways by in situ hybridization, and have been shown to trigger inflammatory processes in the infected cells and tissues (Gem et al., 1997; Papadopoulos et al, 2000; Mosser et al., 2005; Fraenkel et al., 1995; Johnston et al., 1998; Schroth et al, 1999; Seymour et al., 2002).

Among the HRVs identified in this study were representatives of the novel genetic clade recently discovered in New York State (Lamson et al., 2006); indeed, these viruses were the majority of HRVs detected. HRV-A and HRV-B have been implicated in common colds as well as severe LRTI. Viruses of the novel genetic clade were also associated in the patients with a wide range of disease ranging from rhinitis to bronchitis and severe pneumonia necessitating supplemental oxygen in about 50% of cases. A seasonal pattern of HRV infections has been described (Mäkelä et al., 1998; Monto et al., 2002; Miller et al, 2007); however, data regarding serotype- or genotype-specific patterns of seasonality or disease symptoms are limited (Calhoun et al., 1974; Fox et al., 1975; Roebuck, 1976). A temporal trend of sequence diversity or correlation of genotypes within the novel HRV clade with clinical diagnosis was not apparent in the data set (FIG. 25).

No detailed information is yet available concerning the history of the novel HRV clade; nonetheless, the sequence diversity observed within the clade is not consistent with recent introduction. This clade may account in part for earlier reports of non-typeable rhinoviruses (Jartti et al., 2004; Jartti et al., 2004). Indeed, its discovery may reflect the implementation of new technologies rather than novelty per se. Additional analysis using the methods described herein can be used to define more than one serogroup. The results described herein indicate the significance of HRVs in pediatric LRTI. The presence of novel HRVs in two disparate geographic locations in association with serious respiratory disease in children as well as adults mandates further work in epidemiology and pathogenesis.

TABLE 3
Patient and Clinical Data

| | | | | | | | | | | | | | Miscellaneous |
|---|---|---|---|---|---|---|---|---|---|---|---|---|---|
| Season, | | | | | | | Rhi- | Pharyn- | Laryn- | Bron- | Bronchi- | Pneu- | symptoms/ |
| individual | Age | | Sex | Agent | Fever ° C. | Cough | nitis | gitis | gitis | chitis | olitis | monia | remarks |
| 2003-2004 | | | | | | | | | | | | | |
| 40 | 11 | months | M | HRV-A | + | + | − | − | − | − | − | − | Chills |
| 68 | 11 | months | M | HRV-B/ | − | + | + | + | − | + | − | + | Otitis |
| | | | | HRV X | | | | | | | | | |
| 69 | NA | | M | HRV-B | − | + | − | + | − | + | − | + | Conjunctivitis |
| 70 | 1 | month | M | HRV-B | >39 | − | + | + | − | − | − | − | Otitis |
| 172 | NA | | M | HPIV-2 | − | + | − | − | − | − | − | − | |
| 173 | 2 | years | F | HRV-A | − | + | − | − | − | − | − | − | Outpatient |
| 174 | 2 | months | F | HRV X | − | + | + | + | − | + | − | − | |
| 304 | 12 | months | F | HRV X | − | − | + | + | − | − | − | + | |
| 306 | NA | | F | HRV X | + | + | − | − | − | − | − | − | Chills, |
| | | | | | | | | | | | | | outpatient |
| 309 | 12 | months | M | HRV-A | >39 | + | + | + | − | + | − | + | Chills |
| 314 | 5 | years | F | HAdV-B | + | + | − | − | − | − | − | + | Chills |
| 403 | 3 | months | M | HRV X | <39 | + | + | + | − | − | − | + | Conjunctivitis |
| 404 | 7 | months | M | HRV X | + | + | + | + | − | + | − | + | Chills |
| 2004-2005 | | | | | | | | | | | | | |
| 0060 | 12 | months | F | HRV-A | >39 | + | + | + | − | + | + | + | |
| 0061 | 8 | months | M | HRV X | − | + | + | + | − | + | + | + | |
| 0077 | NA | | F | HRV X | − | + | + | − | − | + | − | + | |
| 0078 | 2 | months | M | HEV-D | − | − | + | − | − | − | − | − | |
| 0121 | 2 | years | F | HPIV-4 | >39 | + | − | − | − | − | − | − | Chills |
| 0122 | 13 | months | F | HRV-A | <39 | + | + | + | − | + | − | + | |
| 0123 | 4 | months | F | HRV X | − | − | − | + | − | + | − | − | Gastroenteritis |
| 0162 | 17 | months | F | HCoV- | + | + | + | + | − | − | − | + | Chills, tonsillitis |
| | | | | OC43 | | | | | | | | | |
| 0163 | 1 | month | F | HRV X | <39 | + | + | + | − | + | − | − | Conjunctivitis |
| 0201 | 7 | months | NA | HAdV-C/ | <39 | + | + | − | − | + | − | − | |
| | | | | HRV X | | | | | | | | | |
| 0202 | 4 | months | M | HRV X | − | + | + | + | − | + | − | + | |
| 0203 | 14 | months | F | HRV X | − | + | + | + | − | − | − | − | |
| 0269 | 13 | months | M | HPIV-3/ | − | − | − | + | − | − | + | − | |
| | | | | HAdV-C | | | | | | | | | |
| 0282 | 7 | months | F | RSV-B | − | + | + | − | − | + | − | − | |
| 0335 | 24 | months | M | HRV X | >39 | + | + | + | − | + | − | + | |
| 0343 | 2 | weeks | F | HRV X | >39 | + | + | − | − | − | − | − | Chills, |
| | | | | | | | | | | | | | gastroenteritis |
| 0367 | 2 | weeks | M | HRV X | >39 | − | + | − | − | − | − | + | |
| 0408 | 7 | months | F | HRV-A | <39 | + | + | + | − | + | − | + | Otitis |
| 0409 | 7 | months | M | HRV X | <39 | + | + | + | − | + | + | + | Otitis |
| 2005-2006 | | | | | | | | | | | | | |
| 020 | 2 | months | F | HRV X | − | + | + | + | − | − | − | − | Outpatient |
| 225 | 12 | months | F | HRV X | − | + | + | + | − | + | − | − | |
| 230 | 11 | months | M | HRV X | − | + | + | − | − | + | − | − | |
| 231 | 3 | months | F | HPIV-1/ | <39 | + | + | + | − | + | − | − | |
| | | | | HRV X | | | | | | | | | |
| 325 | 12 | months | F | HPIV-2 | >39 | + | + | + | − | + | − | − | Chills, otitis, |
| | | | | | | | | | | | | | gastroenteritis |
| 339 | 12 | months | M | HRV X | + | + | − | − | − | + | − | − | Chills, |
| | | | | | | | | | | | | | head/muscle |
| | | | | | | | | | | | | | pain |
| 445 | 1 | month | F | HRV X | − | − | + | − | − | − | − | − | |
| 446 | 12 | months | M | HPIV-1 | <39 | + | − | + | + | + | − | − | Tonsillitis |
| 447 | 2 | months | F | HRV X | − | − | + | + | − | − | − | − | Tonsillitis |
| 580 | 2 | months | M | HRV-A | − | − | − | − | − | + | − | − | |
| 582 | 6 | months | F | HRV X | + | + | − | − | − | + | − | − | Chills |
| 646 | 12 | months | M | HRV X | <39 | + | − | + | − | + | − | − | Chills |
| 673 | 12 | months | F | HPIV-1 | + | + | − | − | − | + | − | − | Chills |
| 738 | 11 | months | F | HRV X | − | − | + | − | − | − | − | − | |
| 739 | 3 | months | F | HRV-A/ | >39 | + | + | + | − | + | − | − | |
| | | | | HRV X | | | | | | | | | |
| 740 | 5 | months | M | HMPV/ | <39 | + | − | − | − | + | − | − | Outpatient |
| | | | | HRV X | | | | | | | | | |
| 763 | 12 | months | M | HRV X | − | − | − | − | − | + | + | − | |

NOTE.
HAdV, human adenovirus; HCoV, human coronavirus; HEV, human enterovirus; HMPV, human metapneumovirus; HPIV, human parainfluenza virus; HRV, human rhinovirus; NA, not available; RSV, respiratory syncytial virus; _, presence; _, absence.

### REFERENCES

Abraham G, Colonno R J. Many rhinovirus serotypes share the same cellular receptor. J Virol 1984; 51:340-5.
Andries K, Dewindt B, Snoeks J, et al. Two groups of rhinoviruses revealed by a panel of antiviral compounds present sequence divergence and differential pathogenicity. J Virol
Arruda E, Pitkaranta A, Witek T J, Jr., Doyle C A and Hayden F G. Frequency and natural history of rhinovirus infections in adults during autumn. J Clin Microbiol 1997; 35:2864-8
Blomqvist S, Savolainen C, Raman L, Roivainen M and Hovi T. Human rhinovirus 87 and enterovirus 68 represent a unique serotype with rhinovirus and enterovirus features. J Clin Microbiol 2002; 40:4218-23
Briese T, Palacios G, Kokoris M, et al. Diagnostic system for rapid and sensitive differential detection of pathogens. Emerg Inf Dis 2005; 11:310-3. Briese T, Renwick N, Venter M, Jarman R G, Ghosh D, Kondgen S, Shrestha S K, Hoegh A M, Casas I, Adjogoua E V, Akoua-Koffi C, Myint K S, Williams D T, Chidlow G, van den Berg R, Calvo C, Koch O, Palacios G, Kapoor V, Villari J, Dominguez S R, Holmes K V, Harnett G, Smith D, Mackenzie J S, Ellerbrok H, Schweiger B, Schonning K, Chadha M S, Leendertz F H, Mishra A C, Gibbons R V, Holmes E C, Lipkin W I. Global distribution of novel rhinovirus genotype. Emerg Infect Dis. 2008 June; 14(6):944-7.
Calhoun A M, Jordan W S, Jr. and Gwaltney J M, Jr. Rhinovirus infections in an industrial population. V. Change in distribution of serotypes. Am J Epidemiol 1974; 99:58-64
Chiu C Y, Rouskin S, Koshy A, et al. Microarray detection of human parainfluenzavirus 4 infection associated with respiratory failure in an immunocompetent adult. Clin Infect Dis 2006; 43:e71-6.
Coiras M T, Aguilar J C, Garcia M L, Casas I and Perez-Brena P. Simultaneous detection of fourteen respiratory viruses in clinical specimens by two multiplex reverse transcription nested-PCR assays. J Med Virol 2004; 72:484-95
Cooney M K, Fox J P and Kenny G E. Antigenic groupings of 90 rhinovirus serotypes. Infect Immun 1982; 37:642-7
Corless C E, Guiver M, Borrow R, Edwards-Jones V, Fox A J, Kaczmarski E B. Simultaneous detection of Neisseria meningitides, Haemophilus influenzae, and Streptococcus pneumoniae in suspected cases of meningitis and septicemia using real-time PCR. J Clin Microbiol 2001 39; 1553-8.
Dominguez S R, Briese T, Palacios G, Hui J, Villari J, Kapoor V, Tokarz R, Glod& M P, Anderson M S, Robinson C C, Holmes K V, Lipkin W I. Multiplex MassTag-PCR for respiratory pathogens in pediatric nasopharyngeal washes negative by conventional diagnostic testing shows a high prevalence of viruses belonging to a newly recognized rhinovirus clade. J Clin Virol. 2008 October; 43(2):219-22.
Erdman D D, Weinberg G A, Edwards K M, et al. GeneScan reverse transcription—PCR assay for detection of six common respiratory viruses in young children hospitalized with acute respiratory illness. J Clin Microbiol 2003; 41:4298-303
Fan J, Henrickson K J and Savatski L L. Rapid simultaneous diagnosis of infections with respiratory syncytial viruses A and B, influenza viruses A and B, and human parainfluenza virus types 1, 2, and 3 by multiplex quantitative reverse transcription polymerase chain reaction-enzyme hybridization assay (Hexaplex). Clin Infect Dis 1998; 26: 1397-402
Fauquet M F, Mayo M A, Maniloff J, Desselberger U and Ball L A. Virus Taxonomy: Eighth Report of the International Committee on Taxonomy of Viruses. London: Elsevier Academic Press, 2005
Fox J P, Cooney M K and Hall C E. The Seattle virus watch. V. Epidemiologic observations of rhinovirus infections, 1965-1969, in families with young children. Am J Epidemiol
Fraenkel D J, Bardin P G, Sanderson G, Lampe F, Johnston S L and Holgate S T. Lower airways inflammation during rhinovirus colds in normal and in asthmatic subjects. Am J Respir Crit. Care Med 1995; 151:879-86
Gem J E, Galagan D M, Jarjour N N, Dick E C and Busse W W. Detection of rhinovirus RNA in lower airway cells during experimentally induced infection. Am J Respir Crit. Care Med 1997; 155:1159-61
Grissell T V, Powell H, Shafren D R, et al. Interleukin-10 gene expression in acute virus-induced asthma. Am J Respir Crit. Care Med 2005; 172:433-9
Gwaltney J M, Jr. Rhinoviruses. In: Evans AS, ed. Viral infections of human. New York: Plenum Medical Book Co, 1989:593-615
Hamparian V V, Colonno R J, Cooney M K, et al. A collaborative report: rhinoviruses—extension of the numbering system from 89 to 100. Virology 1987; 159:191-2
Hassan-King M, Baldeh I, Secka O, Falade A, Greenwood B. Detection of Streptococcus pneumoniae DNA in blood cultures by PCR. J Clin Microbiol 1994; 32:1721-4.
Hayden F G. Rhinovirus and the lower respiratory tract. Rev Med Virol 2004; 14:17-31
Horsnell C, Gama R E, Hughes P J and Stanway G. Molecular relationships between 21 human rhinovirus serotypes. J Gen Virol 1995; 76 (Pt 10):2549-55
Hyypiä T, Puhakka T, Ruuskanen O, Mäkela M, Arola A and Arstila P. Molecular diagnosis of human rhinovirus infections: comparison with virus isolation. J Clin Microbiol
Ireland D C, Kent J and Nicholson K G. Improved detection of rhinoviruses in nasal and throat swabs by seminested RT-PCR. J Med Virol 1993; 40:96-101
Ishizuka S, Yamaya M, Suzuki T, et al. Effects of rhinovirus infection on the adherence of Streptococcus pneumoniae to cultured human airway epithelial cells. J Inf Dis 2003; 188: 1928-39.
Jartti T, Lehtinen P, Vuorinen T, et al. Respiratory picornaviruses and respiratory syncytial virus as causative agents of acute expiratory wheezing in children. Emerg Infect Dis
Jartti T, Lehtinen P, Vuorinen T, Koskenvuo M and Ruuskanen O. Persistence of rhinovirus and enterovirus RNA after acute respiratory illness in children. J Med Virol 2004; 72:695-9
Johnston S L, Papi A, Bates P J, Mastronarde J G, Monick M M and Hunninghake G W. Low grade rhinovirus infection induces a prolonged release of IL-8 in pulmonary epithelium. J Immunol 1998; 160:6172-81
Johnston S L, Sanderson G, Pattermore P K, et al. Use of polymerase chain reaction for diagnosis of picornavirus infection in subjects with and without respiratory symptoms. J Clin Microbiol 1993; 31:111-7.
Ketler A, Hamparian V V and Hilleman M R. Characterization and classification of ECHO 28-rhinovirus-coryzavirus agents. Proc Soc Exp Biol Med 1962; 110:821-31
Khanna M, Fan J, Pehler-Harrington K, et al. The Pneumoplex assays, a multiplex PCR-enzyme hybridization assay that allows simultaneous detection of five organisms, Mycoplasma pneumoniae, Chlamydia (Chlamydophila) pneumoniae, Legionella pneumophila, Legionella micdadei, and Bordetella pertussis, and its real-time counterpart. J Clin Microbiol 2005; 43: 565-71.
Kumar S, Tamura K and Nei M. MEGA3: Integrated software for Molecular Evolutionary Genetics Analysis and sequence alignment. Brief Bioinform 2004; 5: 150-63
Laine P, Blomqvist S, Savolainen C, Andries K and Hovi T. Alignment of capsid protein VP1 sequences of all human rhinovirus prototype strains: conserved motifs and functional domains. J Gen Virol 2006; 87: 129-38
Lamson D, Renwick N, Kapoor V, et al. MassTag polymerase-chain-reaction detection of respiratory pathogens, including a new rhinovirus genotype, that caused influeza-like illness in New York State during 2004-2005. J. Inf. Dis. 2006; 194:1398-1402.
Ledford R M, Patel N R, Demenczuk T M, et al. VP1 sequencing of all human rhinovirus serotypes: insights into genus phylogeny and susceptibility to antiviral capsid-binding compounds. J Virol 2004; 78:3663-74
Loens K, Goosens H, de Laat C, et al. Detection of rhinoviruses by tissue culture and two independent amplification techniques, nucleic acid sequence-based amplification and reverse transcription-PCR, in children with acute respiratory infections during a winter season. J Clin Microbiol 2006; 44:166-71.

Mäkelä M J, Puhakka T, Ruuskanen O, et al. Viruses and bacteria in the etiology of the common cold. J Clin Microbiol 1998; 36:539-42
Miller E K, Lu X, Erdman D D, et al. Rhinovirus-associated hospitalizations in young children. J Infect Dis 2007; 195:773-81
Monto A S. The seasonality of rhinovirus infections and its implications for clinical recognition. Clin Ther 2002; 24:1987-97
Mosser A G, Vrtis R, Burchell L, et al. Quantitative and qualitative analysis of rhinovirus infection in bronchial tissues. Am J Respir Crit. Care Med 2005; 171:645-51
Nix W A, Oberste M S, Pallansch M A. Sensitive, semi-nested PCR amplification of VP 1 sequences for direct identification of all enterovirus serotypes from original clinical specimens. J Clin Microbiol, 2006 August; 44(8):2698-704.
Nokso-Koivisto J, Kinnari T J, Lindahl P, Hovi T and Pitkaranta A. Human picornavirus and coronavirus RNA in nasopharynx of children without concurrent respiratory symptoms. J Med Virol 2002; 66:417-20
Palacios G, Briese T, Kapoor V, et al. MassTag Polymerase Chain Reaction for differential detection of viral hemorrhagic fevers. Emerg Inf Dis 2006; 12:692-5.
Palacios G, Quan P-L, Jabado O J, et al. Panmicrobial oligonucleotide array for diagnosis of infectious diseases. Emerg Inf Dis 2007; 13:73-81
Papadopoulos N G, Bates P J, Bardin P G, et al. Rhinoviruses infect the lower airways. J Infect Dis 2000; 181:1875-84
Papadopoulos N G, Moustaki M, Tsolia M, et al. Association of rhinovirus infection with increased disease severity in acute bronchiolitis. Am J Respir Crit. Care Med 2002; 165:1285-9.
Rakes G P, Arruda E, Ingram J M, et al. Rhinovirus and respiratory syncytial virus in wheezing children requiring emergency care. IgE and eosinophil analyses. Am J Respir Crit. Care Med 1999; 159:785-90

Renwick N, Schweiger B, Kapoor V, Liu Z, Villari J, Bullmann R, Miething R, Briese T, Lipkin W I. A recently identified rhinovirus genotype is associated with severe respiratory-tract infection in children in Germany. J Infect Dis. 2007 Dec. 15; 196(12):1754-60.
Roebuck M O. Rhinoviruses in Britain 1963-1973. J Hyg 1976; 76: 137-146
Savolainen C, Blomqvist S, Mulders M N and Hovi T. Genetic clustering of all 102 human rhinovirus prototype strains: serotype 87 is close to human enterovirus 70. J Gen Virol 2002; 83:333-40.
Schoub B, Martin D. Influenza Pandemic Preparedness. World Health Organization. Apr. 12, 2006. <www.who.int/entity/csr/disease/influenza/southafricaplan pdf>
Schroth M K, Grimm E, Frindt P, et al. Rhinovirus replication causes RANTES production in primary bronchial epithelial cells. Am J Respir Cell Mol Biol 1999; 20: 1220-8
Schweiger B, Zadow I, Heckler R, Timm H and Pauli G. Application of a fluorogenic PCR assay for typing and subtyping of influenza viruses in respiratory samples. J Clin Microbiol 2000; 38:1552-8
Seymour M L, Gilby N, Bardin P G, et al. Rhinovirus infection increases 5-lipoxygenase and cyclooxygenase-2 in bronchial biopsy specimens from nonatopic subjects. J Infect Dis 2002; 185:540-4
Syrmis M W, Whiley D M, Thomas M, et al. A sensitive, specific and cost-effective multiplex reverse transcriptase-PCR assay for the detection of seven common respiratory viruses in respiratory samples. J Mol Diagn 2004; 6:125-31.
Templeton K E, Schelting a S A, Beersma M F C, Kroes A C M, Claas E C J. Rapid and sensitive method using multiplex real-time PCR for diagnosis of infections by Influenza A and Influenza B viruses, Respiratory Syncytial Viruses, and Parainfluenzaviruses 1, 2, 3, and 4. J Clin Microbiol 2004; 42: 1564-9.
Uncapher C R, DeWitt C M and Colonno R J. The major and minor group receptor families contain all but one human rhinovirus serotype. Virology 1991; 180:814-7
van Elden L J R, van Loon A M, van Alphen F, et al. Frequent detection of human coronaviruses in clinical specimens from patients with respiratory tract infection by use of a novel real-time reverse-transcriptase polymerase chain reaction. J Infect Dis 2004; 189:652-7.
Wang D, Coscoy L, Zylberberg M, et al. Microarray-based detection and genotyping of viral pathogens. Proc Natl Acad Sci USA 2002; 99:15687-92
Winther B, Hayden F G and Hendley J O. Picornavirus infections in children diagnosed by RT-PCR during longitudinal surveillance with weekly sampling: Association with symptomatic illness and effect of season. J Med Virol 2006; 78:644-50
Xatzipsalti M, Kyrana S, Tsolia M, et al. Rhinovirus viremia in children with respiratory infections. Am J Respir Crit. Care Med 2005; 172:1037-40

What is claimed is:

1. An isolated nucleic acid sequence comprising consecutive nucleotides having a sequence selected from the group consisting of: any sequence of SEQ ID NOs: 1-23; a variant of any sequence of SEQ ID NOs: 1-23 and having at least about 85% identity to the sequences of SEQ ID NOs: 1-23; a sequence complementary to any sequence of SEQ ID NOs: 1-23; and a sequence complementary to a variant of any sequence of SEQ ID NOs: 1-23 and having at least about 85% identity to the sequences of SEQ ID NOs: 1-23.

2. The nucleic acid of claim 1, wherein the variant has at least about 90%, about 95.5%, about 96%, about 96.5%, about 97%, about 97.5%, about 98%, about 98.5%, about 99%, about 99.5% or about 99.9% identity to that of any one of SEQ ID NOs: 1-23.

3. The isolated nucleic acid of claim 1, wherein the nucleic acid comprises consecutive nucleotides having a sequence substantially identical to any one of SEQ ID NOs: 1-23.

4. The isolated nucleic acid of claim 1, wherein the variant has at least about 95% identity to any one of SEQ ID NOs: 1-23, as determined by analysis with a sequence comparison algorithm.

5. The isolated nucleic acid of claim 4, wherein the sequence comparison algorithm is FASTA version 3.0t78 using default parameters.

6. An isolated nucleic acid comprising at least ten consecutive nucleotides having a sequence identical to a portion of any sequence selected from the group consisting of: SEQ ID NOs: 1-23; a sequence substantially identical to any one of SEQ ID NOs: 1-23; and a sequence complementary to any one of SEQ ID NOs: 1-23.

7. The isolated nucleic acid of claim 6, wherein the isolated nucleic acid is at least about 95.5%, about 96%, about 96.5%, about 97%, about 97.5%, about 98%, about 98.5%, about 99%, about 99.5% or about 99.9% complementary to SEQ ID NOs: 1-23.

8. The oligonucleotide of claim 6, wherein the oligonucleotide consists essentially of from about 10 to about 30 nucleotides.

9. An isolated polypeptide comprising consecutive amino acids having a sequence selected from the group consisting of: SEQ ID NOs: 24-35; an amino acid sequence substantially identical to any one of SEQ ID NOs: 24-35; and an amino acid sequence having at least about 90%, about 95.5%, about 96%, about 96.5%, about 97%, about 97.5%, about 98%, about 98.5%, about 99%, about 99.5% or about 99.9% identity to that of any one of SEQ ID NOs: 24-35.

10. An isolated antibody that specifically binds to the group consisting of: a polypeptide encoded by the nucleotide sequence shown in any one of SEQ ID NO: 1-23; a polypeptide comprising consecutive amino acids having a sequence selected from the group consisting of: SEQ ID NO: 24-35; and a polypeptide having at least about 90%, about 95.5%, about 96%, about 96.5%, about 97%, about 97.5%, about 98%, about 98.5%, about 99%, about 99.5% or about 99.9% identity to that of any one of SEQ ID NO: 24-35

11. The antibody of claim 10, wherein the antibody is a polyclonal antibody.

12. The antibody of claim 10, wherein the antibody is a monoclonal antibody.

13. The antibody of claim 10, wherein the antibody is human or humanized.

14. The antibody of claim 10, wherein the antibody is a chimeric antibody.

15. The antibody of claim 10, wherein the antibody specifically binds to one or more of VP1, VP2, VP3, and VP4 polypeptide(s) encoded by the nucleotide sequence shown in SEQ ID NO: 1.

16. A method for determining the presence or absence of picornavirus HRV-NY in a biological sample, the method comprising:
a) contacting nucleic acid from a biological sample with at least one primer which is a nucleic acid of claim 6,
b) subjecting the nucleic acid and the primer to amplification conditions, and
c) determining the presence or absence of amplification product, wherein the presence of amplification product indicates the presence of RNA associated with picornavirus in the sample.

17. The method of claim 16, further comprising:
a) contacting a biological sample with an antibody that specifically binds to a VP 1, VP2, VP3 and VP4 polypeptide encoded by SEQ ID NO: 1, and
b) determining whether or not the antibody binds to an antigen in the biological sample, wherein binding indicates that the biological sample contains picronavirus HRV-NY.

18. The method of claim 17, wherein the determining comprises use of a lateral flow assay or ELISA

19. The method of claim 17, wherein the determining comprises determining whether the antibodies are IgM antibodies, wherein detection of IgM antibodies is indicative of a recent infection of the sample by a picornavirus HRV-NY.

20. The method of claim 17, wherein the antibody is the antibody of claim 10.

21. A method for reducing the levels of a viral protein, viral mRNA or viral titer in a cell in a subject comprising: administering an iRNA agent to a subject, wherein the iRNA agent comprises a sense strand having at least 15 contiguous nucleotides complementary to gene from a picornavirus comprising a nucleic acid sequence selected from the group of sequences consisting of SEQ ID NO: 1-23 and an antisense strand having at least 15 contiguous nucleotides complementary to the sense strand.

22. The method of claim 21, further comprising co-administering a second iRNA agent to the subject, wherein the second iRNA agent comprises a sense strand having at least 15 or more contiguous nucleotides complementary to second gene from the picornavirus comprising a nucleic acid sequence selected from the group of sequences consisting of SEQ ID NO: 1-23 and an antisense strand having at least 15 or more contiguous nucleotides complementary to the sense strand.


SEQUENCE LISTING (47 sequences)

SEQ ID NO: 1 - DNA, 7056 bp, Human rhinovirus
gttatagttgctcccactataaccacccacgcggtgttgtggacttgtatttcggtacacttccatgccagttttatcttcccccctgcaacacttagaagaaatgtacttatggaccaataggcggcggccacccaggtcgttgacggtcaagcacttctgtctccccggtgatactggatacgctttacccaaggcgaaaaccgggctcatcgttatccgcaaagtgcctacgagaagtctagtagcactctgaaaacctatgactggtcgctccactgcaatcccagcagtagacctggcagatggggctagacataccccaccagcgatggtggtctagcccgcgtggctgcctgcacaccctatgggtgtgaagccagaaagtggacagggtgtgaagaacctattgtgctcactttgagtcctccggcccctgaatgtggctaatcctaaccctgcaaccgttgcgcacaacccagtgcgtatgcggtcgtaatgagcaattgcgggatggaaccaactactttgggtgtccgtgtttcttgttattcttttatttgcttatggtgacactgtatagctatacattgtcatcatgggcgctcaggtctccaaacaaagcgtgggagctcatgaaactatggtgcatgctggctctggtgcagttgttaaatacttcaatatcaattattataaggatgctgctagctccggtttaaccaaacaagatttctcacaagatccatccaagtttactcaacctgtagcagatctattaacaaatccggctttgatgtccccatcagttgaggcgtgcggatactccgataggctcaagcaaatcactatcggaagctccactatcacgacacaagattctgtcaacaccatagtagcctatggtgagtggcctagttatttgtcagatttagatgcctcatctgttgataaacccacacatcctgaaacatcggctgacagattctacactttggacagtgtgcaatggggtggcagttccaaaggatggtggtggaagttgccagattgcttgaagaacatgggtatatttggacagaacatgtactaccacgctatgggaaggtctggttacatcatacacacccagtgcaatgccaccaagttccacagtggatgtctactagtagcggttgtccctgaacaccagctagcttacattggggttgatgcacaagttagctacaaacatacccacccaggtgaacgtggacacgaaataggccgcaacacaaatagagatgataaccagccagatgaaaaccctttttttaactgtaatggaacactattaggcaatttgactattttccctcaccagctcataaacctgaggacaaacaactccagcagcatagttgtaccttacatcaactgtacaccaatggatagtatgttacgccacaacaatgtctcccttgtaattatacccatctgtcctttaaaaactcccagtggagcacctcgtaccttaccaattactgtttcaattgctccagacagatctgaatcttctggtgccaggcagtccgccactcgccagggtttgccaaccagaatgcccagtggtgccaaccagttcatgacaacagaggatgagcaatcaggtaatatattacccaactttagccctacaaaacagatacacataccaggtcagataaaaacaatcatggacatggctagggtagactccttcatgcctataaacaacctacagtcacaactccaagatgtgggggtctataacattacattgactggtacatccaacaacaggatactagccatacccttggatatgagtaacacactgttttctaccacgctaatgggtgaaattctaaattacttctcaaactggtcaggttcaatcaaattaacattcctgtgtgtgtgtgatagttttagtactggaaagttcttaatagcatacaccccccctggtgctagcatcccagctaaccgcaccgacgcaatgcttggtacacacatcatatgggacttgggtttgcagtcatcatgtcatatggttatcccatggatcagttctcacttcttcagaagaacaagaaaggacaatttcacagagggtgggtttgtgacgctgtggcaccaaaccgcattcgtttccaatggtcaggtggggtctattatggtcacttgctctgcgtgttctgacatgtcagtacgcatgcttcgggacactccaatgatgaagcaagaaattaatattgcacaaaacccagtggagaagttcattgatcaaaccctagaggaggtactggtggtgccagacacacaggcatcagggccagtccataccaccaaacctcaggctttgggcgcactagagatcggcgctactgcagatgtgggaccggaggcaatgatagaaaccaggtatgttatgaacagcaacaccaacgccgaagcagcggtggaaaactttttaggtagatcagccttatgggccaacttaacactgcaaaacagttttgttgcatgggacattaactttcaagagcatgctcaagtcagaaagaaatttgaaatgttcacttatgttagatttgacttagagatcactatagtcaccaacaatgcaggcttaatgcagattatgtatgtgcccccaggagctagagccccagcaaataaggatagcaaggagtgggattcagcctcaaacccaagtgtgttttatcaacctcactctgggtttcccagattcacaatcccatttactggattgggatcggcttactacatgttctatgatggatatgatggcacagaagctggcaacatacaatatgggatatcacgaacaaatgacatgggatcactatgtatacgtgcactggatgacaccacaaagaatgatgtgaaagtgtttgctaagcccaaacacacaacagcatggattccaagaccccctagggccacccagtacactctcaaatatagcacaaactacaatgtcatcaagaaaggtacaacaagtgatcttgagcagaaacacttcttgacttataggacagacataacaaatgtgggacccagtgacatgtttgtacacactaaagaggctgtttataaatgtgctcatttaactacaccttctgagaaaactatactactagcaatttcctcagacctccagatagatagcgcagatgaacctggtcctgacatcatacctacatgtgactgcaccacaggaacatacttctgcaaaagcatggacaggtactacccagtagaattcagacaccatagctggtatgagatacaggaatcaatctattacccaaagcatatacaatatgacatcctaattggtgaaggaccatgctctccaggggattgtggtggtaaactactgtgtgtacatggaacaattggaatgatcactgctggtggagaaaaccatgtggcttttatagatctgagaaactatagctcacttagtgaacatcaaggtgttacagattacatcacccagctaggtagtgcatttggtgatggattcacaagttccataaggcaaacacttatgggtgcttgtgatgtgtctgacaaattaaccagcaaaataattaagtggactattagactaatcagtgctttatctatcatagttagaaatagtacagatactcctacaatcatagccaccttagcattgcttggttgttctggttctccatggcgatacctgaaagataaggtttgcaggtggttaggtatcgatccaccaccaagtagacagggtgattcatggctcaagaagtttacagagttctgcaatgcagcccgtggactggaatggattggagacaaactttctaaatttattgattggctcaaaggaaaaatactgcccaccctccggcgcaagtcagagacactcaaggaatgcaaaaagatcccgttgtacaaggaacaagtgagagcgtttgctacagcctcagaagatgcacaggatgaactgatggtcaacataagcaaactggagaagggccttcaagaccttgcacccctttacgcttgtgaggccaagcaggtgcgcgagatgtccagagatttacaacgcatgatgtgttacaaaaaaactcatagaacagaaccagtttgcatattgttgcacggccaacctggctgtggaaagtcactagcctccaagatagttgccagagggctgacaaatgaaagtaacgtctattcactaccaccagatccaaaatattttgatgggtacaaccaacaagaagtggtcataatggatgacgttgggcagaatcccgatggtaaagatttgagtttcttttgtcagatggtctctagtactgagtttataacaccaatggccaatttggaggacaagggtcgagcattcaccagtgactatgttatcgctagcactaacatgactgttctcaccccacccactgtttcatcacctgaagccattgacaggaggttctttcttgactgtgatgtgaagatcatgccagcatacaatacaaacaacctcttgaatgtagctaaggcactcctcccatgcactgactgccctaagccagagttctataagcaatgctgtccgctaatttgtggaaaggcgcttattctacaaaacaagaggacaaaggccagctactccataaacactgtggtgcagcaattgcgccaggaaaagaaaaacaggaagtgtgtggaacacaatcttacagccatattccagggcctaggagatgacactacaccagggtttattatagaccttctcagtgcatccaaagatccaaaagttatagaattctgtgaaaaagaaggctggatcaaacaatccaaatgttcaaaaatagaacgtgatttcaactatgcccaatactgtataaactgtgtaggtagtattgtccttattcttggaacagtatacgccctctacaaactcatgtgcattgcccaagggccttatactggattaccccaacccaaaccaagaaagccagaattaagaagagcagtggcacagggtccagaacatgaattcggaatggccctgctcaaaagaaactgccatatagctacaacagatagaggagattttaacttgcttggaatccatgacaactgtgctgtactacctacacatgcacaggcggatggcacaattttgattgatggtgttgaaacaaaaatcttaaagcaaacaattgttacagatgagagtgatgtggacacagaaataaccatcttgtggctggaccgcaatgaaaagttcagggatattaggagattcctgcccgactatcaaagagattggcagaacatgcgcctagttaccaacgtgcccaaattcccaatgcttgatatcgaggttggggatgtggtgccatatggtgatattaacctgagcggtaacccaacaactaggctgctgaaatatgactatccaaccaagccaggtcagtgcggcggtgtaatcctaaacacaggtaacataataggtattcatgtaggaggaaatggtagagttggttattgtgcatcactgacaaaaagttactttgccaccacacagggtgaaatagtcaacaaatgcaaggtgcaggatgtggggctaaagcccattaacaccccagaacattcaaagctgtatccaagtgtattcttccatgtgtttcctggagaaaaagaacctgccgccctcacccagaaagatcctagactagagacagacctcaagacagctgtcatgtctaagtacaaaggaaatgtccatattgaaatgagtgagaacattcatatagctgtggaacactattcagctcagttgtttatgctagacatcaacccagaacctataaccttggaacaagctgtgtatggcatgcagaatctagaaccattagatctcacaactagtgcaggatttccatatgttactatgggagtaaagaagagggatattctaaacagaatcacaaaggacactaccaaactgcaagagatgattgacacatatggccttgatttaccttatattacatatctaaaggatgagttgcggagtcctgctaagataaaagctggaaaaaccagggctatagaagcagctagcatgaatgatacagcaaactttagaagagtttttggcaacttgtatgccagtttccatgctaatccaggagttcttactggctctgctataggttgtgatcctgacatcttttggtcacaactatattcatctatggaaaagcatctgttagtgtttgactataccaattatgatggaagtttgcatccagtgtggtttcaagcactggaacaactactgaataaccttggttttcctggggaattagcctacaaactgtgtaatacaactcacatatacaaagatgagatgtactctgtgaaaggagggatgccatcaggtatttcaggcacttcaatctttaacacaattatcaataatattataatcagaacattagtgttggacacttacaaaggaattgaattggacaaactcaaaatagtagcatatggagatgatgtcatagcctcatatcctgaggagcttgaccctgctgagattgctatctctggattaaagtacgggttgacaataacccctgcagataagtcaagccagtttacaaaaattgactggactaatgccacttttttaaagagaggcttcaaggctgatgaaaaacattctttccttattcatccaacatttcctgaaagtgaaatctttgaatcaattagatggaccagagatccaaagaacacccaagatcatgtctattcattatgtctcctaatgtggcataatggagaaaagccttacagagagtttgtagacaggatcaggaggactgacgttggccgtaggctgtatcttccaccatacagtctgctgcagcgagcttggatcgataaatttatctaaatgctttaatgtagaaacctatctgtgtaggaatagga

SEQ ID NO: 2 - DNA, 2697 bp, Human rhinovirus
  modified_base (346)..(355): a, c, g, t, unknown or other
ttccccggcacccttatatacgcttcacccgaggcgaaaaatgaggttgtcgttatccgcaaagtgcctacgagaagcctagtaacactttgaaaacccatggttggtcgttcagctgtttacccaacagtagacctggcagatgaggctagacactccccaccagcgatggtggtctagcctgcgtggctgcctgcacaccctgccgggtgtgaagccagaaagtggacaaggtgtgaagagcctattgtgctcactttgagtcctccggcccctgaatgtggctaaccctaaccccgtagctgtttcttgtaacccggcatgtatgcagtcgtaatgggcaacnnnnnnnnnntaccnagnactttgggtgnccgtgtttcctgttttactttttcattgcttatggtgacattgtatctgatacacttgttaccatgggcgctcaagtatctagacaaagtgttggaagccatgagacaatgatccatgctgggactggggctgtggtcaaatatttcaatgttaactactacaaagatgcagcaagttctggattaaccaaacaggatttctcccaagatccatctaaattcactcaacctgtagcagatattttaacaaaccctgcattgatgtccccctcaattgaggcatgtgggttttccgataggctcaagcaaatcactatcggaaactccactattacaacacaagatgctgttaacacaattgttgcatatggtgaatggcctagttacttatcagacatagatgcaacttcagttgataaacccacccatcctgaaacatcatcagataggttctacacgctgaagagcgtcatttgggaggttggatcatcaggctggtggtggaaattaccagactgtctaagggacatgggtgtgtttggacaaaacatgtaccaccatgccatgggaagatctgggtatattatacacactcagtgcaatgctaccaaatttcatagtggttgcttactagtagcagtggtgcctgaacaccagttggcatacattggaggtgacaacacccgagtcaaatacaagcatacacatccaggtgaattgggccacaaaattggtagtaactcagaaagaggtgataaccaaccagatgaaaactctttcttcaactgcaatggaactttgctaggaaacttaaccatcttcccacatcaactcataaacttgaggacaaacaactcaagcaccattgttgtgccctatatcaactgcacacctatggacagcatgttgcgtcataataatgtgtcacttgttattatacctatatgccctctgcgagcgccagctacagctccctcaaccttgccaattacaatatctattgcccccatcaaatcagaattttcgggtgcgagacagtctgcaaaagcgcanggtctaccagtgagaatgccaagtggagccnatcagttcatgacaccngaggatgaacagtcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatactggctataccactagacatgagcaatacacttttctctacaacgcttatgggggaagttctaaactactactcaaattggtctgggtcaatcaaacttacattcttttgtgtttgtgacagcttcagtactggcaagttcttgatagcatacacccctccaggtgctgatatcccagagtcacgccgtgatgcaatgctcggcacgcacgtcgtgtgggacctgggcctccaatcgtcatgtcaccttgttgtgccctggatcagctctcacttcttcagaaggaccagaaaggataattacacagagagtgggttcgttacattgtggcaccagacagcattcgtgtccaatggaacttcaggatccataatggtcacttgctctgcatgctctgacatgtcagtccgcatgttgcgagatacgcccatgatgaaacaagaagacaacatcacccaaaatccagtggaagagtttgtagagcacacattaaaagaggtgttggtagttccagacactcaagcttcagggcctgtccataccaccaaaccacaagctctaggtgctgtagaaataggagctactgctgatgtgggcccagaaacattaattgaaacaagatacgtaatgaatgacaacacaaatgctgaagcagcagtagagaacttcctgggaagatctgctttatgggcaaacctaagactagaccagggtttcaggaaatgggagataaacttccaagaacatgccagagtttgtagagcacacattaaaagaggtgttggtagttccagacactcaagcttcagggcctgtccataccaccaaaccacaagctctaggtgctgtagaaataggagctactgctgatgtgggcccagaaacattaattgaaacaagatacgtaatgaatgacaacacaaatgctgaagcagcagtagagaacttcctgggaagatctgctttatgggcaaacctaagactagaccagggtttcaggaaatgggagataaacttccaagaacatgcc

SEQ ID NO: 3 - DNA, 201 bp, Unknown
  Description of Unknown Picornavirus sequence
atgggcgctcaagtatctaggcaaagtgttgggagccatgaaacaatgatccacgctggaacaggagctgtggtgaaatatttcaatgtcaactattacaaggatgcagcaagttctgggttaactaagcaagatttctctcaagacccttccaaattcacccaacctgtggcagacattttatcaaatccagcactaatg

SEQ ID NO: 4 - DNA, 201 bp, Unknown
  Description of Unknown Picornavirus sequence
atgggcgctcaggtctccaaacaaagcgtgggagctcatgaaactatggtgcatgctggctctggtgcagttgttaaatacttcaatatcaattattataaggatgctgctagctccggtttaaccaaacaagatttctcacaagatccatccaagtttactcaacctgtagcagatctattaacaaatccggctttgatg

SEQ ID NO: 5 - DNA, 201 bp, Unknown
  Description of Unknown Picornavirus sequence
atgggcgctcaggtctccaaacaaagcgtgggagctcatgaaactatggtgcatgctggctctggtgcagttgttaaatacttcaatatcaattattataaggatgctgctagctccggtttaaccaaacaagatttctcacaagatccatccaagtttactcaacctgtagcagatctattaacaaacccggctttgatg

SEQ ID NO: 6 - DNA, 201 bp, Unknown
  Description of Unknown Picornavirus sequence
atgggtgcacaggtcagcaaacaaaatgttggctctcatgagagtggcatttcagcatcatcaggatcggttattaagtattttaatatcaactactacaaagattcagctagcagtggattgagcaaacaagacttctccatggatcctgaaaagttcacaaaaccaatagcagaggttatgaccaatcctgctcttatg

SEQ ID NO: 7 - DNA, 201 bp, Unknown
  Description of Unknown Picornavirus sequence
atgggcgctcaagtatctagacaaagtgttggaagccatgagacaatgatccatgctgggactggggctgtggtcaaatatttcaatgttaactactacaaagatgcagcaagttctggattaaccaaacaggatttctcccaagatccatctaaattcactcaacctgtagcagatattttaacaaaccctgcattgatg

SEQ ID NO: 8 - DNA, 201 bp, Unknown
  Description of Unknown Picornavirus sequence
atgggcgcccaagtgagcaaacaaaatgttggttcgcatgagaacagtgtttctgccagtggaggttcagtcatcaagtattttaacatcaattactacaaggactcagctagttctggtctcaccaaacaggacttctcccaagacccttcaaagtttacacagcccatagctgatgtgttgactaatcctgcactcatg

SEQ ID NO: 9 - DNA, 201 bp, Unknown
  Description of Unknown Picornavirus sequence
atgggcgctcaggtctccaaacaaagtgtgggagctcatgaaactatggtgcatgctggctctggtgcagttgttaaatactttaatatcaattactataaggatgctgctagctccggtttaaccaaacaagacttctcacaagatccatccaagtttactcaacctgtagcagatctattaacaaatccggctttgatg

SEQ ID NO: 10 - DNA, 201 bp, Unknown
  Description of Unknown Picornavirus sequence
atgggcgcacaggtcagcaagcaaaatgtcggttcgcacgagaatgttgtctctgctaacaatggctcagtcattaagtatttcaacatcaattattacaaagactcagctagctcaggtttgagcaaacaagatttttcccaagatccgtccaaatttactcaaccactagttgatactttaaccaaccctgcactcatg

SEQ ID NO: 11 - DNA, 351 bp, Unknown
  Description of Unknown Picornavirus sequence
cccggctcaagaggaatatgccttgcaaagggctgaagccccgagagtcgttatccgcatacatactacgcaaagcctagtactgtcctggaagtttattggttggtcgctccgtcaataccccattgatagacctggcagatgaggcaggaaactccccactggcgacagtgttcctgcctgcgtggctgcctgcacactcttacgggtgtgaagccaataaatggacagggtgagaagagccccgtgtgctatgtatgagtcctccggcccctgaatgcggctaatccaaccccacagctgcttcacacaaagggggtgtgttgacagtcgtaatgggtaacggacgac

SEQ ID NO: 12 - DNA, 345 bp, Unknown
  Description of Unknown Picornavirus sequence
ccggtgatactggatacgctttacccaaggcgaaaaccgggctcatcgttatccgcaaagtgcctacgagaagtctagtagcactctgaaaacctatgactggtcgctccactgcaatcccagcagtagacctggcagatggggctagacataccccaccagcgatggtggtctagcccgcgtggctgcctgcacaccctatgggtgtgaagccagaaagtggacagggtgtgaagaacctattgtgctcactttgagtcctccggcccctgaatgtggctaatcctaaccctgcaaccgttgcgcacaaccgggtgtgtatgcggtcgtaatgagcaattgcgg

SEQ ID NO: 13 - DNA, 67 bp, Unknown
  Description of Unknown Picornavirus sequence
taaccctgcagccgttgcgcacaacccagtgcgtatgcggtcgtaatgagcaattgcgggatggaac

SEQ ID NO: 14 - DNA, 345 bp, Unknown
  Description of Unknown Picornavirus sequence
ttccccggcacccttatatacgcttcacccgaggcgaaaaatgaggttgtcgttatccgcaaagtgcctacgagaagcctagtaacactttgaaaacccatggttggtcgttcagctgtttacccaacagtagacctggcagatgaggctagacactccccaccagcgatggtggtctagcctgcgtggctgcctgcacaccctgccgggtgtgaagccagaaagtggacaaggtgtgaagagcctattgtgctcactttgagtcctccggcccctgaatgtggctaaccctaaccccgtagctgtttcttgtaacccggcatgtatgcagtcgtaatgggcaac

SEQ ID NO: 15 - DNA, 348 bp, Unknown
  Description of Unknown Picornavirus sequence
ggtgatactggatacgctttacccaaggcgaaaaccgggctcatcgttatccgcaaagtgcctacgagaagtctagtagcactctgaaaacctatgactggtcgctccactgcaatcccagcagtagacctggcagatggggctagacataccccaccagcgatggtggtctagcccgcgtggctgcctgcacaccctatgggtgtgaagccagaaagtggacagggtgtgaagaacctattgtgctcactttgagtcctccggcccctgaatgtggctaatcctaaccctgcagccgtttcgcacaacggagtgcgtatgcggtcgtaatgagcaattgcgggatgc

SEQ ID NO: 16 - DNA, 67 bp, Unknown
  Description of Unknown Picornavirus sequence
taaccccgtagctgttgcatgcaacccagcatgtatgcagtcgtaatgggtaactatgggatggaac

SEQ ID NO: 17 - DNA, 355 bp, Unknown
  Description of Unknown Picornavirus sequence
gggtgtgtgaatagaccctaccagggttgaagctgtagcactcgttatccgcatggctactacgcaaatgttagtaacaccctctattgtacttgggatttcgctccgcagataccccatctgtagattagggcgatgaggctacacactccccactggcgacagtggtgtagcctgcgtggcgccctacctgggtctcctgacccaggattccacttgcaagacagggtgtgaaggcacaagtgtgctagtcatgagtcctccggcccctgaatgcggctaatcttaaccccgtagccatttcagacaaaggagggtctgtggatggtcgtaatgggcaactacgggatggaac

SEQ ID NO: 18 - DNA, 66 bp, Unknown
  Description of Unknown Picornavirus sequence
aaccccacagctattgcacacaagccagtgtgtatatagtcgtaatgagcaattgtgggacggaac

SEQ ID NO: 19 - DNA, 67 bp, Unknown; identical to SEQ ID NO: 13
  Description of Unknown Picornavirus sequence

SEQ ID NO: 20 - DNA, 60 bp, Unknown
  Description of Unknown Picornavirus sequence
gcaaccgttgcgcacaacccagtgcgtatgcggtcgtaatgagcaattgcgggatggaac

SEQ ID NO: 21 - DNA, 66 bp, Unknown
  Description of Unknown Picornavirus sequence
aaccccacagctgccgcacacaaaccagtgtgttgacagtcgtaatgggtaactgtgggacggaac

SEQ ID NO: 22 - DNA, 7056 bp, Human rhinovirus; identical to SEQ ID NO: 1
  CDS (8)..(19)
  CDS (23)..(253)
  CDS (257)..(340)
  CDS (344)..(421)
  CDS (425)..(496)
  CDS (503)..(580)
  CDS (584)..(7015)
  CDS (7028)..(7045)

SEQ ID NO: 23 - DNA, 7056 bp, Human rhinovirus
tcctattcctacacagataggtttctacattaaagcatttagataaatttatcgatccaagctcgctgcagcagactgtatggtggaagatacagcctacggccaacgtcagtcctcctgatcctgtctacaaactctctgtaaggcttttctccattatgccacattaggagacataatgaatagacatgatcttgggtgttctttggatctctggtccatctaattgattcaaagatttcactttcaggaaatgttggatgaataaggaaagaatgtttttcatcagccttgaagcctctctttaaaaaagtggcattagtccagtcaatttttgtaaactggcttgacttatctgcaggggttattgtcaacccgtactttaatccagagatagcaatctcagcagggtcaagctcctcaggatatgaggctatgacatcatctccatatgctactattttgagtttgtccaattcaattcctttgtaagtgtccaacactaatgttctgattataatattattgataattgtgttaaagattgaagtgcctgaaatacctgatggcatccctcctttcacagagtacatctcatctttgtatatgtgagttgtattacacagtttgtaggctaattccccaggaaaaccaaggttattcagtagttgttccagtgcttgaaaccacactggatgcaaacttccatcataattggtatagtcaaacactaacagatgcttttccatagatgaatatagttgtgaccaaaagatgtcaggatcacaacctatagcagagccagtaagaactcctggattagcatggaaactggcatacaagttgccaaaaactcttctaaagtttgctgtatcattcatgctagctgcttctatagccctggtttttccagcttttatcttagcaggactccgcaactcatcctttagatatgtaatataaggtaaatcaaggccatatgtgtcaatcatctcttgcagtttggtagtgtcctttgtgattctgtttagaatatccctcttctttactcccatagtaacatatggaaatcctgcactagttgtgagatctaatggttctagattctgcatgccatacacagcttgttccaaggttataggttctgggttgatgtctagcataaacaactgagctgaatagtgttccacagctatatgaatgttctcactcatttcaatatggacatttcctttgtacttagacatgacagctgtcttgaggtctgtctctagtctaggatctttctgggtgagggcggcaggttctttttctccaggaaacacatggaagaatacacttggatacagctttgaatgttctggggtgttaatgggctttagccccacatcctgcaccttgcatttgttgactatttcaccctgtgtggtggcaaagtaactttttgtcagtgatgcacaataaccaactctaccatttcctcctacatgaatacctattatgttacctgtgtttaggattacaccgccgcactgacctggcttggttggatagtcatatttcagcagcctagttgttgggttaccgctcaggttaatatcaccatatggcaccacatccccaacctcgatatcaagcattgggaatttgggcacgttggtaactaggcgcatgttctgccaatctctttgatagtcgggcaggaatctcctaatatccctgaacttttcattgcggtccagccacaagatggttatttctgtgtccacatcactctcatctgtaacaattgtttgctttaagatttttgtttcaacaccatcaatcaaaattgtgccatccgcctgtgcatgtgtaggtagtacagcacagttgtcatggattccaagcaagttaaaatctcctctatctgttgtagctatatggcagtttcttttgagcagggccattccgaattcatgttctggaccctgtgccactgctcttcttaattctggctttcttggtttgggttggggtaatccagtataaggcccttgggcaatgcacatgagtttgtagagggcgtatactgttccaagaataaggacaatactacctacacagtttatacagtattgggcatagttgaaatcacgttctatttttgaacatttggattgtttgatccagccttctttttcacagaattctataacttttggatctttggatgcactgagaaggtctataataaaccctggtgtagtgtcatctcctaggccctggaatatggctgtaagattgtgttccacacacttcctgtttttcttttcctggcgcaattgctgcaccacagtgtttatggagtagctggcctttgtcctcttgttttgtagaataagcgcctttccacaaattagcggacagcattgcttatagaactctggcttagggcagtcagtgcatgggaggagtgccttagctacattcaagaggttgtttgtattgtatgctggcatgatcttcacatcacagtcaagaaagaacctcctgtcaatggcttcaggtgatgaaacagtgggtggggtgagaacagtcatgttagtgctagcgataacatagtcactggtgaatgctcgacccttgtcctccaaattggccattggtgttataaactcagtactagagaccatctgacaaaagaaactcaaatctttaccatcgggattctgcccaacgtcatccattatgaccacttcttgttggttgtacccatcaaaatattttggatctggtggtagtgaatagacgttactttcatttgtcagccctctggcaactatcttggaggctagtgactttccacagccaggttggccgtgcaacaatatgcaaactggttctgttctatgagtttttttgtaacacatcatgcgttgtaaatctctggacatctcgcgcacctgcttggcctcacaagcgtaaaggggtgcaaggtcttgaaggcccttctccagtttgcttatgttgaccatcagttcatcctgtgcatcttctgaggctgtagcaaacgctctcacttgttccttgtacaacgggatctttttgcattccttgagtgtctctgacttgcgccggagggtgggcagtatttttcctttgagccaatcaataaatttagaaagtttgtctccaatccattccagtccacgggctgcattgcagaactctgtaaacttcttgagccatgaatcaccctgtctacttggtggtggatcgatacctaaccacctgcaaaccttatctttcaggtatcgccatggagaaccagaacaaccaagcaatgctaaggtggctatgattgtaggagtatctgtactatttctaactatgatagataaagcactgattagtctaatagtccacttaattattttgctggttaatttgtcagacacatcacaagcacccataagtgtttgccttatggaacttgtgaatccatcaccaaatgcactacctagctgggtgatgtaatctgtaacaccttgatgttcactaagtgagctatagtttctcagatctataaaagccacatggttttctccaccagcagtgatcattccaattgttccatgtacacacagtagtttaccaccacaatcccctggagagcatggtccttcaccaattaggatgtcatattgtatatgctttgggtaatagattgattcctgtatctcataccagctatggtgtctgaattctactgggtagtacctgtccatgcttttgcagaagtatgttcctgtggtgcagtcacatgtaggtatgatgtcaggaccaggttcatctgcgctatctatctggaggtctgaggaaattgctagtagtatagttttctcagaaggtgtagttaaatgagcacatttataaacagcctctttagtgtgtacaaacatgtcactgggtcccacatttgttatgtctgtcctataagtcaagaagtgtttctgctcaagatcacttgttgtacctttcttgatgacattgtagtttgtgctatatttgagagtgtactgggtggccctagggggtcttggaatccatgctgttgtgtgtttgggcttagcaaacactttcacatcattctttgtggtgtcatccagtgcacgtatacatagtgatcccatgtcatttgttcgtgatatcccatattgtatgttgccagcttctgtgccatcatatccatcatagaacatgtagtaagccgatcccaatccagtaaatgggattgtgaatctgggaaacccagagtgaggttgataaaacacacttgggtttgaggctgaatcccactccttgctatccttatttgctggggctctagctcctgggggcacatacataatctgcattaagcctgcattgttggtgactatagtgatctctaagtcaaatctaacataagtgaacatttcaaatttctttctgacttgagcatgctcttgaaagttaatgtcccatgcaacaaaactgttttgcagtgttaagttggcccataaggctgatctacctaaaaagttttccaccgctgcttcggcgttggtgttgctgttcataacatacctggtttctatcattgcctccggtcccacatctgcagtagcgccgatctctagtgcgcccaaagcctgaggtttggtggtatggactggccctgatgcctgtgtgtctggcaccaccagtacctcctctagggtttgatcaatgaacttctccactgggttttgtgcaatattaatttcttgcttcatcattggagtgtcccgaagcatgcgtactgacatgtcagaacacgcagagcaagtgaccataatagaccccacctgaccattggaaacgaatgcggtttggtgccacagcgtcacaaacccaccctctgtgaaattgtcctttcttgttcttctgaagaagtgagaactgatccatgggataaccatatgacatgatgactgcaaacccaagtcccatatgatgtgtgtaccaagcattgcgtcggtgcggttagctgggatgctagcaccagggggggtgtatgctattaagaactttccagtactaaaactatcacacacacacaggaatgttaatttgattgaacctgaccagtttgagaagtaatttagaatttcacccattagcgtggtagaaaacagtgtgttactcatatccaagggtatggctagtatcctgttgttggatgtaccagtcaatgtaatgttatagacccccacatcttggagttgtgactgtaggttgtttataggcatgaaggagtctaccctagccatgtccatgattgtttttatctgacctggtatgtgtatctgttttgtagggctaaagttgggtaatatattacctgattgctcatcctctgttgtcatgaactggttggcaccactgggcattctggttggcaaaccctggcgagtggcggactgcctggcaccagaagattcagatctgtctggagcaattgaaacagtaattggtaaggtacgaggtgctccactgggagtttttaaaggacagatgggtataattacaagggagacattgttgtggcgtaacatactatccattggtgtacagttgatgtaaggtacaactatgctgctggagttgtttgtcctcaggtttatgagctggtgagggaaaatagtcaaattgcctaatagtgttccattacagttaaaaaaagggttttcatctggctggttatcatctctatttgtgttgcggcctatttcgtgtccacgttcacctgggtgggtatgtttgtagctaacttgtgcatcaaccccaatgtaagctagctggtgttcagggacaaccgctactagtagacatccactgtggaacttggtggcattgcactgggtgtgtatgatgtaaccagaccttcccatagcgtggtagtacatgttctgtccaaatatacccatgttcttcaagcaatctggcaacttccaccaccatcctttggaactgccaccccattgcacactgtccaaagtgtagaatctgtcagccgatgtttcaggatgtgtgggtttatcaacagatgaggcatctaaatctgacaaataactaggccactcaccataggctactatggtgttgacagaatcttgtgtcgtgatagtggagcttccgatagtgatttgcttgagcctatcggagtatccgcacgcctcaactgatggggacatcaaagccggatttgttaatagatctgctacaggttgagtaaacttggatggatcttgtgagaaatcttgtttggttaaaccggagctagcagcatccttataataattgatattgaagtatttaacaactgcaccagagccagcatgcaccatagtttcatgagctcccacgctttgtttggagacctgagcgcccatgatgacaatgtatagctatacagtgtcaccataagcaaataaaagaataacaagaaacacggacacccaaagtagttggttccatcccgcaattgctcattacgaccgcatacgcactgggttgtgcgcaacggttgcagggttaggattagccacattcaggggccggaggactcaaagtgagcacaataggttcttcacaccctgtccactttctggcttcacacccatagggtgtgcaggcagccacgcgggctagaccaccatcgctggtggggtatgtctagccccatctgccaggtctactgctgggattgcagtggagcgaccagtcataggttttcagagtgctactagacttctcgtaggcactttgcggataacgatgagcccggttttcgccttgggtaaagcgtatccagtatcaccggggagacagaagtgcttgaccgtcaacgacctgggtggccgccgcctattggtccataagtacatttcttctaagtgttgcaggggggaagataaaactggcatggaagtgtaccgaaatacaagtccacaacaccgcgtgggtggttatagtgggagcaactataac

SEQ ID NO: 24 - protein, 2139 aa, Human rhinovirus
MGAQVSKQSVGAHETMVHAGSGAVVKYFNINYYKDAASSGLTKQDFSQDPSKFTQPVADLLTNPALMSPSVEACGYSDRLKQITIGSSTITTQDSVNTIVAYGEWPSYLSDLDASSVDKPTHPETSADRFYTLDSVQWGGSSKGWWWKLPDCLKNMGIFGQNMYYHAMGRSGYIIHTQCNATKFHSGCLLVAVVPEHQLAYIGVDAQVSYKHTHPGERGHEIGRNTNRDDNQPDENPFFNCNGTLLGNLTIFPHQLINLRTNNSSSIVVPYINCTPMDSMLRHNNVSLVIIPICPLKTPSGAPRTLPITVSIAPDRSESSGARQSATRQGLPTRMPSGANQFMTTEDEQSGNILPNFSPTKQIHIPGQIKTIMDMARVDSFMPINNLQSQLQDVGVYNITLTGTSNNRILAIPLDMSNTLFSTTLMGEILNYFSNWSGSIKLTFLCVCDSFSTGKFLIAYTPPGASIPANRTDAMLGTHIIWDLGLQSSCHMVIPWISSHFFRRTRKDNFTEGGFVTLWHQTAFVSNGQVGSIMVTCSACSDMSVRMLRDTPMMKQEINIAQNPVEKFIDQTLEEVLVVPDTQASGPVHTTKPQALGALEIGATADVGPEAMIETRYVMNSNTNAEAAVENFLGRSALWANLTLQNSFVAWDINFQEHAQVRKKFEMFTYVRFDLEITIVTNNAGLMQIMYVPPGARAPANKDSKEWDSASNPSVFYQPHSGFPRFTIPFTGLGSAYYMFYDGYDGTEAGNIQYGISRTNDMGSLCIRALDDTTKNDVKVFAKPKHTTAWIPRPPRATQYTLKYSTNYNVIKKGTTSDLEQKHFLTYRTDITNVGPSDMFVHTKEAVYKCAHLTTPSEKTILLAISSDLQIDSADEPGPDIIPTCDCTTGTYFCKSMDRYYPVEFRHHSWYEIQESIYYPKHIQYDILIGEGPCSPGDCGGKLLCVHGTIGMITAGGENHVAFIDLRNYSSLSEHQGVTDYITQLGSAFGDGFTSSIRQTLMGACDVSDKLTSKIIKWTIRLISALSIIVRNSTDTPTIIATLALLGCSGSPWRYLKDKVCRWLGIDPPPSRQGDSWLKKFTEFCNAARGLEWIGDKLSKFIDWLKGKILPTLRRKSETLKECKKIPLYKEQVRAFATASEDAQDELMVNISKLEKGLQDLAPLYACEAKQVREMSRDLQRMMCYKKTHRTEPVCILLHGQPGCGKSLASKIVARGLTNESNVYSLPPDPKYFDGYNQQEVVIMDDVGQNPDGKDLSFFCQMVSSTEFITPMANLEDKGRAFTSDYVIASTNMTVLTPPTVSSPEAIDRRFFLDCDVKIMPAYNTNNLLNVAKALLPCTDCPKPEFYKQCCPLICGKALILQNKRTKASYSINTVVQQLRQEKKNRKCVEHNLTAIFQGLGDDTTPGFIIDLLSASKDPKVIEFCEKEGWIKQSKCSKIERDFNYAQYCINCVGSIVLILGTVYALYKLMCIAQGPYTGLPQPKPRKPELRRAVAQGPEHEFGMALLKRNCHIATTDRGDFNLLGIHDNCAVLPTHAQADGTILIDGVETKILKQTIVTDESDVDTEITILWLDRNEKFRDIRRFLPDYQRDWQNMRLVTNVPKFPMLDIEVGDVVPYGDINLSGNPTTRLLKYDYPTKPGQCGGVILNTGNIIGIHVGGNGRVGYCASLTKSYFATTQGEIVNKCKVQDVGLKPINTPEHSKLYPSVFFHVFPGEKEPAALTQKDPRLETDLKTAVMSKYKGNVHIEMSENIHIAVEHYSAQLFMLDINPEPITLEQAVYGMQNLEPLDLTTSAGFPYVTMGVKKRDILNRITKDTTKLQEMIDTYGLDLPYITYLKDELRSPAKIKAGKTRAIEAASMNDTANFRRVFGNLYASFHANPGVLTGSAIGCDPDIFWSQLYSSMEKHLLVFDYTNYDGSLHPVWFQALEQLLNNLGFPGELAYKLCNTTHIYKDEMYSVKGGMPSGISGTSIFNTIINNIIIRTLVLDTYKGIELDKLKIVAYGDDVIASYPEELDPAEIAISGLKYGLTITPADKSSQFTKIDWTNATFLKRGFKADEKHSFLIHPTFPESEIFESIRWTRDPKNTQDHVYSLCLLMWHNGEKPYREFVDRIRRTDVGRRLYLPPYSLLQRAWIDKFI

SEQ ID NO: 25 - protein, 699 aa, Human rhinovirus
  MOD_RES (330)..(330): Any amino acid
MGAQVSRQSVGSHETMIHAGTGAVVKYFNVNYYKDAASSGLTKQDFSQDPSKFTQPVADILTNPALMSPSIEACGFSDRLKQITIGNSTITTQDAVNTIVAYGEWPSYLSDIDATSVDKPTHPETSSDRFYTLKSVIWEVGSSGWWWKLPDCLRDMGVFGQNMYHHAMGRSGYIIHTQCNATKFHSGCLLVAVVPEHQLAYIGGDNTRVKYKHTHPGELGHKIGSNSERGDNQPDENSFFNCNGTLLGNLTIFPHQLINLRTNNSSTIVVPYINCTPMDSMLRHNNVSLVIIPICPLRAPATAPSTLPITISIAPIKSEFSGARQSAKAXGLPVRMPSGAXQFMTPEDEQSXXXXXXXXXXXXXXXXXXXXXXXXXXXXXXXXXXXXXXXXXXXXXXXXXXXXXXXXXXILAIPLDMSNTLFSTTLMGEVLNYYSNWSGSIKLTFFCVCDSFSTGKFLIAYTPPGADIPESRRDAMLGTHVVWDLGLQSSCHLVVPWISSHFFRRTRKDNYTESGFVTLWHQTAFVSNGTSGSIMVTCSACSDMSVRMLRDTPMMKQEDNITQNPVEEFVEHTLKEVLVVPDTQASGPVHTTKPQALGAVEIGATADVGPETLIETRYVMNDNTNAEAAVENFLGRSALWANLRLDQGFRKWEINFQEHARVCRAHIKRGVGSSRHSSFRACPYHQTTSSRCCRNRSYC

SEQ ID NO: 26 - protein, 687 aa, Human rhinovirus
  MOD_RES (266)..(266): Any amino acid
MGAQVSKQSVGAHETMVHAGSGAVVKYFNINYYKDAASSGLTKQDFSQDPSKFTQPVADLLTNPALMSPSVEACGYSDRLKQITIGSSTITTQDSVNTIVAYGEWPSYLSDLDASSVDKPTHPETSADRFYTLDSVQWGGSSKGWWWKLPDCLKNMGIFGQNMYYHAMGRSGYIIHTQCNATKFHSGCLLVAVVPEHQLAYIGVDAQVSYKHTHPGERGHEIGRNTNRDDNQPDENPFFNCNGTLLGNLTIFPHQLINLRTNNSSXIVVPYINCTPMDSMLRHNNVSLVIIPICPLKTPSGAPRTLPITVSIAPDRSESSGARQSATRQGLPTRMPSGANQXMTTEDEQSGNILPNFSPTKQIHIPGQIKTIMDMARVDSFMPINNLQSQLQDVGVYNITLTGTSNNRILAIPLDMSNTLFSTTLMGEILNYFSNWSGSIKLTFLCVCDSFSTGKFLIAYTPPGASIPANRTDAMLGTHIIWDLGLQSSCHMVIPWISSHFFRRTRKDNFTEGGFVTLWHQTAFVSNGQVGSIMVTCSACSDMSVRMLRDTPMMKQEINIAQNPVEKFIDQTLEEVLVVPDTQASGPVHTTKPQALGALEIGATADVGPEAMIETRYVMNSNTNAEAAVENFLGRSALWANLTLQNSFVAWDINFQEHAQVRKKFEMFTYVRFDLEITIVTNNAGLM

SEQ ID NO: 27 - protein, 93 aa, Human rhinovirus
EFVEHTLKEVLVVPDTQASGPVHTTKPQALGAVEIGATADVGPETLIETRYVMNDNTNAEAAVENFLGRSALWANLRLDQGFRKWEINFQEHA

SEQ ID NO: 28 - protein, 143 aa, Human rhinovirus
MGAQVSRQSVGSHETMIHAGTGAVVKYFNVNYYKDAASSGLTKQDFSQDPSKFTQPVADILSNPALMSPSIEACGFSDRLKQITIGNSTITTQDAVNTIVAYGEWPSYLSDMDATSVDKPTHPETSSDRFYTLNSVTWQGGSL

SEQ ID NO: 29 - protein, 144 aa, Human rhinovirus
MGAQVSRQSVGSHETMIHAGTGAVVKYFNVNYYKDAASSGLTKQDFSQDPSKFTQPVADILTNPALMSPSIEACGFSDRLKQITIGNSTITTQDAVNTIVAYGEWPSYLSDIDATSVDKPTHPETSSDRFYTLKSVIWEVGSSG

SEQ ID NO: 30 - protein, 143 aa, Human rhinovirus
MGAQVSKQNVGSHESGISASSGSVIKYFNINYYKDSASSGLSKQDFSMDPEKFTKPIAEVMTNPALMSPSIEACGFSDRLKQITIGSSTITTQDTLNTIVAYGEWPQYLSDLDASAIDKPTHPETSTDRFYTLASVDWTQSSK

SEQ ID NO: 31 - protein, 140 aa, Human rhinovirus
MGAQVSKQNVGSHENVVSANNGSVIKYFNINYYKDSASSGLSKQDFSQDPSKFTQPLVDTLTNPALMSPSVEACGFSDRLKQITIGNSTITTQDSINTVLAYGEWPQYLSGIDATSVDKPTHPETSADRFYTLSSIEWTT

SEQ ID NO: 32 - protein, 144 aa, Human rhinovirus
MGAQVSKQSVGAHETMVHAGSGAVVKYFNINYYKDAASSGLTKQDFSQDPSKFTQPVADLLTNPALMSPSVEACGYSDRLKQITIGSSTITTQDSVNTIVAYGEWPSYLSDLDASSVDKPTHPETSADRFYTLDSVQWGGSSKG

SEQ ID NO: 33 - protein, 143 aa, Human rhinovirus
MGAQVSKQSVGAHETMVHAGSGAVVKYFNINYYKDAASSGLTKQDFSQDPSKFTQPVADLLTNPALMSPSVEACGYSDRLKQITIGSSTITTQDSVNTIVAYGEWPSYLSDLDASSVDKPTHPETSADRFYTLDSVQWGGSSK

SEQ ID NO: 34 - protein, 143 aa, Human rhinovirus
MGAQVSKQNVGSHENSVSASGGSVIKYFNINYYKDSASSGLTKQDFSQDPSKFTQPIADVLTNPALMSPTVEACGFSDRLKQITIGNSTITTQDSLNTIVAYGEWPEYLSDLDATSVDKPTHPETSSDRFYTLESVMWHGSSR

SEQ ID NO: 35 - protein, 144 aa, Human rhinovirus; identical to SEQ ID NO: 32

SEQ ID NO: 36 - protein, 4 aa, Human rhinovirus
LLPL

SEQ ID NO: 37 - protein, 7 aa, Human rhinovirus
CGPRNIN

SEQ ID NO: 38 - protein, 6 aa, Human rhinovirus
NKIRNE

SEQ ID NO: 39 - protein, 4 aa, Human rhinovirus
QHKC

SEQ ID NO: 40 - protein, 33 aa, Human rhinovirus
SSSRELPGKICFMGKPKTRPGFQEMGDKLPRTC

SEQ ID NO: 41 - protein, 77 aa, Human rhinovirus
PPTRCCGLVFRYTSMPVLSSPLQHLEEMYLWTNRRRPPRSLTVKHFCLPGDTGYALPKAKTGLIVIRKVPTRSLVAL

SEQ ID NO: 42 - protein, 28 aa, Human rhinovirus
KPMTGRSTAIPAVDLADGARHTPPAMVV

SEQ ID NO: 43 - protein, 26 aa, Human rhinovirus
PAWLPAHPMGVKPESGQGVKNLLCSL

SEQ ID NO: 44 - protein, 24 aa, Human rhinovirus
VLRPLNVANPNPATVAHNPVRMRS

SEQ ID NO: 45 - protein, 26 aa, Human rhinovirus
AIAGWNQLLWVSVFLVILLFAYGDTV

SEQ ID NO: 46 - protein, 2144 aa, Human rhinovirus
LYIVIMGAQVSKQSVGAHETMVHAGSGAVVKYFNINYYKDAASSGLTKQDFSQDPSKFTQPVADLLTNPALMSPSVEACGYSDRLKQITIGSSTITTQDSVNTIVAYGEWPSYLSDLDASSVDKPTHPETSADRFYTLDSVQWGGSSKGWWWKLPDCLKNMGIFGQNMYYHAMGRSGYIIHTQCNATKFHSGCLLVAVVPEHQLAYIGVDAQVSYKHTHPGERGHEIGRNTNRDDNQPDENPFFNCNGTLLGNLTIFPHQLINLRTNNSSSIVVPYINCTPMDSMLRHNNVSLVIIPICPLKTPSGAPRTLPITVSIAPDRSESSGARQSATRQGLPTRMPSGANQFMTTEDEQSGNILPNFSPTKQIHIPGQIKTIMDMARVDSFMPINNLQSQLQDVGVYNITLTGTSNNRILAIPLDMSNTLFSTTLMGEILNYFSNWSGSIKLTFLCVCDSFSTGKFLIAYTPPGASIPANRTDAMLGTHIIWDLGLQSSCHMVIPWISSHFFRRTRKDNFTEGGFVTLWHQTAFVSNGQVGSIMVTCSACSDMSVRMLRDTPMMKQEINIAQNPVEKFIDQTLEEVLVVPDTQASGPVHTTKPQALGALEIGATADVGPEAMIETRYVMNSNTNAEAAVENFLGRSALWANLTLQNSFVAWDINFQEHAQVRKKFEMFTYVRFDLEITIVTNNAGLMQIMYVPPGARAPANKDSKEWDSASNPSVFYQPHSGFPRFTIPFTGLGSAYYMFYDGYDGTEAGNIQYGISRTNDMGSLCIRALDDTTKNDVKVFAKPKHTTAWIPRPPRATQYTLKYSTNYNVIKKGTTSDLEQKHFLTYRTDITNVGPSDMFVHTKEAVYKCAHLTTPSEKTILLAISSDLQIDSADEPGPDIIPTCDCTTGTYFCKSMDRYYPVEFRHHSWYEIQESIYYPKHIQYDILIGEGPCSPGDCGGKLLCVHGTIGMITAGGENHVAFIDLRNYSSLSEHQGVTDYITQLGSAFGDGFTSSIRQTLMGACDVSDKLTSKIIKWTIRLISALSIIVRNSTDTPTIIATLALLGCSGSPWRYLKDKVCRWLGIDPPPSRQGDSWLKKFTEFCNAARGLEWIGDKLSKFIDWLKGKILPTLRRKSETLKECKKIPLYKEQVRAFATASEDAQDELMVNISKLEKGLQDLAPLYACEAKQVREMSRDLQRMMCYKKTHRTEPVCILLHGQPGCGKSLASKIVARGLTNESNVYSLPPDPKYFDGYNQQEVVIMDDVGQNPDGKDLSFFCQMVSSTEFITPMANLEDKGRAFTSDYVIASTNMTVLTPPTVSSPEAIDRRFFLDCDVKIMPAYNTNNLLNVAKALLPCTDCPKPEFYKQCCPLICGKALILQNKRTKASYSINTVVQQLRQEKKNRKCVEHNLTAIFQGLGDDTTPGFIIDLLSASKDPKVIEFCEKEGWIKQSKCSKIERDFNYAQYCINCVGSIVLILGTVYALYKLMCIAQGPYTGLPQPKPRKPELRRAVAQGPEHEFGMALLKRNCHIATTDRGDFNLLGIHDNCAVLPTHAQADGTILIDGVETKILKQTIVTDESDVDTEITILWLDRNEKFRDIRRFLPDYQRDWQNMRLVTNVPKFPMLDIEVGDVVPYGDINLSGNPTTRLLKYDYPTKPGQCGGVILNTGNIIGIHVGGNGRVGYCASLTKSYFATTQGEIVNKCKVQDVGLKPINTPEHSKLYPSVFFHVFPGEKEPAALTQKDPRLETDLKTAVMSKYKGNVHIEMSENIHIAVEHYSAQLFMLDINPEPITLEQAVYGMQNLEPLDLTTSAGFPYVTMGVKKRDILNRITKDTTKLQEMIDTYGLDLPYITYLKDELRSPAKIKAGKTRAIEAASMNDTANFRRVFGNLYASFHANPGVLTGSAIGCDPDIFWSQLYSSMEKHLLVFDYTNYDGSLHPVWFQALEQLLNNLGFPGELAYKLCNTTHIYKDEMYSVKGGMPSGISGTSIFNTIINNIIIRTLVLDTYKGIELDKLKIVAYGDDVIASYPEELDPAEIAISGLKYGLTITPADKSSQFTKIDWTNATFLKRGFKADEKHSFLIHPTFPESEIFESIRWTRDPKNTQDHVYSLCLLMWHNGEKPYREFVDRIRRTDVGRRLYLPPYSLLQRAWIDKFI

SEQ ID NO: 47 - protein, 6 aa, Human rhinovirus
CRNLSV